(19) United States
(12) Patent Application Publication
(10) Pub. No.: US 2024/0279601 A1
(43) Pub. Date: Aug. 22, 2024

(54) METHODS OF GENERATING SACRAL NEURAL CREST LINEAGES AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Lorenz STUDER, New York, NY (US); Yujie Fan, New York, NY (US)

(73) Assignees: SLOAN-KETTERING INSTITUTE FOR CANCER RESEARCH, New York, NY (US); MEMORIAL SLOAN-KETTERING CANCER CENTER, Nnew York, NY (US); MEMORIAL HOSPITAL FOR CANCER AND ALLIED DISEASES, New York, NY (US)

(21) Appl. No.: 18/542,134

(22) Filed: Dec. 15, 2023

Related U.S. Application Data
(63) Continuation of application No. PCT/US22/34148, filed on Jun. 20, 2022.
(60) Provisional application No. 63/212,210, filed on Jun. 18, 2021.

(51) Int. Cl. C12N 5/0793; A61K 35/30

(52) U.S. Cl. CPC C12N 5/0619; A61K 35/30; C12N 2501/115; C12N 2501/15; C12N 2501/155; C12N 2501/16; C12N 2501/19; C12N 2501/415; C12N 2506/45

(57) ABSTRACT

The present disclosure relates to methods for generating sacral neural crest lineage cells and enteric neurons. Also provided are sacral neural crest lineage cells and enteric neurons generated by the presently disclosed methods and compositions comprising such cells. The present disclosure further provides uses of the sacral neural crest lineage cells and enteric neurons for preventing, modeling, and/or treating of enteric nervous system disorders.

### CROSS-REFERENCE TO RELATED APPLICATIONS

This application is a continuation of International Patent application no. PCT/US2022/034148, filed Jun. 20, 2022, which claims priority to U.S. Provisional Application No. 63/212,210, filed Jun. 18, 2021, the content of which are incorporated by reference in their entireties, and to which priority is claimed.

### GRANT INFORMATION

The present disclosure was made with government support under Grant No. NS099270-05 awarded by the National Institute of Health. The government has certain rights in the disclosure.

### SEQUENCE LISTINGS

A Sequence Listing conforming to the rules of WIPO Standard ST.26 is hereby incorporated by reference. Said Sequence Listing has been filed as an electronic document via PatentCenter in ASCII format encoded as XML. The electronic document, created on Dec. 15, 2023, is entitled “072734_1536_SL.xml”, and is 8,391 bytes in size.

### 1. TECHNICAL FIELD

The present disclosure relates to methods for generating sacral neural crest lineage cells, sacral neural crest lineage cells generated by such methods and compositions comprising such cells. The present disclosure also provides uses of the sacral neural crest lineage cells and compositions comprising thereof for preventing, modeling, and/or treating of enteric nervous system disorders.

### 2. BACKGROUND

The enteric nervous system (ENS) is the largest and most diverse component of the human autonomic nervous system. The ENS is derived from the vagal neural crest cells (VNCs) and sacral neural crest cells (SNCs) and represents a complex network of neurons with dozens of distinct neurotransmitter subtypes essential for gastro-intestinal (GI) function. Defects in ENS development are responsible for disorders including Hirschsprung disease (HD), gastroparesis, irritable bowel syndrome, hypertrophic pyloric stenosis, esophageal atresia, and Chagas's disease. Defects in the ENS is also linked to various neurological disorders ranging from Parkinson disease to Alzheimer's disease.

Despite the importance of the ENS in human disease, its development remains poorly understood owing to the lack of an easily accessible model system. Moreover, compared to the VNCs, very little is known about the SNCs, such as their lineage potentials, functionality and overall contribution to ENS-related diseases. Therefore, there remains a need for an in vitro method and protocol of generating SNCs directly from human stem cells.

### 3. SUMMARY OF THE INVENTION

The present disclosure relates to methods for generating sacral neural crest lineage cells, sacral neural crest lineage cells generated by such methods, compositions comprising such cells, and uses of such cells and compositions for preventing, modeling, and/or treating of enteric nervous system disorders (e.g., Hirschsprung disease (HD)). The present disclosure is partly based on the discovery that activation of FGF and Wnt signaling promote in vitro patterning of caudal Hox codes in cells, and GDF11 promotes the transition from trunk neural crest cells to sacral neural crest lineage cells.

In certain embodiments, the present disclosure provides in vitro methods for inducing differentiation of stem cells, comprising inducing activation of wingless (Wnt) signaling, activation of fibroblast growth factor (FGF) signaling, and sacral neural crest patterning in the stem cells to obtain a population of differentiated cells expressing at least one marker indicating a sacral neural crest lineage.

In certain embodiments, the methods comprise contacting the stem cells with at least one activator of Wnt signaling, at least one activator of FGF signaling, and at least one molecule that induces sacral neural crest patterning.

In certain embodiments, the cells are contacted with the at least one molecule that induces sacral neural crest patterning for at least about 1 day, and/or the sacral neural crest patterning is induced for at least about 1 day. In certain embodiments, the cells are contacted with the at least one molecule that induces sacral neural crest patterning for up to about 20 days, and/or the sacral neural crest patterning is induced for up to about 20 days. In certain embodiments, the cells are contacted with the at least one molecule that induces sacral neural crest patterning for about 3 days, and/or the sacral neural crest patterning is induced for about 3 days (e.g., 3 days or 4 days).

In certain embodiments, the cells are contacted with the at least one activator of FGF signaling for at least about 1 day, and/or the activation of FGF signaling is induced for at least about 1 day. In certain embodiments, the cells are contacted with the at least one activator of FGF signaling for up to about 8 days, and/or the activation of FGF signaling is induced for at least about 8 days. In certain embodiments, the cells are contacted with the at least one activator of FGF signaling for about 3 days, and/or the activation of FGF signaling is induced for about 5 days (e.g., 3 days, 4 days, or 5 days).

In certain embodiments, the cells are contacted with the at least one activator of Wnt signaling for at least about 6 days, and/or the activation of Wnt signaling is induced for at least about 6 days. In certain embodiments, the cells are contacted with the at least one activator of Wnt signaling for up to about 25 days, and/or the activation of Wnt signaling is induced for up to about 20 days. In certain embodiments, the cells are contacted with at least one activator of Wnt signaling for about 20 days, and/or the activation of Wnt signaling is induced for about 20 days (e.g., 20 days or 21 days).

In certain embodiments, the contact of the cells with the at least one molecule that induces sacral neural crest patterning is not initiated on the same day as the initial contact of the cell with the at least one activator of Wnt signaling, and/or the induction of sacral neural crest patterning is not initiated on the same day as the activation of Wnt signaling. In certain embodiments, the contact of the cells with the at least one molecule that induces sacral neural crest patterning is initiated after the initial contact of the cell with the at least one activator of Wnt signaling, and/or the induction of sacral neural crest patterning is initiated after the initial induction of the activation of Wnt signaling.

In certain embodiments, the contact of the cells with the at least one molecule that induces sacral neural crest patterning is initiated on the same day as the initial contact of the cell with the at least one activator of Wnt signaling, and/or the induction of sacral neural crest patterning is initiated on the same day as the activation of Wnt signaling.

In certain embodiments, the contact of the cells with the at least one activator of FGF signaling is initiated on the same day as the initial contact of the cell with the at least one activator of Wnt signaling, and/or the induction of activation of FGF signaling is initiated on the same day as the activation of Wnt signaling.

In certain embodiments, the at least one molecule that induces sacral neural crest patterning is a member of transforming growth factor β (TGFβ) family. In certain embodiments, the at least one molecule that induces sacral neural crest patterning comprises a Bone morphogenetic protein (BMP). In certain embodiments, the at least one molecule that induces sacral neural crest patterning comprises a growth differentiation factor (GDF). In certain embodiments, the at least one molecule that induces sacral neural crest patterning is selected from the group consisting of Growth differentiation factor 11 (GDF11), GDF8, and combinations thereof.

In certain embodiments, the at least one activator of FGF signaling is a member of FGF1 subfamily, FGF4 subfamily, or FGF8 subfamily. In certain embodiments, the at least one activator of FGF signaling is selected from the group consisting of FGF, FGF2, FGF4, FGF6, FGF7, FGF8, FGF17, FGF18, and combination thereof.

In certain embodiments, the at least one activator of Wnt signaling activates canonical Wnt signaling. In certain embodiments, the at least one activator of Wnt signaling comprises an inhibitor of glycogen synthase kinase 3β (GSK3β) signaling. In certain embodiments, the at least one activator of Wnt signaling is selected from the group consisting of CHIR99021, CHIR98014, AMBMP hydrochloride, LP 922056, Lithium, BIO, SB-216763, Wnt3A, Wnt1, Wnt5a, derivatives thereof, and combinations thereof. In certain embodiments, the at least one activator of Wnt signaling comprises CHIR99021.

In certain embodiments, the cells are further contacted with at least one inhibitor of Small Mothers Against Decapentaplegic (SMAD) signaling, and/or the method further comprises inducing inhibition of SMAD signaling.

In certain embodiments, the cells are contacted with the at least one inhibitor of SMAD signaling for at least about 1 day, and/or the inhibition of SMAD signaling is induced for at least about 1 day. In certain embodiments, the cells are contacted with the at least one inhibitor of SMAD signaling for up to about 20 days, and/or the inhibition of SMAD signaling is induced for up to about 20 days. In certain embodiments, the cells are contacted with the at least one inhibitor of SMAD signaling for about 15 days, and/or the inhibition of SMAD signaling is induced for about 15 days. In certain embodiments, the cells are contacted with the at least one inhibitor of SMAD signaling for 16 days, 17 days, or 18 days, and/or the inhibition of SMAD signaling is induced for 16 days, 17 days, or 18 days.

In certain embodiments, the contact of the cells with the at least one molecule that induces sacral neural crest patterning is not initiated on the same day as the initial contact of the cell with the at least one inhibitor of SMAD signaling. In certain embodiments, the contact of the cells with the at least one molecule that induces sacral neural crest patterning is initiated after the initial contact of the cells with the at least one inhibitor of SMAD signaling.

In certain embodiments, the at least one inhibitor of SMAD signaling comprises an inhibitor of TGFβ/Activin-Nodal signaling, and/or the inhibition of SMAD signaling comprises inhibition of TGFβ/Activin-Nodal signaling. In certain embodiments, the at least one inhibitor SMAD signaling further comprises an inhibitor of bone morphogenetic protein (BMP) signaling, and/or the inhibition of SMAD signaling further comprises inhibition of BMP signaling. In certain embodiments, the at least one inhibitor of TGFβ/Activin-Nodal signaling comprises an inhibitor of ALK5. In certain embodiments, the at least one inhibitor of TGFβ/Activin-Nodal signaling is selected from the group consisting of SB431542, derivatives of SB431542, and combinations thereof.

In certain embodiments, the derivative of SB431542 comprises A83-01, and/or RepSox. In certain embodiments, the at least one inhibitor of TGFβ/Activin-Nodal signaling comprises SB431542.

In certain embodiments, the at least one inhibitor of BMP signaling is selected from the group consisting of LDN193189, Noggin, dorsomorphin, derivatives of LDN193189, derivatives of Noggin, derivatives of dorsomorphin, and combinations thereof. In certain embodiments, the at least one inhibitor of BMP comprises LDN-193189.

In certain embodiments, the cells are contacted with at least one bone morphogenetic protein (BMP), and/or the method further comprises inducing activation of BMP signaling. In certain embodiments, the cells are contacted with the at least one BMP for at least about 1 day, and/or the activation of BMP signaling is induced for at least about 1 day. In certain embodiments, the cells are contacted with the at least one BMP for up to about 25 days, and/or the activation of BMP signaling is induced for up to about 25 days. In certain embodiments, the cells are contacted with at least one BMP for about 20 days, and/or the activation of BMP signaling is induced for about 20 days (e.g., 20 days or 21 days).

In certain embodiments, the at least one BMP is selected from the group consisting of BMP1, BMP2, BMP3, BMP4, BMP5, BMP6, BMP7, BMP8a, BMP8b, BMP10, BMP11, BMP15, and combinations thereof. In certain embodiments, the at least one BMP comprises BMP2, BMP4, or a combination thereof.

In certain embodiments, at least about 70% of the cells express the at least one sacral neural crest lineage marker at least about 20 days from the initial contact of the stem cells with the at least one activator of Wnt signaling, and/or from the initiation of the induction of activation of Wnt signaling.

In certain embodiments, the at least one sacral neural crest lineage marker is selected from the group consisting of Hox10, HOX11, HOX12, and HOX13, combinations thereof. In certain embodiments, the differentiated cells further express at least one SOX10⁺ neural crest lineage marker. In certain embodiments, the at least one SOX10⁺ neural crest lineage marker comprises CD49D.

In certain embodiments, the stem cells are pluripotent stem cells. In certain embodiments, the stem cells are human stem cells. In certain embodiments, the stem cells are selected from the group consisting of embryonic stem cells, induced pluripotent stem cells, parthenogenetic stem cells, primordial germ cell-like pluripotent stem cells, epiblast stem cells, and F-class pluripotent stem cells, enhanced pluripotent stem cells, naive stage pluripotent stem cells, and combinations thereof.

In certain embodiments, the methods disclosed herein further comprise subject the differentiated cells to conditions favoring maturation of sacral neural crest lineage cells to cells that express at least enteric neuron marker.

In certain embodiments, the conditions favoring maturation of sacral neural crest lineage cells to cells that express at least enteric neuron marker comprise contacting the differentiated cells with at least one growth factor, at least one Wnt activator, or a combination thereof. In certain embodiments, the at least one growth factor comprises at least one FGF activator, glial cell line derived neurotrophic factor (GDNF), ascorbic acid, or a combination thereof. In certain embodiments, the differentiated cells are contacted with the at least one Wnt activator and the at least one FGF activator. In certain embodiments, the differentiated cells are contacted with the at least one Wnt activator and the at least one FGF activator for about 4 days. In certain embodiments, the differentiated cells are contacted with the at least one Wnt activator, the at least one FGF activator, GDNF, and ascorbic acid.

In certain embodiments, the at least one FGF activator is selected from the group consisting of FGF2, FGF4, FGF7, and FGF8.

In certain embodiments, the at least one Wnt activator is selected from the group consisting of CHIR99021, CHIR98014, AMBMP hydrochloride, LP 922056, Lithium, BIO, SB-216763, Wnt3A, Wnt1, Wnt5a, derivatives thereof, and combinations thereof.

In certain embodiments, the at least one enteric neuron marker is selected from the group consisting of Tuj1, MAP2, PHOX2A, PHOX2B, TRKC, ASCL1, HAND2, EDNRB, 5HT, GABA, NOS, SST, TH, CHAT, DBH, Substance P, VIP, NPY, GnRH, CGRP, and combinations thereof.

In certain embodiments, the methods disclosed herein further comprise subject the differentiated cells to conditions favoring maturation of sacral neural crest lineage cells to cells that express at least enteric glia marker.

In certain embodiments, the conditions favoring maturation of sacral neural crest lineage cells to cells expressing at least at least enteric glia marker comprise contacting the differentiated cells with at least one growth factor, at least one Wnt activator, or a combination thereof. In certain embodiments, the at least one growth factor comprises at least one FGF activator, glial cell line derived neurotrophic factor (GDNF), ascorbic acid, or a combination thereof. In certain embodiments, the differentiated cells are contacted with the at least one Wnt activator and the at least one FGF activator. In certain embodiments, the differentiated cells are contacted with the at least one Wnt activator and the at least one FGF activator for about 4 days. In certain embodiments, the differentiated cells are contacted with the at least one Wnt activator, the at least one FGF activator. GDNF, and ascorbic acid.

In certain embodiments, the at least one enteric glia marker is selected from the group consisting of GFAP, S100b, vimentin, conexin-43, SOX10, and combinations thereof.

In certain embodiments, the present disclosure provides a cell population of in vitro differentiated cells expressing at least one sacral neural crest lineage marker obtained by a presently disclosed method.

In certain embodiments, the present disclosure provides a cell population of in vitro differentiated cells expressing at least one enteric neuron marker obtained by a presently disclosed method.

In certain embodiments, the present disclosure provides a composition comprising a presently disclosed cell population. In certain embodiments, the composition is a pharmaceutical composition further comprising a pharmaceutically acceptable carrier.

In certain embodiments, the present disclosure provides a kit for inducing differentiation of stem cells, comprising: (a) at least one activator of Wnt signaling; (b) at least one activator of FGF signaling; (c) at least one molecule that induces sacral neural crest patterning; and (d) instructions for inducing differentiation of the stem cells into cells expressing at least one sacral neural crest lineage marker. In certain embodiments, the kit further comprises at least one inhibitor of SMAD signaling. In certain embodiments, the kit further comprises at least one BMP. In certain embodiments, the at least one molecule that induces sacral neural crest patterning is selected from the group consisting of GDF11, GDF8, and combinations thereof.

In certain embodiments, the present disclosure provides a kit for inducing differentiation of stem cells, comprising: (a) at least one activator of Wnt signaling; (b) at least one activator of FGF signaling; (c) at least one molecule that induces sacral neural crest patterning; (d) at least one growth factor; (e) at least one Wnt activator; and (f) instructions for inducing differentiation of the stem cells into cells expressing at least one enteric neuron marker. In certain embodiments, the kit further comprises at least one inhibitor of SMAD signaling. In certain embodiments, the kit further comprises at least one BMP. In certain embodiments, the at least one growth factor comprises FGF activators, glial cell line derived neurotrophic factor (GDNF), ascorbic acid, or a combination thereof.

In certain embodiments, the present disclosure provides a method of preventing and/or treating an enteric nervous system disorder in a subject in need thereof, comprising administering to the subject an effective amount of one of the followings: (a) the presently disclosed cell population; or (b) the presently disclosed composition. In certain embodiments, the enteric nervous system disorder is Hirschsprung's disease.

In certain embodiments, the presently disclosed cell population or the presently disclosed composition is for use in preventing and/or treating an enteric nervous system disorder in a subject in need thereof. In certain embodiments, the enteric nervous system disorder is Hirschsprung's disease.

### 4. BRIEF DESCRIPTION OF THE DRAWINGS

FIG. 1 is schematic showing of an exemplary protocol of the presently disclosed methods for differentiating stem cells into SNCs.

FIGS. 2A-2D show fully defined (E8/E6-based) neural crest differentiation strategies. FIG. 2A shows cranial neural crest cells (CNCs) and vagal neural crest cells (VNCs) differentiation protocols. FIG. 2B shows a differentiation protocol for testing the effects of FGF2 and CHIR99021 on Hox code patterning. FIG. 2C shows that activation of FGF and Wnt signaling highly induced the expression of posterior genes. FIG. 2D shows that activation of FGF and Wnt signaling induced upregulation of CDX genes but not of neural crest genes.

FIGS. 3A-3F show the role of GDF11 tor the transition from trunk to tail. FIG. 3A shows that GDF11 promoted the expression of Hox genes and the transition from trunk neural crest cells (TNCs) to sacral neural crest lineage cells (SNCs). FIG. 3B shows the characterization of the sacral neural crest at day 20 by immunoblotting. FIG. 3C shows the characterization of the sacral neural crest at day 20 by immunofluorescence. FIG. 3D shows the characterization of the sacral neural crest at day 20 by flow cytometry. FIG. 3E shows the efficacy of the differentiation method with three different iPSC cell lines. FIG. 3F shows the cumulative analysis of the data shown in FIG. 2E. FIG. 3G shows that percentage of cells that are CD49D⁺ sacral neural crest at day 14 of differentiation.

FIG. 4 shows exemplary differentiation strategies of different cells.

FIGS. 5A and 5B show that neuromesodermal progenitors (NMPs) are progenitors for posterior NC cells. FIG. 5A shows imagining characterizing NMPs. FIG. 5B shows the development of new reporter lines for NMPs.

FIG. 6 shows immunofluorescence and flow cytometry analysis of differentiated NMP reporter lines. The reporter stem cells were differentiated using an exemplary protocol of the presently disclosed methods showing in FIG. 3. Day 3 cells were collected and were analyzed using immunofluorescence and flow cytometry.

FIG. 7 shows that sorted NMP cells generated posterior NC in vitro.

FIG. 8 is a schematic showing that NMP is a progenitor for posterior NC cells.

FIG. 9 shows a heat-map of Hox genes expression during differentiation.

FIG. 10 shows that early treatment of GDF11 induced posterior Hox gene expression at a later time point.

FIG. 11 is a schematic showing the experimental design to determine whether GDF11 regulates chromatin modifications.

FIG. 12 shows ATAC-seq and RNA-seq data for detecting the working mechanism of GDF11 using Principal Component Analysis.

FIGS. 13A-13D show ATAC-seq and RNA-seq data for detecting the working mechanism of GDF11. FIGS. 13A and 138 show the ATAC-seq results. FIG. 13C shows heat-maps of the RNA-seq results indicating that early treatment of GDF11 induced posterior Hox genes expression at a later time point. FIG. 13D shows the heat-map analysis indicating that sorted pure NMP can generate posterior NC in vitro.

FIGS. 14A and 14B show the gene set enrichment in GDF11 treatment at day 3 of the differentiation. FIG. 14A shows gene sets enriched during the differentiation. FIG. 14B shows representative ATAC-seq and RNA-seq data indicating very few transcripts having different expression levels at day 7 of sacral neural crest lineage cell differentiation.

FIGS. 15A and 15B show the gene set enrichment in GDF11 treatment at day 14 of the differentiation. FIG. 15A shows gene sets enriched during the differentiation. FIG. 15B shows representative transcripts.

FIGS. 16A-16D show the different migration and invasion abilities of SNCs and VNCs. FIG. 16A shows the experimental design. FIG. 16B shows quantification of neural crest cells invasion assay and migration assay on poly-ornithine/laminin/fibronectin (PO/LM/FN)-coated dishes. FIG. 16C shows live imaging of co-cultured SNCs and VNCs in 2D. FIG. 16D shows images of co-cultured SNCs and VNCs in matrigel embedded 3D.

FIGS. 17A-17C show the differentiation of SNCs into different subtypes of enteric neurons. FIG. 17A shows an exemplary protocol of the presently disclosed methods of differentiating sacral neural crest lineage cells into enteric neurons. FIG. 17B shows representative images of cells collected at day 7 and day 20 of differentiation. FIG. 17C shows representative immunofluorescent images of cells were collected at day 60 of differentiation. Cells were stained for detection of GABA, TH, NOS, CHAT, SST, and 5′HT.

FIGS. 18A and 18B show the electrophysiological activity of enteric neurons using multi-electrode array (MEA) system. FIG. 18A shows the activity heat map and the spikes of enteric neurons which had stronger electrophysiological activity with maturation. FIG. 18B shows the spike raster activity in enteric neurons and the quantification of firing rate and bursting electrodes.

FIGS. 19A-19D show in vivo mouse experiments to compare VNCs and SNCs. VNCs and SNCs were transplanted into mouse colon. FIG. 19A shows representative images for detection of the transplanted tissues and an anatomical representation of the injection points. FIG. 19B shows the colon of a mouse model before and after injection. FIG. 19C shows that VNCs and SNCs had distinct migratory behavior after transplantation. FIG. 19D shows the whole mount staining of a tissue transplanted with GFP-labeled SNCs six months after injection.

FIGS. 20A-20C show rescue experiments with a mouse Hirschsprung's disease model. FIG. 20A is a schematic showing the rescue experiments. FIGS. 20B and 20C show survival curves (FIG. 20B) and body weight measurements (FIG. 20C).

FIG. 21 is schematic showing of an exemplary protocol of the presently disclosed methods for differentiating stem cells into SNCs.

FIGS. 22A-22G illustrate derivation of sacral NC from hPSCs. FIG. 22A shows schematic drawing of the FGF and CHIR titration experimental design. Different combinations of FGF2 and CHIR, with concentrations ranging from low to high, were added from D0-D2 on top of the established cranial NC differentiation protocol. Cells were collected and analyzed at different time points for analysis. FIG. 22B shows qRT-PCR of neural crest genes at D20, indicating the formation of neural crest cells under all conditions except for the LSB control, which induces hPSC into early neurectoderm. N=3 biological replicates. FIG. 22C shows qRT-PCR of HOX genes that indicate regional identity corresponding to distinct axial levels. HOXB4 indicates the vagal level; HOXC9 indicates the trunk level; HOXD13 indicates the sacral level. N=4 biological replicates. FIG. 22D shows qRT-PCR of HOX genes that represent distinct axial levels under the condition with or without GD F11, indicating GD F11 promotes sacral level HOX gene expression. N=3 biological replicates. Significance represents differences between GD F11 versus cultures treated without GD F11. FIG. 22E shows immunohistochemistry of sacral NC at D20, co-staining of SOX10 and posterior HOX proteins HOXC9 and HOXD13 shows most cells become sacral NC. Scale bars, 50 pm. FIG. 22F shows flow cytometry of sacral NC for CD49D⁺ and p75NTR at D20. Left panel depicts one representative replicate. Right panel is the quantitative data of CD49D⁺ positive cell percentage. N=7 biological replicates. FIG. 22G shows schematic summary of protocols that generate NC cells at different axial levels. Data are present as Mean±SEM; ns: not significant P>0.05; *P≤0.05; **P≤0.01; ***P≤0.001; ****P≤0.0001.

FIGS. 23A-23G illustrated GDF1-mediated expression of 5′ HOX genes via modulation of RA signaling. FIG. 23A shows qRT-PCR analysis showing expression of HOX genes representing distinct axial levels at different time points of sacral NC differentiation and showing the progressive expression of HOX genes from 3′ HOX genes to 5′ HOX genes. HOXB4 (vagal level) expression occurs first, followed by HOXC9 (trunk level) and HOXD13 (sacral level). N=4 biological replicates. FIG. 23B shows schematic drawing of RNA seq and ATAC seq experimental design comparing conditions with or without GD F11. FIG. 23C shoes PCA plot of the RNA sequencing data (without hESC samples). FIG. 23D shows PCA plot of the ATAC sequencing data (without hESC samples). FIG. 23E shows heat map of RNA-seq expression (row-based z-score of variance-stabilized counts), showing increased expression of HOXA genes over time and greater expression of more posterior HOXA genes under GD F11 treatment. FIG. 23F shows qRT-PCR showing expression of GRHL factors: GRHL1, GRHL2, GRHL3 from RNA seq. N=3 biological replicates. FIG. 23G shows chromatin opening status of GRHL3 locus from ATAC seq, indicating suppressed GRHL3 expression in GD F11 condition. N=3 biological replicates. FIG. 23H shows qRT-PCR showing expression of RA binding protein gene CRABP2, indicating reduced RA signaling pathway activity in the GD F11 condition at D3 and D7. N=3 biological replicates. FIG. 23I shows schematic drawing of proposed mechanism by which GD F11 promotes the generation of sacral NC by RA inhibition. FIG. 23J shows qRT-PCR showing expression of stem cell marker SOX2. N=3 biological replicates. FIG. 23K shows experimental design to test the RA hypothesis. FIGS. 23L-23O show qRT-PCR data showing expression of various genes related to RA signaling and AP identity for the experiment depicted in FIG. 23K. FIG. 23L shows expression of CRABP2, confirming the effect of RA and RA inhibitor AGN. N=3 biological replicates. FIG. 23M shows expression of anterior HOX gene HOXB2 was promoted by RA treatment. N=3 biological replicates. FIG. 23N shows expression of trunk level posterior HOX gene HOXC9 was promoted by RA inhibition treatment. N=3 biological replicates. FIG. 23O shows expression of sacral level posterior HOX gene HOXC10 was promoted by RA inhibition treatment. N=3 biological replicates. RNA sequencing data presented as counts normalized using the Median of Ratios method (DESeq2). Data are present as Mean±SEM; ns: not significant P>0.05; *P≤0.05; **P≤0.01; ***P≤0.001; ****P≤0.0001.

FIGS. 24A-24J illustrate sacral NC cells are derived from an NMP-like posterior precursor population. FIG. 24A shows co-expression of SOX2, T and CDX2 at D3 cells of sacral NC differentiation, showing most cells are triple-positive for 3 genes. Scale bars, 50 pm. FIG. 24B shows gene expression of SOX2, T and CDX2 at D3 compared with hESC (qRT-PCR). N=3 biological replicates. FIG. 24C shows flow cytometry data of D3 cells, show that around 71% cells are positive for SOX2 and T and among the double positive population, almost all cells are CDX2 positive. FIG. 24D shows Venn diagram of differentially expressed genes (t log 2(FC)t>1) representing D3 cells of our trunk differentiation protocol (labelled as BMP D3), D3 cells of our sacral differentiation (labelled as GD F D3) and D3 NMP cells from Frith et al., study (labelled as NMP-Trunk D3) (Frith et al., Elife 7.10.7554/eLife.35786, 2018). 1549 common genes were identified. FIG. 24E shows GO analysis of our GD F D3 cells, which share high similarity with BMP D3 cells and gene expression data published by Frith et al. (2018), indicating common features between these data sets. FIG. 24F shows top 25 most up and down regulated genes from the common genes in the Venn diagram presented in FIG. 24D. FIG. 24 (shows experimental design depicting use of SOX2::TdTomato and T::G FP dual reporter hESC line to test if a pure NMP-like population can give rise to sacral NC. FIG. 24H shows flow cytometry data of D3 cells using dual reporter line or H9 WT control (left). Double positive cells (constituting around 60% of the total population) are sorted out and replated for sacral NC differentiation. The purity of the sorted NMP populations is confirmed with immunostaining for SOX2 and T (right). Scale bars, 50 pm. FIG. 24I shows flow cytometry data of D20 cells from unsorted and sorted NMPs (left). Around 93% of the cells are sacral NC cells, which is comparable with the unsorted population. The sacral NC identity is confirmed with immunostaining of SOX10 and HOXD13 (right). Scale bars, 50 pm. FIG. 24J shows schematic summary of anterior and posterior NC domains originating from different precursors. Data are present as Mean±SEM; ns: not significant P>0.05; *P≤0.05; **P≤0.01; *P≤0.001; **P≤0.0001

FIGS. 25A-25L illustrate sacral NC can be directed to diverse enteric and non-enteric NC fates. FIG. 25A shows schematic of the differentiation protocols used to specify sacral NC cells towards enteric neurons (upper panel), sympathetic neurons (middle panel) and melanocytes (bottom panel). FIG. 25B shows immunostaining of SOX10, HOXD13 and TUJ1 at an early stage of enteric neuron differentiation (D30). The cells are HOXD13 positive, indicating sacral identity. Most cells have lost SOX10 expression and started expression of TUBB3 (as indicated with TUJ1 staining), indicating transition to neuronal fate from NC. Scale bars, 200 pm (left panel) and 50 pm (right panel). FIG. 25C shows immunostaining of TUJ1 on D40 of the enteric neuron differentiation. Scale bars, 200 pm (left panel) and 50 pm (right panel). FIG. 25D shows immunostaining of mature enteric neurons at D80. Staining with TUJ1 shows neurons have a more complex morphology than those at D40. Staining with neuron subtype makers, NOS, GABA, TH, CHAT, SST and 5′HT show that sacral NC-derived enteric neurons exhibit a range of different subtypes. Scale bars, 200 pm (left top panel) and 50 pm (the rest). FIGS. 25E-25O show gene expression of NC marker SOX10, enteric NC precursor marker EDNRB and neuronal marker SOX2 during enteric neuron differentiation. N=3 biological replicates. FIG. 25H shows neuron subtype composition within the culture at D80. N=2 biological replicates. FIG. 25I shows immunostaining of mature sympathetic neurons at D80. Staining with TUJ1, showing the neurons form bundle-like structures composed of long neurites distinct from the cytoarchitecture of D80 enteric neurons. Staining with TH and DBH shows the sympathetic identity of the neurons. Scale bars, 100 pm (1st and 3rd panel) and 50 pm (the rest). FIG. 25J shows gene expression (qRT-PCR) of sympathetic neuronal markers: TH, DBH, PRPH, PHOX2B at D30 and D60. N=4 biological replicates. FIG. 25K shows live imaging of sacral NC-derived melanocytes at D60 using a SOX10::G FP hESC reporter line under florescent microscope (left 3 panels, same image) and under bright field microscope (right panel, different image), showing the continuous expression of SOX10 in melanocytes, as well as presence of pigmentation. Scale bars, 100 pm. FIG. 25L shows gene expression of melanocyte markers: SOX10, hMITF, c-KIT and pigment-related genes TYPL1 PMEL at D30 and D60. N=3 biological replicates. Data are present as Mean±SEM; ns: not significant P>0.05; *P≤0.05; **P≤0.01; ***P≤0.001; ****P≤0.0001

FIG. 26A-26K illustrates vagal NC and sacral NC exhibit distinct behavior both in vitro and in vivo. FIG. 26A shows schematic drawing of experimental design used to generate the data presented in this figure. An RFP-tagged hPSC line was used for vagal NC and enteric neuron differentiation; a GFP-tagged hPSC line was used for sacral NC and enteric neuron differentiation. FIG. 26B shows invasion assay of vagal NC and sacral NC. The cells that crossed the membrane are visualized (left panel) and quantified using plater reader (right panel). Scale bars, 50 pm. N=3 biological replicates. FIG. 26C shows migration assay of vagal NC and sacral NC on PO/LM/FN 2D surface. Fluorescence image of cells that migrated out of spherical aggregate (left panel) and quantification of migration distance (as assessed using Image J; right panel). Scale bars, 500 pm. N=3 biological replicates. FIG. 26D shows 3D Matrigel embedded migration assay of co-cultured vagal NC and sacral NC. Fluorescence image of cells at 24 hours (upper panel) and at 96 hours (lower panel), show self-sorting and mutually repellent activity of vagal NC and sacral NC. Scale bars, 500 pm. FIG. 26E shows schematic drawing of experimental design in mice transplantation experiments. NC cells are cultured under non-adherent conditions to form small spheres composed of either vagal, sacral, or combined vagal/sacral NC and injected into the mouse cecum. FIG. 26F shows fluorescent images of mouse gut that were transplanted with different axial types of NC cells: VNC (left panel), SNC (middle panel), VNC+SNC (right panel). The images are taken at sequential time points after transplantation: 1 Hour (upper panel), 2 weeks (middle panel), 4 weeks (bottom panel). FIG. 26G shows co-cultured VNC (red) and SNC (green) cells undergoing ENS differentiation with replating at D10. Scale bars, 100 pm. FIG. 26H shows representative traces of electrical activity in NC-derived neurons as recorded by MEA system in over a period of 1 second. Neurons derived from VNC (upper panel) and SNC (lower panel) are shown. FIG. 26I shows spike rastergram showing 1 m of activity in neurons derived from VNC (left panel) and SNC (right panel). FIG. 26J shows mean firing rate of enteric neurons derived from VNC and SNC. FIG. 26K shows number of bursting electrodes in cultures of enteric neurons derived from VNC and from SNC.

FIGS. 27A-27I illustrate development of a cell-based therapy for HSCR disease using Ednrb KO mouse model. FIG. 27A shows staining of TUJ1 in the distal colon of WT mice and Ednrb KO mice. Scale bars, 50 pm. FIG. 27B shows comparison of life spans of Ednrb KO mice in different genetic backgrounds. Ednrb KO mice with a NSG background exhibit a sharp cut-off in life span (around 30 days) when compared to those with a B6; 129 background. N=10 different mice. FIGS. 27C and 27D show gut wall thickness in the distal colon of 4-week-old WT mice and HSCR (Ednrb KO) mice without any treatment. Scale bars, 100 pm. FIG. 27E shows survival curve of NSG/WT mice, NSG1Ednrb KO mice and KO mice following the various transplantation paradigms. N=12 for KO grafted with VNC+SNC and N=7 mice for all other groups. FIGS. 27F and 27G show gut wall thickness of 9-month-old WT mice and HSCR (Ednrb KO) mice that received VNC+SNC transplantation. Scale bars, 100 pm. FIG. 27H shows body weight of WT mice and KO mice with VNC+SNC transplantation at different time points after transplantation. N=3 different mice for 2 weeks and 7 weeks. Note: only 1 KO mouse survived until 36 weeks. FIG. 27I shows immunostaining of 9-month-old KO mouse that received VNC+SNC transplantation. R FP for VNC, indicated by white solid arrows and G FP for SNC, indicated by open arrows. Small intestine (upper panel); Cecum (middle panel), Distal colon (bottom panel). Scale bars, 100 pm. Data are present as Mean±SEM; ns: not significant P>0.05; *P≤0.05; **P≤0.01; **P≤0.001; ***P≤0.0001

FIGS. 28A-28J illustrate derivation of sacral NC from hPSCs. FIG. 28A shows qRT-PCR of CDX genes, showing a similar trend as HOX genes in response to FG F2/CHIR titration. N=3 biological replicates. FIG. 28B shows co-expression of trunk level HOX gene (HOXC9) and sacral level HOX gene (HOXD3). Scale bars, 50 pm. FIG. 28C shows qRT-PCR of posterior BOX genes relative to hESCs, confirming the sacral identity of the cells. N=7 biological replicates. FIG. 28D shows immunohistochemistry of replated monolayer of sacral NC at D20, co-staining of SOX10 with HOXC9, HOXD13 and Ki67. This demonstrates that the NC cells adopt a sacral level identity and are highly proliferative. Scale bars, 50 pm. FIGS. 28E-28G show quantification of co-localization of SOX10 with HOXC9, HOXD13 and Ki67. N=6 independent wells. FIG. 28H shows qRT-PCR showing SOX10, HOXC9 and HOXD13 expression in sacral NC derived from 3 different iPS lines. N=3 biological replicates. FIG. 28I shows flow cytometry of sacral NC for CD49D⁺ from 3 different iPSC lines. FIG. 28J shows expression of SOX10 at D7, D14 and D21 in conditions treated with different concentrations of FGF2 and CHIR. SOX10 expression is delayed in high FG F and CHIR concentration. N=3 biological replicates. Data are present as Mean t SEM; ns: not significant P>0.05; *P≤0.05; **P≤0.01; ***P≤0.001; ****P≤0.0001

FIGS. 29A-29K illustrate GDF11-mediated expression of 5′ Hox genes via modulation of RA signaling. FIG. 29A shows heat map of HOXB, HOXC, and HOXD gene expression from RNA seq in all samples at all time points. These data confirm the gradual expression of HOXgenes with time and more posterior HOXgene under GDF11 treatment conditions. FIG. 29B shows PCA plot of RNA sequencing data (with hESC samples). FIG. 29C shows PCA plot of ATAC sequencing data (with hESC samples). FIGS. 29D and 29E shows expression of RA target gene C YP26A1 and RA binding protein RBP4, indicating a less active RA signaling pathway in GD F11 condition. N=3 biological replicates. FIG. 29F shows expression of stem cell-related factor MYC (qRT-PCR). N=3 biological replicates. FIG. 29G shows expression of trunk level HOX gene HOXB6 was promoted as a result of RA inhibition in GDF11 conditions (qRT-PCR). N=3 biological replicates. FIGS. 29H and 29I show expression of sacral level posterior HOX genes HOXC11 and HOXC13 were promoted as a result of RA inhibition (qRT-PCR). N=3 biological replicates. FIG. 29J shows GSA analysis of enriched gene with GD F11 treatment at D3. Red text marks pathways-related to signaling. Blue text marks pathways related to chromatin modification. FIG. 29K shows expression of H3K27 and H3K4 related methylation and demethylation enzymes at D3 and D14 (RNAseq). N=3 biological replicates. Data are present as Mean±SEM; ns: not significant P>0.05; *P≤0.05; **P≤0.01; ***P≤0.001; ****P≤0.0001.

FIGS. 30A-30H illustrate sacral NC cells are derived from an NMP-like posterior precursor population. FIG. 30A shows evidence of co-expression of CDX2 with SOX2 and Tat D3, showing the presence the NMP-like cells at D3 (upper panel); co-expression of CDX2 with SNAIL2 and SOX/0 at D14, indicating that most cells have become NC precursors (middle panel); co-expression of CDX2 with SNAIL2 and SOX/0 at D20, indicating that most cells have become migratory NC cells (lower panel). This indicates that the NMP-like cells can give rise to NC cells. Scale bars, 50 pm. FIG. 30B shows gating strategy for SOX2, T and CDX2 flow cytometry experiments. FIG. 30C shows a scheme depicting the generation of H9 SOX2::tdTomato/T::G FP dual reporter line. FIG. 30D shows PCR analysis to verify the SOX2::tdTomato knock-in single-cell clones. FIG. 30E shows PCR analysis to verify T::G FP knock-in single-cell clones. FIG. 30F shows analysis by fluorescence microscope analysis of H9 SOX2::tdTomato/T::G FP dual reporter line #6 at the hESC stage and at the hESC-derived mesendoderm stage. Scale bars, 250 pm. FIG. 30G shows karyotyping results of H9 SOX2::tdTomato/T::G FP dual reporter line #6. FIG. 30H shows gating strategy for SOX2, T dual reporter line sorting experiments.

FIGS. 31A-31E illustrate sacral NC can give rise to different subtypes of enteric neurons. FIG. 31A shows immunostaining of TH and GABA in mature enteric neurons at D80. Stitched images of whole well (left panel) and magnified (right panel) images show the uneven distribution of the various neuronal subtypes. Scale bars, 500 pm (left panel) and 50 pm (right panel). FIG. 31B shows immunostaining of TUJ1 in mature sympathetic neurons at D80. Stitched images of whole well (left panel) and magnified (right panel) images to show the unique cytoarchitecture of the neurons. Scale bars, 500 pm (left panel) and 50 pm (right panel). FIG. 31C shows immunostaining of ISL1 in mature sympathetic neurons at D80. Scale bars, 50 pm. FIG. 31D shows live imaging of sacral NC derived melanocytes at D30 with SOX/0::G FP reporter line under florescent microscope (Left 3 panels) and bright field microscope (right panel), showing the continuous expression of SOX/0 in melanocytes, and lack of pigment production during early stages of melanocyte differentiation. Scale bars, 100 pm. FIG. 31E shows cell pellets of melanocytes showing pigmentation at D30 and D37.

FIGS. 32A-32G illustrate Vagal NC and Sacral NC exhibit distinct behavior both in vitro and in vivo. FIG. 32A shows scratch assay of co-cultured vagal NC and sacral NC on PO/LM/FN. Fluorescence image of cells and their migration at different time points: 0 hour (upper panel), 24 hours (middle panel), 48 hours (bottom panel). Scale bars, 50 pm. FIG. 32B shows 3D Matrigel embedded migration assay of co-cultured NC with different combination of vagal NC and sacral NC. Co-culture of VNC derived from G FP line and VNC derived from R FP line (upper panel); Co-culture of SNC derived from G FP line and SNC derived from R FP line (middle panel); Co-culture of VNC derived from G FP line and SNC derived from R FP line (bottom panel); Taken together with FIG. 5D, this indicates that the differences in migratory path are caused by different NC identity and not by differential properties of the reporter lines. Scale bars, 500 pm. FIG. 32C shows qRT-PCR of SOX10 and HOX genes to confirm the vagal and sacral NC identity. N=6 biological replicates. FIGS. 32D and 32E show surface area VNC or SNC cells in response to different growth factors. 1 equates to standard concentration (100 nM and 25 ng/ml for EDN3 and GDN F respectively), 0.1 equates to 1/10 of standard concentration and 10 equates to 10 times higher than the standard concentration. Surface area is quantified by ImageJ. N=4 biological replicates. FIG. 32F shows fluorescent images of mouse gut that were transplanted with different axial level of NC cells with split channels: VNC (upper panel), SNC (middle panel), VNC+SNC (bottom panel). The images are taken at different time points after transplantation: 1 Hour (left panel), 2 weeks (middle panel), 4 weeks (right panel). FIG. 32G shows heat map and representative neuron electrical activity of SNC derived neurons (left 2 panels) and VNC derived neurons (right 2 panels) at D40 and D90, indicating the increase of neuron maturation with time. Data are present as Mean±SEM; ns: not significant P>0.05; *P≤0.05; **P≤0.01; ***P≤0.001; ****P≤0.0001

FIGS. 33A-33H illustrate development of a cell-based therapy for HSCR disease using Ednrb KO mouse model. FIG. 33A shows NSGIEdnrb KO mice at D28 show characteristic megacolon phenotype. FIG. 33B shows H&E staining of distal colon of WT mice and Ednrb KO mice. Enteric ganglia are indicated by green open arrow. Scale bars, 100 pm. FIG. 33C shows gut wall thickness of 4-week-old WT mice and HSCR (Ednrb KO) mice without any treatment. Scale bars, 100 pm. FIG. 33D shows H&E staining of WT mice and Ednrb KO mice from proximal intestine to distal colon, showing the aganglionosis phenotype in the NSG background extended to the distal section of the small intestine. Enteric ganglia are indicated by green open arrow. Scale bars, 100 pm. FIG. 33E shows morphology of NC spheres right before transplantation. Scale bars, 50 pm. FIG. 33F shows gut wall thickness in 9-month-old WT mice and in HSCR (Ednrb KO) mice that received VNC+SNC transplantation. Scale bars, 100 pm. FIG. 33G shows whole mount staining of distal colon 6 months post sacral NC (G FP) transplantation. Co-localization of TUJ1 and G FP indicates sacral NC differentiates into neuronal cells post-transplantation. Scale bars, 50 pm. FIG. 33G shows whole mount staining of distal colon 6 months post sacral NC (G FP) transplanted. The absence of co-localization of TUJ1 with G FP positive cells indicates that in this example the sacral NC has differentiated into non-neuronal cells exhibiting glial morphologies. Scale bars, 50 pm. Data are present as Mean±SEM; ns: not significant P>0.05; *P≤0.05; **P≤0.01; ***P≤0.001; ****P≤0.0001.

### 5. DETAILED DESCRIPTION

The present disclosure relates to methods for generating sacral neural crest lineage cells, sacral neural crest lineage cells generated by such methods, compositions comprising such cells, and uses of such cells and compositions for preventing, modeling, and/or treating of enteric nervous system disorders (e.g., Hirschsprung disease (HD)).

Enteric nervous system is derived from vagal and sacral neural crests during development. The inventors previously discovered methods of differentiation of stem cells (e.g., hPSCs and iPSCs) to vagal neural crest lineage cells and further differentiation and maturation of the vagal neural crest lineage cells into enteric neurons. See WO2017112901, which is herein incorporated by reference).

The present disclosure is based, in part, on the discovery that activation of fibroblast growth factor (FGF) signaling and wingless (Wnt) signaling promote in vitro patterning of caudal Hox codes in stem cells, thereby generating trunk neural crest cells. The present disclosure further outlines that GDF11 promotes the transition from trunk to tail for neural crest cells. Contacting cells with GDF11 unexpectedly promotes the formation of sacral neural crest lineage cells in vitro. Moreover, the sacral neural crest lineage cells generated by the presently disclosed methods have high neuronal activity in vitro, and can rescue and significantly extend the lifespan of mice having HD in vivo.

Non-limiting embodiments of the present disclosure are described by the present specification and Examples.

For purposes of clarity of disclosure and not by way of limitation, the detailed description is divided into the following subsections:
5.1. Definitions;
5.2. Methods of Differentiating Stem Cells;
5.3. Cell Populations and Compositions;
5.4. Methods of Preventing and/or Treating Enteric Nervous System Disorders; and
5.6. Kits

### 5. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude. e.g., within 5-fold, or within 2-fold, of a value.

As used herein, the term “signaling” in reference to a “signal transduction protein” refers to a protein that is activated or otherwise affected by ligand binding to a membrane receptor protein or some other stimulus. Examples of signal transduction protein include, but are not limited to, a SMAD, a Wingless (Wnt) complex protein, including transforming growth factor beta (TGFβ), Activin, Nodal, glycogen synthase kinase 3β (GSK3β) proteins, bone morphogenetic proteins (BMP), and fibroblast growth factors (FGF). For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., transforming growth factor-beta (TGFβ), Activin, Nodal, bone morphogenic proteins (BMPs), etc.

“inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway (e.g., Wnt signaling pathway, and SMAD signaling). An inhibitor can be any compound or molecule that changes any activity of a named protein (signaling molecule, any molecule involved with the named signaling molecule, a named associated molecule, such as a glycogen synthase kinase 3β (GSK3β)). (e.g., including, but not limited to, the signaling molecules described herein). For example, an inhibitor of SMAD signaling can function, for example, via directly contacting SMAD, contacting SMAD mRNA, causing conformational changes of SMAD, decreasing SMAD protein levels, or interfering with SMAD interactions with signaling partners, and affecting the expression of SMAD target genes.

Inhibitors also include molecules that indirectly regulate biological activity, for example, SMAD biological activity, by intercepting upstream signaling molecules (e.g., within the extracellular domain, examples of a signaling molecule and an effect include: Noggin which sequesters bone morphogenic proteins, inhibiting activation of ALK receptors 1,2,3, and 6, thus preventing downstream SMAD activation. Likewise, Chordin, Cerberus, Follistatin, similarly sequester extracellular activators of SMAD signaling. Bambi, a transmembrane protein, also acts as a pseudo-receptor to sequester extracellular TGFβ signaling molecules). Antibodies that block upstream or downstream proteins are contemplated for use to neutralize extracellular activators of protein signaling, and the like. Although the foregoing example relates to SMAD signaling inhibition, similar or analogous mechanisms can be used to inhibit other signaling molecules. Examples of inhibitors include, but are not limited to: LDN193189 (LDN) and SB431542 (SB) (LSB) for SMAD signaling inhibition. Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activators,” as used herein, refer to compounds that increase, induce, stimulate, activate, facilitate, or enhance activation the signaling function of the molecule or pathway, e.g., Wnt signaling, FGF signaling etc.

As used herein, the term “Wnt” or “wingless” in reference to a ligand refers to a group of secreted proteins (e.g., integration 1 in humans) that are capable of interacting with a Wnt receptor, such as a receptor in the Frizzled and LRPDerailed/RYK receptor family. As used herein, the term “a Wnt or wingless signaling pathway” refers to a signaling pathway composed of Wnt family ligands and Wnt family receptors, such as Frizzled and LRPDerailed/RYK receptors, mediated with or without β-catenin. The Wnt signaling pathway include canonical Wnt signaling (e.g., mediation by β-catenin) and non-canonical Wnt signaling (mediation without β-catenin). In certain embodiments, the Wnt signaling pathway is the canonical Wnt signaling pathway.

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of midbrain DA precursors, or a population of undifferentiated stem cells, e.g., a population of A9 subtype midbrain dopamine neurons. Alternatively, the population may comprise more than one cell type, for example a mixed cell population, e.g., a cell population mixed of A9 subtype midbrain dopamine neurons and A10 subtype midbrain dopamine neurons.

As used herein, the term “stem cell” refers to a cell with the ability to divide for indefinite periods in culture and to give rise to specialized cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stein cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “pluripotent” refers to an ability to develop into the three developmental germ layers of the organism including endoderm, mesoderm, and ectoderm.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example. Takahashi and Yamanaka Cell 126, 663-676 (2006), herein incorporated by reference) into a somatic cell.

As used herein, the term “neuron” refers to a nerve cell, the principal functional units of the nervous system. A neuron consists of a cell body and its processes—an axon and at least one dendrite. Neurons transmit information to other neurons or cells by releasing neurotransmitters at synapses.

As used herein, the term “differentiation” refers to a process whereby an unspecialized embryonic cell acquires the features of a specialized cell such as a neuron, heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as midbrain dopamine neurons or precursors thereof. In references to a stem cell, “directed differentiation” refers to the use of small molecules, growth factor proteins, and other growth conditions to promote the transition of a stem cell from the pluripotent state into a more mature or specialized cell fate.

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein marker of sacral neural crest lineage. e.g., HOX10 (including HoxA10, HoxB10, HoxC10, and HoxD10), Hox11 (including HoxA11, HoxB11, HoxC11, and HoxD11), Hox12 (including HoxA12, HoxB12, HoxC12, and HoxD12), and Hox13 (including HoxA13, HoxB13, HoxC13, and HoxD13)).

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment, in vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) an ultimate parent cell in a cell line, tissue (such as a dissociated embryo, or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

### 5.2. Methods of Differentiating Stem Cells

The present disclosure provides methods for inducing in vitro differentiation of stem cells (e.g., human stem cells). In certain embodiments, the stem cells are pluripotent stem cells. In certain embodiments, the pluripotent stem cells are selected from embryonic stem cells (ESCs), induced pluripotent stem cells (iPSCs), and combinations thereof. In certain embodiments, the stem cells are multipotent stem cells. Non-limiting examples of stem cells that can be used with the presently disclosed methods include nonembryonic stem cells, embryonic stem cells, induced nonembryonic pluripotent cells, and engineered pluripotent cells. In certain embodiments, the stem cells are human stem cells. Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, enhanced pluripotent stem cells, naive stage pluripotent stem cells, somatic stem cells, cancer stem cells, or any other cell capable of lineage specific differentiation. In certain embodiments, the stem cell is a human embryonic stem cell (hESC). In certain embodiments, the stem cell is a human induced pluripotent stem cell (hiPSC). In certain embodiments, the stem cells are non-human stem cells. In certain embodiments, the stem cell is a nonhuman primate stem cell. In certain embodiments, the stem cell is a rodent stem cell.

### 5.2.1. In Vitro Differentiation of Stem Cell to Sacral Neural Crest Lineage Cells

The present disclosure provides methods for inducing in vitro differentiation of stem cells to cells expressing at least one marker indicating a sacral neural crest lineage. In certain embodiments, the methods comprise inducing activation of wingless (Wnt) signaling, activation of fibroblast growth factor (FGF) signaling, and sacral neural crest patterning in the stem cells. In certain embodiments, the methods comprise contacting the stem cells with at least one activator of Wnt signaling (also referred to as “Wnt activator”)(e.g., to induce activation of Wnt signaling), at least one activator of FGF signaling (also referred to as “FGF activators”) (e.g., to induce activation of FGF signaling), and at least one molecule that induces sacral neural crest patterning (e.g., to induce sacral neural crest patterning). In certain embodiments, the methods further comprise inducing inhibition of Small Mothers Against Decapentaplegic (SMAD) signaling. In certain embodiments, the methods further comprise contacting the stem cells with at least one inhibitor of SMAD signaling (also referred to as “SMAD inhibitors”) (e.g., to induce inhibition of SMAD signaling). In certain embodiments, the methods further comprise contacting the stem cells with at least one bone morphogenetic protein (BMP).

In certain embodiments, the differentiated cells express the at least one marker indicating a sacral neural crest lineage at least about 10 days (e.g., about 10 days, about 15 days, about 20 days, about 25 days, about 30 days, about 40 days, or about 50 days) from the initial contact of the cells with the at least one SMAD inhibitor. In certain embodiments, the differentiated cells express the at least one marker indicating a sacral neural crest lineage about 20 days (e.g., 19 days, 20 days, or 21 days) from the initial contact of the cells with the at least one activator of Wnt signaling. In certain embodiments, at least about 35% of the cells express the at least one sacral neural crest lineage marker at least about 15 days from the initial contact of the stein cells with the at least one activator of Wnt signaling, and/or from the initiation of the induction of activation of Wnt signaling. In certain embodiments, at least about 35% of the cells express the at least one sacral neural crest lineage marker at least 14 days from the initial contact of the stem cells with the at least one activator of Wnt signaling, and/or from the initiation of the induction of activation of Wnt signaling. In certain embodiments, at least about 70% of the cells express the at least one sacral neural crest lineage marker at least about 20 days from the initial contact of the stem cells with the at least one activator of Wnt signaling, and/or from the initiation of the induction of activation of Wnt signaling.

In certain embodiments, the methods for inducing in vitro differentiation of stem cells to cells expressing at least one marker indicating a sacral neural crest lineage comprise differentiation of stem cells to neuromesodermal progenitors (NMPs), and differentiation from NMPs to cells expressing at least one marker indicating a sacral neural crest lineage. In certain embodiments, the methods for in vitro differentiation of stem cells to NMPs comprise activation of Wnt signaling, activation of FGF signaling, and sacral neural crest patterning in the stem cells. In certain embodiments, the methods for in vitro differentiation of stem cells to NMPs comprise contacting the stem cells with at least one activator of Wnt signaling, at least one activator of FGF signaling, and at least one molecule that induces sacral neural crest patterning. In certain embodiments, the methods for in vitro differentiation of stem cells to NMPs further comprise inducing activation of BMP signaling in the cells.

In certain embodiments, the methods for in vitro differentiation of stem cells to NMPs further comprise inducing inhibition of SMAD signaling. In certain embodiments, the methods for in vitro differentiation of stem cells to NMPs further comprise contacting the stem cells with at least one inhibitor of SMAD signaling. In certain embodiments, the methods for in vitro differentiation of stem cells to NMPs further comprise contacting the stem cells with at least one BMP.

In certain embodiments, the methods for in vitro differentiation of stem cells to NMPs comprise contacting the stem cells with the at least one activator of Wnt signaling and the at least one activator of FGF signaling, and/or the activation of Wnt signaling and the activation of FGF signaling are induced for about 3 days, for 3 days, for 4 days, or from day 0 to day 3, in certain embodiments, the methods for in vitro differentiation of stem cells to NMPs comprise contacting the stem cells with the at least one molecule that induces sacral neural crest patterning, and/or the sacral neural crest patterning is induced for about 2 days, for 2 days, 3 days, or for 4 days, or from day 1 to day 3, or from day 0 to day 3.

In certain embodiments, the cells are not contacted with the at least one inhibitor of SMAD signaling and the at least one molecule that induces sacral neural crest patterning simultaneously, and/or the induction of the inhibition of SMAD signaling and the induction of the sacral neural crest patterning do not occur simultaneously. In certain embodiments, the cells are contacted with the at least one molecule that induces sacral neural crest patterning after their contact with the at least one inhibitor of SMAD signaling, and/or the induction of the sacral neural crest patterning takes places after the induction of the inhibition of SMAD signaling.

In certain embodiments, the methods comprise contacting the stem cells with the at least one inhibitor of SMAD signaling, the at least one activator of Wnt signaling, the at least one BMP, and the at least one activator of FGF signaling, and/or the inhibition of SMAD signaling, the activation of Wnt signaling, the activation of FGF signaling, and the activation of BMP signaling are induced, for about 3 days, for 3 days, for 4 days, or from day 0 to day 3, or from day 1 to day 3. In certain embodiments, the methods comprise contacting the cells with the at least one molecule that induces sacral neural crest patterning, and/or the sacral neural crest patterning is induced for about 2 days, for 2 days, for 3 days, or for 4 days, or from day 1 to day 3, or from day 0 day to 3.

In certain embodiments, the methods of in vitro differentiation from NMPs to cells expressing at least one marker indicating a sacral neural crest lineage comprise activation of Wnt signaling in the NMPs. In certain embodiments, the methods of in vitro differentiation from NMPs to cells expressing at least one marker indicating a sacral neural crest lineage comprise contacting the NMPs with at least one activator of Wnt signaling.

In certain embodiments, the methods of in vitro differentiation from NMPs to cells expressing at least one marker indicating a sacral neural crest lineage further comprise inducing inhibition of SMAD signaling. In certain embodiments, the methods of in vitro differentiation from NMPs to cells expressing at least one marker indicating a sacral neural crest lineage further comprise contacting the stem cells with at least one inhibitor of SMAD signaling. In certain embodiments, the methods of in vitro differentiation from NMPs to cells expressing at least one marker indicating a sacral neural crest lineage further comprise inducing activation of BMP signaling in the cells. In certain embodiments, the methods of in vitro differentiation from NMPs to cells expressing at least one marker indicating a sacral neural crest lineage further comprise contacting the NMPs with at least one BMP.

In certain embodiments, the methods of in vitro differentiation from NMPs to cells expressing at least one marker indicating a sacral neural crest lineage comprise contacting the NMPs with at least one activator of Wnt signaling, and/or the activation of Wnt signaling is induced, for about 15 days, for 15 days, 16 days, or 17 days. In certain embodiments, the methods comprise contacting the NMPs with at least one inhibitor of SMAD signaling, the at least one BMP, and at least one activator of Wnt signaling, and/or the inhibition of SMAD signaling, activation of Wnt signaling, and the activation of BMP signaling are induced, for about 15 days, for 15 days, 16 days, or 17 days.

### 5.2.1.1. Wnt Activation and Wnt Activators

In certain embodiments, the methods disclosed herein comprise inducing activation of Wnt signaling in the cells. In certain embodiments, the methods comprise inducing activation of canonical Wnt signaling in the cells. In certain embodiments, the cells are contacted with at least one Wnt activator to induce activation of Wnt signaling.

In certain embodiments, the at least one Wnt activator lowers GSK3β for activation of Wnt signaling. Thus, in certain embodiments, the Wnt activator is a GSK3β inhibitor. A GSK3β inhibitor is capable of activating a WNT signaling pathway, see e.g., Cadigan et al., J Cell Sci. 2006; 119:395-402; Kikuchi et al., Cell Signaling. 2007; 19:659-671, which are incorporated by reference herein in their entireties. As used herein, the term “glycogen synthase kinase 3β inhibitor” or “GSK3β inhibitor” refers to a compound that inhibits a glycogen synthase kinase 3β enzyme, for example, see Doble et al., J Cell Sci. 2003; 116:1175-1186, which is incorporated by reference herein in its entirety. Non-limiting examples of GSK3β inhibitors include CHIR99021, BIO ((3E)-6-bromo-3-[3-(hydroxyamino)indol-2-ylidene]-1H-indol-2-one), AMBMP hydrochloride, LP 922056, SB-216763, CHIR98014, Lithium, 3F8, and those disclosed in WO2011/149762, WO13/067362, Chambers et al., Nat Biotechnol. 2012 Jul. 1; 30(7):715-20, Kriks et al., Nature. 2011 Nov. 6; 480(7378):547-51, and Calder et al., J Neurosci. 2015 Aug. 19; 35(33):11462-81, all of which are incorporated by reference in their entireties.

Non-limiting examples of Wnt activators include CHIR99021, Wnt3A, Wnt1, Wnt5a, BIO ((3E)-6-bromo-3-[3-(hydroxyamino)indol-2-ylidene]-1H-indol-2-one), AMBMP hydrochloride, LP 922056, SB-216763, CHIR98014, Lithium, 3F8, and those disclosed in WO2011/149762, WO13/067362, Chambers et al., Nat Biotechnol. 2012 Jul. 1; 30(7):715-20, Kriks ei al., Nature, 2011 Nov. 6:480(7378):547-51, and Calder et al., J Neurosci. 2015 Aug. 19; 35(33):11462-81, all of which are incorporated by reference in their entireties. In certain embodiments, the at least one Wnt activator is a small molecule selected from CHIR99021, Wnt3A, Wnt1, Wnt5a, BIO, CHIR98014, Lithium, 3F8, derivatives thereof, and mixtures thereof. In certain embodiments, the at least one Wnt activator comprises CHIR99021 or a derivative thereof. In certain embodiments, the at least one Wnt activator comprises CHIR99021. “CHIR99021” (also known as “aminopyrimidine” or “3-[3-(2-Carboxyethyl)-4-methylpyrrol-2-methylindenyl]-2-indolinone”) refers to IUPAC name 6-(2-(4-(2,4-dichlorophenyl)-5-(4-methyl-1H-imidazol-2-yl)pyrimidin-2-ylamino)ethylamino)nicotinonitrile with the following formula.

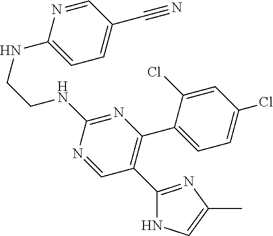 [SMILES: Cc1c[nH]c(-c2cnc(NCCNc3ccc(C#N)cn3)nc2-c2ccc(Cl)cc2Cl)n1]

CHIR99021 is highly selective, showing nearly thousand-fold selectivity against a panel of related and unrelated kinases, with an IC50=6.7 nM against human GSK3p and nanomolar IC50 values against rodent GSK3βhomologs.

In certain embodiments, the stem cells are exposed to or contacted with the at least one Wnt activator, and/or the activation of Wnt signaling is induced, for at least about 5 days. In certain embodiments, the stem cells are contacted with or exposed to the at least one Wnt activator, and/or the activation of Wnt signaling is induced, for up to about 25 days. In certain embodiments, the stem cells are contacted with or exposed to the at least one Wnt activator, and/or the activation of Wnt signaling is induced, for between about 15 days and about 25 days. In certain embodiments, the stem cells are contacted with or exposed to the at least one Wnt activator, and/or the activation of Wnt signaling is induced, for about 15 days, or about 20 days. In certain embodiments, the stem cells are contacted with or exposed to the at least one Wnt activator, and/or the activation of Wnt signaling is induced, for 15 days, 16 days, 17 days, 18 days, 19 days, 20 days, or 21 days. In certain embodiments, the stem cells are contacted with or exposed to the at least one Wnt activator, and/or the activation of Wnt signaling is induced, for about 20 days. In certain embodiments, the stem cells are contacted with or exposed to the at least one Wnt activator, and/or the activation of Wnt signaling is induced, for 20 days or 21 days. In certain embodiments, the cells are contacted with or exposed to the at least one Wnt activator, and/or the activation of Wnt signaling is induced, from day 0 through about day 20. In certain embodiments, the at least one Wnt activator is added every day or every other day to a cell culture medium comprising the stem cells from day 0 through about day 20. In certain embodiments, the at least one Wnt activator is added every day (daily) to a cell culture medium comprising the stem cells from day 0 to about day 20. In certain embodiments, the at least one Wnt activator comprises CHIR99021.

In certain embodiments, the concentration of the Wnt activator contacted with or exposed to the cells is between about 0.5 μM and about 15 μM, or between about 0.5 μM and about 10 μM, or between about 0.5 μM and about 5 μM, or between about 0.5 and about 3 μM, or between about 1 μM and about 15 μM, or between about 1 μM and about 10 μM, or between about 1 μM and about 5 μM. In certain embodiments, the concentration of the Wnt activator contacted with or exposed to the cells is between about 1 μM and about 5 μM, or between about 0.5 and about 3 μM. In certain embodiments, the concentration of the Wnt activator contacted with or exposed to the cells is about 1.5 μM. In certain embodiments, the concentration of the Wnt activator contacted with or exposed to the cells is about 3 μM. In certain embodiments, the concentration of the Wnt activator contacted with or exposed to the cells is about 3 μM for about 3 days (e.g., 3 days, 4 days, or 5 days, e.g., from day 0 to day 3, or from day 0 to day 4). In certain embodiments, the concentration of the Wnt activator contacted with or exposed to the cells is about 1.5 μM for about 15 days (e.g., 15 days, 16 days or 17 days, e.g., from day 4 to day 20 or from day 5 to day 20). In certain embodiments, the Wnt activator comprises CHIR99021.

### 5.2.1.2. FGF Activation and FGF Activators

In certain embodiments, the methods disclosed herein comprise inducing activation of FGF signaling in the cells. In certain embodiments, the cells are contacted with at least one FGF activator to induce activation of FGF signaling.

In certain embodiments, the at least one activator of FGF signaling is a member of FGF1 subfamily, FGF4 subfamily, or FGF8 subfamily. In certain embodiments, the at least one FGF activator is selected from the group consisting of FGF1, FGF2, FGF4, FGF6, FGF7, FGF8, FGF17, FGF18, derivatives thereof, and combination thereof. In certain embodiments, the at least one FGF activator comprises FGF2.

In certain embodiments, the stem cells are exposed to or contacted with the at least one FGF activator, and or the activation of FGF signaling are induce, for at least about 1 day. In certain embodiments, the stem cells are contacted with or exposed to the at least one FGF activator, and/or the activation of FGF signaling are induce, for up to about 10 days (e.g., 8 days). In certain embodiments, the stem cells are contacted with or exposed to the at least one FGF activator, and/or the activation of FGF signaling are induce, for between about 1 days and about 5 days. In certain embodiments, the stem cells are contacted with or exposed to the at least one FGF activator, and/or the activation of FGF signaling are induce, for about 1 day, or about 5 days. In certain embodiments, the stem cells are contacted with or exposed to the at least one FGF activator for 1 day, 2 days, 3 days, 4 days, or 5 days. In certain embodiments, the stein cells are contacted with or exposed to the at least one FGF activator, and/or the activation of FGF signaling are induce, for about 3 days. In certain embodiments, the stem cells are contacted with or exposed to the at least one FGF activator, and/or the activation of FGF signaling are induce, for 3 days. In certain embodiments, the stem cells are contacted with or exposed to the at least one FGF activator, and/or the activation of FGF signaling are induce, for 4 days. In certain embodiments, the stem cells are contacted with or exposed to the at least one FGF activator, and/or the activation of FGF signaling are induce, for 5 days. In certain embodiments, the cells are contacted with or exposed to the at least one FGF activator, and/or the activation of FGF signaling are induce, from day 0 through about day 3. In certain embodiments, the cells are contacted with or exposed to the at least one FGF activator, and/or the activation of FGF signaling are induce, from day 0 through about day 4. In certain embodiments, the at least one FGF activator is added every day or every other day to a cell culture medium comprising the stem cells from day 0 through about day 3. In certain embodiments, the at least one FGF activator is added every day or every other day to a cell culture medium comprising the stein cells from day 0 through about day 4. In certain embodiments, the at least one FGF activator is added every day (daily) to a cell culture medium comprising the stem cells from day 0 to about day 3. In certain embodiments, the at least one FGF activator is added every day (daily) to a cell culture medium comprising the stem cells from day 0 to about day 4.

In certain embodiments, the concentration of the at least one FGF activator contacted with or exposed to the cells is between about 10 ng/ml and about 200 ng/ml, between about 10 ng/ml and about 100 ng/ml, between about 10 ng/ml and about 150 ng/ml, between about 100 ng/ml and about 150 ng/ml, between about 50 ng/ml and about 100 ng/ml, between about 50 ng/ml and about 150 ng/ml, between about 50 ng/ml and about 200 ng/ml, or between about 150 ng/ml and about 200 ng/ml. In certain embodiments, the concentration of the at least one FGF activator contacted with or exposed to the cells is between about 50 ng/ml and about 150 ng/ml. In certain embodiments, the concentration of the at least one FGF activator contacted with or exposed to the cells is about 100 ng/ml.

In certain embodiments, the contact of the cells with the at least one activator of FGF signaling is initiated on the same day as the initial contact of the cell with the at least one activator of Wnt signaling. In certain embodiments, the induction of the activation of FGF signaling and the induction of the activation of Wnt signaling inhibition are initiated on the same day.

### 5.2.1.3. Induction of Sacral Neural Crest Patterning and Molecules Therefor

In certain embodiments, the methods disclosed herein comprise inducing sacral neural crest patterning in the cells. In certain embodiments, the cells are contacted with at least one molecule that induces sacral neural crest patterning to induce such sacral neural crest patterning.

In certain embodiments, the at least one molecule that induces sacral neural crest patterning is a member of transforming growth factor β (TGFβ) family. In certain embodiments, the at least one molecule that induces sacral neural crest patterning activates SMAD2 and/or SMAD3. In certain embodiments, the at least one molecule that induces sacral neural crest patterning activates type 1 activin-like receptor kinase receptors ALK4, ALK4, and/or ALK7. In certain embodiments, the at least one molecule that induces sacral neural crest patterning comprises a BMP. In certain embodiments, the at least one molecule that induces sacral neural crest patterning comprises a growth differentiation factor (GDF). Non-limiting examples of molecules that induce sacral neural crest patterning include GDF11, GDF8, and combinations thereof. In certain embodiments, the at least one molecule that induces sacral neural crest patterning comprises GDF11.

GDF11, also known as growth differentiation factor 11 or bone morphogenetic protein 11 (BMP11), is a protein that is encoded by the GDF11 gene. GDF8, also known as growth differentiation factor 8 or myostatin, is a protein that is encoded by the MSTN gene. GDF11 GDF8 are members of the super family of the Transforming Growth Factor β, and are activators of SMAD2:3 and type 1 activin-like receptor kinase receptors ALK4/5/7.

In certain embodiments, the stem cells are exposed to or contacted with the at least one molecule that induces sacral neural crest patterning, and/or the sacral neural crest patterning is induced, for at least about 1 day. In certain embodiments, the stem cells are contacted with or exposed to the at least one molecule that induces sacral neural crest patterning, and/or the sacral neural crest patterning is induced, for up to about 20 days. In certain embodiments, the stem cells are contacted with or exposed to the at least one molecule that induces sacral neural crest patterning, and/or the sacral neural crest patterning is induced, for between about 1 day and about 5 days. In certain embodiments, the stem cells are contacted with or exposed to the at least one molecule that induces sacral neural crest patterning, and/or the sacral neural crest patterning is induced, for about 1 day, or about 5 days. In certain embodiments, the stem cells are contacted with or exposed to the at least one molecule that induces sacral neural crest patterning, and/or the sacral neural crest patterning is induced, for 1 day, 2 days, 3 days, 4 days, or 5 days. In certain embodiments, the stem cells are contacted with or exposed to the at least one molecule that induces sacral neural crest patterning, and/or the sacral neural crest patterning is induced, for about 3 days. In certain embodiments, the stem cells are contacted with or exposed to the at least one molecule that induces sacral neural crest patterning, and/or the sacral neural crest patterning is induced, for 3 days. In certain embodiments, the stem cells are contacted with or exposed to the at least one molecule that induces sacral neural crest patterning, and/or the sacral neural crest patterning is induced, for 4 days. In certain embodiments, the cells are contacted with or exposed to the at least one molecule that induces sacral neural crest patterning, and/or the sacral neural crest patterning is induced, from day 0 through about day 3, or from day 1 through about day 4. In certain embodiments, the at least one molecule that induces sacral neural crest patterning is added every day or every other day to a cell culture medium comprising the stem cells from day 0 through about day 3, or from day 1 through about day 4. In certain embodiments, the at least one molecule that induces sacral neural crest patterning is added every day (daily) to a cell culture medium comprising the stem cells from day 0 to about day 3, or from day 1 through about day 4.

In certain embodiments, the concentration of the at least one molecule that induces sacral neural crest patterning contacted with or exposed to the cells is between about 10 ng/ml and about 100 ng/ml, between about 10 ng/ml and about 50 ng/ml, between about 10 ng/ml and about 80 ng/ml, between about 50 ng/ml and about 80 ng/ml, between about 30 ng/ml and about 50 ng/ml, between about 30 ng/ml and about 80 ng/ml, between about 30 ng/ml and about 100 ng/ml, or between about 80 ng/ml and about 100 ng/ml. In certain embodiments, the concentration of the at least one molecule that induces sacral neural crest patterning contacted with or exposed to the cells is between about 30 ng/ml and about 80 ng/ml. In certain embodiments, the concentration of the at least one molecule that induces sacral neural crest patterning contacted with or exposed to the cells is about 50 ng/ml. In certain embodiments, the at least one molecule that induces sacral neural crest patterning comprises GDF11.

In certain embodiments, the contact of the cells with the at least one molecule that induces sacral neural crest patterning is not initiated on the same day as the initial contact of the cell with the at least one activator of Wnt signaling. In certain embodiments, the contact of the cells with the at least one molecule that induces sacral neural crest patterning is initiated after the initial contact of the cells with the at least one activator of Wnt signaling.

In certain embodiments, the contact of the cells with the at least one molecule that induces sacral neural crest patterning is initiated on the same day as the initial contact of the cell with the at least one activator of Wnt signaling. In certain embodiments, the induction of sacral neural crest patterning the induction of the activation of Wnt signaling are initiated on the same day.

In certain embodiments, the contact of the cells with the at least one molecule that induces sacral neural crest patterning is not initiated on the same day as the initial contact of the cell with the at least one FGF activator. In certain embodiments, the contact of the cells with the at least one molecule that induces sacral neural crest patterning is initiated after the initial contact of the cells with the at least one FGF activator.

In certain embodiments, the contact of the cells with the at least one FGF activator is initiated on the same day as the at least one molecule that induces sacral neural crest patterning. In certain embodiments, the induction of the activation of FGF signaling and the induction of the sacral neural crest patterning are initiated on the same day.

### 5.2.1.1. SMAD Inhibition and SMAD Inhibitors

In certain embodiments, the methods disclosed herein further comprise inducing inhibition of SMAD signaling in the cells. In certain embodiments, the cells are contacted with at least one SMAD inhibitor to induce inhibition of SMAD signaling.

In certain embodiments, the inhibition of SMAD signaling comprises inducing inhibition of transforming growth factor beta (TGFβ)/Activin-Nodal signaling in the cells. In certain embodiments, the inhibition of SMAD signaling further comprises inducing inhibition of BMP signaling in the cells. Non-limiting examples of SMAD inhibitors include inhibitors of TGFβ/Activin-Nodal signaling (referred to as “TGFβ/Activin-Nodal inhibitor”), and inhibitors of BMP signaling (referred to as “BMP inhibitor”).

In certain embodiments, the at least one SMAD inhibitor comprises a TGFβ/Activin-Nodal inhibitor. In certain embodiments, the TGFβ/Activin-Nodal inhibitor can neutralize the ligands including TGFβs, BMPs, Nodal, and activins, and/or block their signal pathways through blocking the receptors and downstream effectors. Non-limiting examples of TGFβ/Activin-Nodal inhibitors include those disclosed in WO/2010/096496, WO/2011/149762, WO/2013/067362, WO2014/176606, WO2015/077648, Chambers et al., Nat Biotechnol. 2009 March; 27(3):275-80, Kriks et al., Nature. 2011 Nov. 6; 480(7378):547-51, and Chambers et al., Nat Biotechnol. 2012 Jul. 1; 30(7):715-20 (2012), all of which are incorporated by reference in their entireties herein for all purposes. In certain embodiments, the at least one TGFβ/Activin-Nodal inhibitor is selected from inhibitors of ALK5, inhibitors of ALK4, inhibitors of ALK7, and combinations thereof). In certain embodiments, the TGFβ/Activin-Nodal inhibitor comprises an inhibitor of ALK5. In certain embodiments, the TGFβ/Activin-Nodal inhibitor is a small molecule selected from SB431542, derivatives thereof, and mixtures thereof. “SB431542” refers to a molecule with a number CAS 301836-41-9, a molecular formula of C₂₂H₁₈N₄O₃, and a name of 4-[4-(1,3-benzodioxol-5-yl)-5-(2-pyridinyl)-1H-imidazol-2-yl]-benzamide, for example, see structure below:

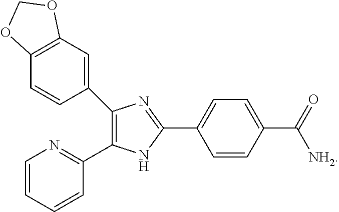 [SMILES: NC(=O)c1ccc(-c2nc(-c3ccc4c(c3)OCO4)c(-c3ccccn3)[nH]2)cc1]

In certain embodiments, the TGFβ/Activin-Nodal inhibitor comprises SB431542. In certain embodiments, the TGFβ/Activin-Nodal inhibitor comprises a derivative of SB431542. In certain embodiments, the derivative of SB431542 is A83-01. In certain embodiments, the derivative of SB431542 is RepSox.

In certain embodiments, the at least one SMAD inhibitor comprises a BMP inhibitor. Non-limiting examples of BMP inhibitors include those disclosed in WO2011/149762, Chambers et al., Nat Biotechnol. 2009 March; 27(3):275-80. Kriks et al., Nature. 2011 Nov. 6; 480(7378):547-51, and Chambers et al., Nat Biotechnol. 2012 Jul. 1; 30(7):715-20, all of which are incorporated by reference in their entireties. In certain embodiments, the BMP inhibitor is a small molecule selected from LDN193189, Noggin, dorsomorphin, derivatives thereof, and mixtures thereof. “LDN193189” refers to a small molecule DM-3189, IUPAC name 4-(6-(4-(piperazin-1-yl)phenyl)pyrazolo[1,5-a]pyrimidin-3-yl)quinoline, with a chemical formula of C₂₅H₂₂N₆ with the following formula.

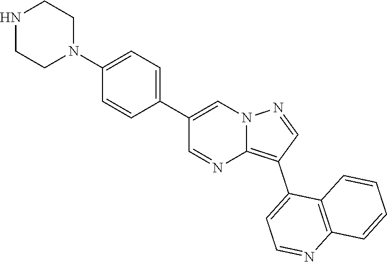 [SMILES: c1ccc2c(-c3cnn4cc(-c5ccc(N6CCNCC6)cc5)cnc34)ccnc2c1]

LDN193189 is capable of functioning as a SMAD signaling inhibitor. LDN193189 is also highly potent small-molecule inhibitor of ALK2, ALK3, and ALK6, protein tyrosine kinases (PTK), inhibiting signaling of members of the ALK1 and ALK3 families of type 1 TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smad1, Smad5, and Smad8 (Yu et al. (2008) Nat Med 14:1363-1369; Cuny et al. (2008) Bioorg. Med. Chem. Lett. 18: 4388-4392, herein incorporated by reference).

In certain embodiments, the BMP inhibitor comprises LDN193189. In certain embodiments, the BMP inhibitor comprises Noggin.

In certain embodiments, the stem cells are exposed to one SMAD inhibitor, e.g., one TGFβ/Activin-Nodal inhibitor. In certain embodiments, the TGFβ/Activin-Nodal inhibitor is SB431542. In certain embodiments, the TGFβ/Activin-Nodal inhibitor is a SB431542 derivative. In certain embodiments, the TGFβ/Activin-Nodal inhibitor is A83-01. In certain embodiments, the TGFβ/Activin-Nodal inhibitor is RepSox.

In certain embodiments, the stem cells are exposed to two SMAD inhibitors. In certain embodiments, the two SMAD inhibitors are a TGFβ/Activin-Nodal inhibitor and a BMP inhibitor. In certain embodiments, the stem cells are exposed to SB431542, A83-01, or RepSox, and LDN193189 or Noggin. In certain embodiments, the stem cells are exposed to SB431542 and LDN193189.

In certain embodiments, the stem cells are exposed to or contacted with at least one SMAD inhibitor, and/or the inhibition of SMAD signaling is induced, for at least about 1 day. In certain embodiments, the stem cells are contacted with or exposed to the at least one SMAD inhibitor, and/or the inhibition of SMAD signaling is induced, for up to about 20 days. In certain embodiments, the stem cells are contacted with or exposed to the at least one SMAD inhibitor, and/or the inhibition of SMAD signaling is induced, for between about 15 days and about 20 days. In certain embodiments, the stem cells are contacted with or exposed to the at least one SMAD inhibitor, and/or the inhibition of SMAD signaling is induced for about 15 days, or about 20 days. In certain embodiments, the stem cells are contacted with or exposed to the at least one SMAD inhibitor, and/or the inhibition of SMAD signaling is induced, for 15 days, 16 days, 17 days, 18 days, 19 days, 20 days. In certain embodiments, the stem cells are contacted with or exposed to the at least one SMAD inhibitor, and/or the inhibition of SMAD signaling is induced, for about 15 days. In certain embodiments, the stem cells are contacted with or exposed to the at least one SMAD inhibitor, and/or the inhibition of SMAD signaling is induced, for about 20 days. In certain embodiments, the stem cells are contacted with or exposed to the at least one SMAD inhibitor, and/or the inhibition of SMAD signaling is induced, for 16 days, 17 days, or 18 days. In certain embodiments, the cells are contacted with or exposed to the at least one SMAD inhibitor, and/or the inhibition of SMAD signaling is induced, from day 0 through day 1 and from day 5 through day 20. In certain embodiments, the cells are contacted with or exposed to the at least one SMAD inhibitor, and/or the inhibition of SMAD signaling is induced, from day 4 through day 20.

In certain embodiments, the contact of the cells with the one SMAD inhibitor is not imitated on the same day as the initial contact of the cells with the at least one molecule that induces sacral neural crest patterning, and/or the induction of the inhibition of SMAD signaling is not initiated on the same day as the induction of sacral neural crest patterning. In certain embodiments, the contact of the cells with the at least one molecule that induces sacral neural crest patterning is initiated after the initial contact of the cell with the at least one SMAD inhibitor, and/or the induction of sacral neural crest patterning is initiated after the initial induction of the SMAD inhibitor.

In certain embodiments, the contact of the cells with the one SMAD inhibitor is imitated on the same day as the initial contact of the cells with the at least one Wnt activator, and/or the induction of the inhibition of SMAD signaling is initiated on the same day as the induction of the activation of Wnt signaling.

In certain embodiments, the at least one SMAD inhibitor is added every day or every other day to a cell culture medium comprising the stem cells from day 0 through day 1 and from day 5 through day 20. In certain embodiments, the at least one SMAD inhibitor is added every day (daily) to a cell culture medium comprising the stem cells from day 0 through day 1 and from day 5 through day 20. In certain embodiments, the at least one SMAD inhibitor is added every day or every other day to a cell culture medium comprising the stem cells from day 4 through day 20. In certain embodiments, the at least one SMAD inhibitor is added every day (daily) to a cell culture medium comprising the stem cells from day 4 through day 20.

In certain embodiments, the cells are contacted with or exposed to a TGFβ/Activin-Nodal inhibitor. In certain embodiments, the inhibition of SMAD signaling comprises inducing inhibition of TGFβ/Activin-Nodal signaling. In certain embodiments, the concentration of the TGFβ/Activin-Nodal inhibitor contacted with or exposed to the cells is between about 1 μM and about 20 μM, between about 1 μM and about 10 μM, between about 1 μM and about 15 μM, between about 10 μM and about 15 μM, between about 5 μM and about 10 μM, between about 5 μM and about 15 μM, between about 5 μM and about 20 μM. or between about 15 μM and about 20 μM. In certain embodiments, the concentration of the TGFβ/Activin-Nodal inhibitor contacted with or exposed to the cells is between about 1 μM and about 10 μM. In certain embodiments, the concentration of the TGFβ/Activin-Nodal inhibitor contacted with or exposed to the cells is about 2 μM, or about 10 μM. In certain embodiments, the concentration of the TGFβ/Activin-Nodal inhibitor contacted with or exposed to the cells is about 2 μM. In certain embodiments, the concentration of the TGFβ/Activin-Nodal inhibitor contacted with or exposed to the cells is about 2 μM for up to about 3 days. e.g., about 1 day (e.g., 1 day or 2 days, e.g., from day 0 to day). In certain embodiments, the concentration of the TGFβ/Activin-Nodal inhibitor contacted with or exposed to the cells is about 10 μM. In certain embodiments, the concentration of the TGFβ/Activin-Nodal inhibitor contacted with or exposed to the cells is about 10 μM for about 15 days (e.g., 15 days, 16 days or 17 days, e.g., from day 4 to day 20 or from day 5 to day 20). In certain embodiments, the TGFβ/Activin-Nodal inhibitor comprises SB431542 or a derivative thereof (e.g., A83-01, RepSox). In certain embodiments, the TGFβ/Activin-Nodal inhibitor comprises SB431542.

In certain embodiments, the cells are contacted with or exposed to a BMP inhibitor. In certain embodiments, the inhibition of SMAD signaling comprises inducing inhibition of BMP signaling in the cells. In certain embodiments, the concentration of the BMP inhibitor contacted with or exposed to the cells is between about 50 nM and about 500 nM, or between about 100 nM and about 500 nM, or between about 200 nM and about 500 nM, or between about 200 and about 300 nM, or between about 200 nM and about 400 nM, or between about 100 nM and about 250 nM, or between about 100 nM and about 250 nM, or between about 200 nM and about 250 nM, or between about 250 nM and about 300 nM. In certain embodiments, the concentration of the BMP inhibitor contacted with or exposed to the cells is between about 200 nM and about 300 mM. In certain embodiments, the concentration of the BMP inhibitor contacted with or exposed to the cells is about 150 nM, about 200 nM, about 250 nM, about 300 nM, or about 350 nM. In certain embodiments, the concentration of the BMP inhibitor contacted with or exposed to the cells is about 250 nM. In certain embodiments, the BMP inhibitor comprises LDN193189 or a derivative thereof. In certain embodiments, the BMP inhibitor comprises LDN193189.

In certain embodiments, the cells are contacted with or exposed to the TGFβ/Activin-Nodal inhibitor and the BMP inhibitor simultaneously. In certain embodiments, the inhibition of TGFβ/Activin-Nodal signaling and the inhibition of BMP signaling are induced in the cells simultaneously. In certain embodiments, the stem cells are contacted with or exposed to the TGFβ/Activin-Nodal inhibitor and the BMP inhibitor, and/or the inhibition of TGFβ/Activin-Nodal signaling and the inhibition of BMP signaling are induced, for about 15 days. In certain embodiments, the stem cells are contacted with or exposed to the TGFβ/Activin-Nodal inhibitor and the BMP inhibitor, and/or the inhibition of TGFβ/Activin-Nodal signaling and the inhibition of BMP signaling are induced, for about 20 days (e.g., for 15 days, 16 days, 17 days, or for 18 days). In certain embodiments, the cells are contacted with or exposed to the TGFβ/Activin-Nodal inhibitor and the BMP inhibitor, and/or the inhibition of TGFβ/Activin-Nodal signaling and the inhibition of BMP signaling are induced, from day 0 through day 1 and from day 5 through about day 20. In certain embodiments, the cells are contacted with or exposed to the TGFβ/Activin-Nodal inhibitor and the BMP inhibitor, and/or the inhibition of TGFβ/Activin-Nodal signaling and the inhibition of BMP signaling are induced, from day 4 through about day 20. In certain embodiments, the TGFβ/Activin-Nodal inhibitor and the BMP inhibitor are added every day or every other day to a cell culture medium comprising the stem cells from day 0 through day 1 and from day 5 through about day 20. In certain embodiments, the TGFβ/Activin-Nodal inhibitor and the BMP inhibitor are added every day or every other day to a cell culture medium comprising the stem cells from day 4 through about day 20. In certain embodiments, the TGFβ/Activin-Nodal inhibitor and the BMP inhibitor are added every day (daily) to a cell culture medium comprising the stem cells from day 0 through day 1 and from day 5 through about day 20. In certain embodiments, the TGFβ/Activin-Nodal inhibitor and the BMP inhibitor are added every day (daily) to a cell culture medium comprising the stem cells from 4 through about day 20.

### 5.2.1.2. BMP

In certain embodiments, the method comprises inducing activation of BMP signaling in the cells. In certain embodiments, the stem cells are further contacted with at least a BMP. In certain embodiments, the method comprises inducing activation of BMP signaling and inhibition of TGFβ/Activin-Nodal signaling in the cells. In certain embodiments, the stem cells are exposed to a TGFβ/Activin-Nodal inhibitor and a BMP.

Non-limiting examples of BMP include BMP1, BMP2, BMP3, BMP4, BMP5, BMP6, BMP7, BMP8a, BMP8b, BMP10, BMP11, BMP15, and combinations thereof.

In certain embodiments, the stem cells are exposed to or contacted with the at least one BMP, and/or the inhibition of BMP signaling is induced, for at least about 1 day. In certain embodiments, the stem cells are contacted with or exposed to the at least one BMP, and/or the inhibition of BMP signaling is induced, for up to about 25 days. In certain embodiments, the stein cells are contacted with or exposed to the at least one BMP, and/or the inhibition of BMP signaling is induced, for between about 15 days and about 25 days. In certain embodiments, the stem cells are contacted with or exposed to the at least one BMP, and/or the inhibition of BMP signaling is induced, for about 15 days, about 20 days, or about 25 days. In certain embodiments, the stem cells are contacted with or exposed to the at least one BMP, and/or the inhibition of BMP signaling is induced, for 15 days, 16 days, 17 days, 18 days, 19 days, 20 days, or 21 days. In certain embodiments, the stem cells are contacted with or exposed to the at least one BMP, and/or the inhibition of BMP signaling is induced, for about 20 days. In certain embodiments, the stem cells are contacted with or exposed to the at least one BMP, and/or the inhibition of BMP signaling is induced, for 20 days or 21 days. In certain embodiments, the cells are contacted with or exposed to the at least one BMP, and/or the inhibition of BMP signaling is induced, from day 0 through about day 20. In certain embodiments, the at least one BMP is added every day or every other day to a cell culture medium comprising the stem cells from day 0 through about day 20. In certain embodiments, the at least one BMP is added every day (daily) to a cell culture medium comprising the stem cells from day 0 to about day 20. In certain embodiments, the at least one BMP comprises BMP4.

In certain embodiments, the concentration of the BMP contacted with or exposed to the cells is between about 0.1 ng/ml and about 2 ng/ml, between about 0.1 ng/ml and about 1 ng/ml, between about 0.1 ng/ml and about 1.5 ng/ml, between about 1 ng/ml and about 1.5 ng/ml, between about 0.5 ng/ml and about 1 ng/ml, between about 0.5 ng/ml and about 1.5 ng/ml, between about 0.5 ng/ml and about 2 ng/ml, or between about 1.5 ng/ml and about 2 ng/ml. In certain embodiments, the concentration of the BMP contacted with or exposed to the cells is between about 0.5 ng/ml and about 1.5 ng/ml. In certain embodiments, the concentration of the BMP contacted with or exposed to the cells is about 1 ng/ml. In certain embodiments, the BMP comprises BMP4.

In certain embodiments, the cells are contacted with or exposed to the Wnt activator and the BMP simultaneously. In certain embodiments, the activation of Wnt signaling and the activation of BMP signaling are induced in the cells simultaneously. In certain embodiments, the stem cells are contacted with or exposed to the Wnt activator and the BMP, and/or the activation of Wnt signaling activation of BMP signaling are induced, for about 20 days (e.g., for 20 days or for 21 days). In certain embodiments, the cells are contacted with or exposed to the Wnt activator and the BMP, and/or the activation of Wnt signaling and the activation of BMP signaling are induced, from day 0 through about day 20. In certain embodiments, the Wnt activator and the BMP are added every day or every other day to a cell culture medium comprising the stem cells from day 0 through about day 20. In certain embodiments, the Wnt activator and the BMP are added every day (daily) to a cell culture medium comprising the stem cells from day 0 to about day 20.

### 5.2.1.6. EXEMPLARY METHODS

### Exemplary Method A

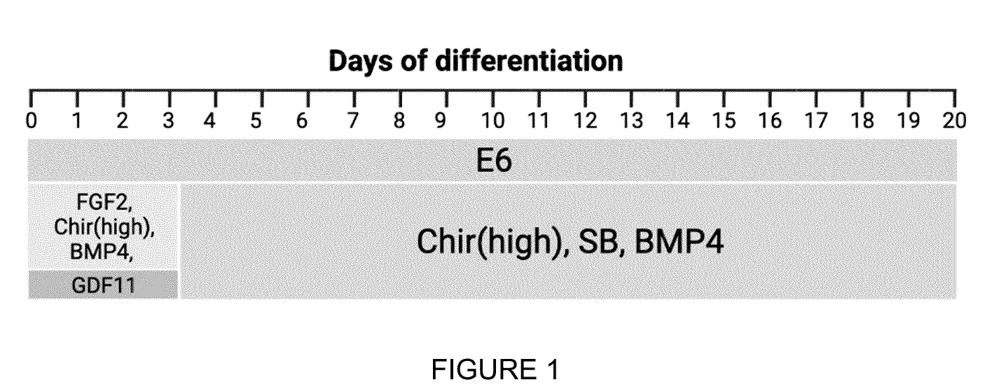

In certain embodiments, the presently disclosed methods for inducing in vitro differentiation of stem cells into cells expressing at least one marker indicating a sacral neural crest lineage comprise contacting the stem cells with at least one inhibitor of SMAD signaling (e.g., SB431542, e.g., at a concentration of about 10 PM) for about 15 days (e.g., 16 days or 17 days, e.g., from day 4 through day 20), at least one BMP (e.g., BMP4, e.g., at a concentration of about 1 ng/ml) for about 20 days (e.g., 20 days or 21 days, e.g., from day 0 to day 20), at least one activator of Wnt signaling (e.g., CHIR99021) for about 20 days (e.g., 20 days or 21 days, e.g., from day 0 to day 20) (wherein the concentration of the at least one activator of Wnt signaling contacted with the cells is about 3 μM for about 3 days (e.g., 3 days, or 4 days, e.g., from day 0 to day 3), and the concentration of at least one activator of Wnt signaling is about 1.5 μM for about 15 days (e.g., 16 days or 17 days, e.g., from day 4 to day 20)), at least one activator of FGF signaling (e.g., FGF2, e.g., at a concentration of about 100 ng/ml) for about 3 days (e.g., 3 days, or 4 days, e.g., from day 0 to day 3), and at least one molecule that induces sacral neural crest patterning (e.g., GDF11, e.g., at a concentration of about 50 ng/ml) for about 3 days (e.g., 3 days or 4 days, e.g., from day 0 to day 3)). See FIG. 1 and Example 1.

### Exemplary Method B

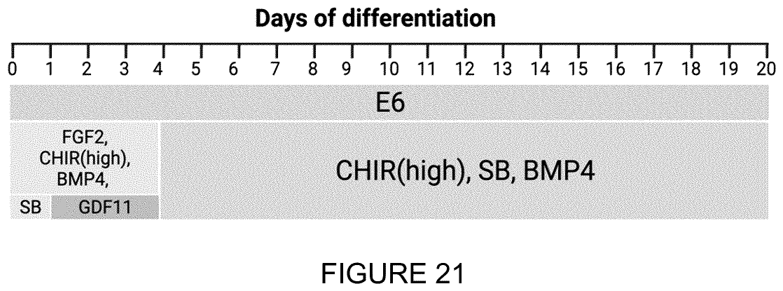

In certain embodiments, the presently disclosed methods for inducing in vitro differentiation of stem cells into cells expressing at least one marker indicating a sacral neural crest lineage comprise contacting the stem cells with at least one inhibitor of SMAD signaling (e.g., SB431542) for about 15 days (e.g., 16 days, 17 days, or 18 days, e.g., from day 0 to day 1 and from day 5 to day 20 (wherein the concentration of at least one inhibitor of SMAD signaling is about 10 μM for about 15 days (e.g., 15 days or 16 days, e.g., from day 5 through day 20), and the concentration of the at least one inhibitor of SMAD signaling is about 2 μM for about 1 day (e.g., 1 day or 2 days), at least one BMP (e.g., BMP4, e.g., at a concentration of about 1 ng/ml) for about 20 days (e.g., 20 days or 21 days, e.g., from day 0 to day 20), at least one activator of Wnt signaling (e.g., CHIR99021) for about 20 days (e.g., 20 days or 21 days, e.g., from day 0 to day 20) (wherein the concentration of the at least one activator of Wnt signaling contacted with the cells is about 3 μM for about 5 days (e.g., 4 days, or 5 days, e.g., from day 0 to day 4), and the concentration of at least one activator of Wnt signaling is about 1.5 μM for about 15 days (e.g., 15 days or 16 days, e.g., from day 5 to day 20)), at least one activator of FGF signaling (e.g., FGF2, e.g., at a concentration of about 100 ng/ml) for about 5 days (e.g., 4 days, or 5 days, e.g., from day 0 to day 4), and at least one molecule that induces sacral neural crest patterning (e.g., GDF11, e.g., at a concentration of about 50 ng/ml) for about 3 days (e.g., 3 days or 4 days, e.g., from day 1 to day 4)). See FIG. 21 and Example 7.

### 5.2.2. In Vitro Induction of Sacral Neural Crest Lineage Cells to Enteric Neurons and Enteric Glia

The sacral neural crest lineage cells can be further induced/matured in vitro to enteric neurons or enteric glia cells. Enteric neurons can be immature enteric neurons, mature enteric neurons, or a combination thereof. Enteric glia cells can be immature enteric glia cells, mature enteric glia cells, or a combination thereof. The differentiated sacral neural crest lineage cells disclosed herein can be subjected to conditions favoring maturation of sacral neural crest lineage cells into a population of enteric neurons or a population of enteric glia cells.

In certain embodiments, the conditions favoring maturation (e.g., maturation to enteric neurons or enteric glia cells) comprise culturing the differentiated sacral neural crest lineage cells in a suitable cell culture medium. In certain embodiments, the suitable cell culture medium is a Neurobasal® medium (NB). In certain embodiments, the suitable cell culture medium is an NB medium supplemented with L-Glutamine (e.g., from Gibco, 25030-164), N2 (e.g., from Stem Cell Technologies, 07156), and B27 (e.g., from Life Technologies, 17504044). The differentiated sacral neural crest lineage cells can be cultured in the suitable cell culture medium for at least about 1 day, for at least about 5 days, for at least about 10 days, for at least about 15 days, for at least about 20 days, for at least about 25 days, for at least about 30 days, for at least about 35 days, for at least about 40 days, for at least about 45 days, or for at least about 50 days, to produce enteric neurons or enteric glia cells. In certain embodiments, the differentiated sacral neural crest lineage cells can be cultured in the suitable cell culture medium for 1 day, for at least 2 days, for at least 3 days, for at least 4 days, for at least 5 days, for at least 6 days, for at least 7 days, for at least 8 days, for at least 9 days, for at least 10 days, for at least 1 days, for at least 12 days, for at least 13 days, for at least 14 days, for at least 15 days, for at least 16 days, for at least 17 days, for at least 18 days, for at least 19 days, for at least 20 days, for at least 25 days, for at least 30 days, for at least 35 days, for at least 40 days, for at least 45 days, or for at least 50 days, to produce enteric neurons or enteric glia cells.

In certain embodiments, the suitable cell culture medium comprises at least one molecule that enhances maturation of sacral neural crest lineage cells to enteric neurons or enteric glia cells. In certain embodiments, the conditions favoring maturation comprises enhancing maturation of sacral neural crest lineage cells to enteric neurons or enteric glia cells. In certain embodiments, the conditions favoring maturation comprises contacting the differentiated sacral neural crest lineage cells with at least one molecule that enhances maturation of sacral neural crest lineage cells to enteric neurons or enteric glia cells. In certain embodiments, the conditions favoring maturation comprises enhancing maturation of sacral neural crest lineage cells to enteric neurons or enteric glia cells. In certain embodiments, enhancing maturation of sacral neural crest lineage cells to enteric neurons or enteric glia cells comprises inducing cell growth and inducing activation of Wnt signaling. In certain embodiments, the at least one molecule that enhances maturation of sacral neural crest lineage cells to enteric neurons or enteric glia cells is selected from the group consisting of growth factors and Wnt activators described herein. In certain embodiments, the induction of cell growth comprises inducing activation of FGF signaling. Non-limiting examples of growth factors include FGF activators, glial cell line derived neurotrophic factor (GDNF), and ascorbic acid. In certain embodiments, the conditions comprise inducing activation of FGF signaling and activation of Wnt signaling in the cells. In certain embodiments, the differentiated sacral neural crest lineage cells are contacted with at least one FGF activator and at least one Wnt activator to produce a population of enteric neurons or enteric glia cells. In certain embodiments, the suitable cell culture medium comprises at least one FGF activator and at least one Wnt activator. Non-limiting examples of activators of FGF signaling include FGF2, FGF4, FGF7, and FGF8. In certain embodiments, the at least one FGF activator is FGF2. In certain embodiments, the at least one Wnt activator is CHIR99021.

In certain embodiments, the sacral neural crest lineage cells are contacted with the at least one FGF activator and at least one Wnt activator, and/or the activation of FGF signaling and the activation of Wnt signaling are induced, for at least about 1 day, at least about 5 days, or at least about 10 days, to produce enteric neurons or enteric glia cells. In certain embodiments, the sacral neural crest lineage cells are contacted with the at least one FGF activator and at least one Wnt activator for at least 1 day, at least 2 days, at least 3 days, at least 4 days, at least 5 days, at least 6 days, at least 7 days, at least 8 days, at least 9 days, or at least 10 days, to produce enteric neurons or enteric glia cells, in certain embodiments, the sacral neural crest lineage cells are contacted with the at least one FGF activator and at least one Wnt activator, and/or the activation of FGF signaling and the activation of Wnt signaling are induced, for between about 1 day and about 10 days, between about 1 day and about 5 days, between about 5 days and about 10 days, to produce enteric neurons or enteric glia cells. In certain embodiments, the sacral neural crest lineage cells are contacted with the at least one FGF activator and at least one Wnt activator, and/or the activation of FGF signaling and the activation of Wnt signaling are induced, for between about 1 day and about 5 days to produce enteric neurons or enteric glia cells. In certain embodiments, the sacral neural crest lineage cells are contacted with the at least one FGF activator and at least one Wnt activator, and/or the activation of FGF signaling and the activation of Wnt signaling are induced, for about 4 days to produce enteric neurons or enteric glia cells.

In certain embodiments, the sacral neural crest lineage cells are contacted with the at least one activator of FGF signaling in a concentration of from about 1 nM to 100 nM, from about 1 nM to 20 nM, from about 1 nM to 15 nM, from about 1 nM to 10 nM, from about 1 nM to 5 nM, from about 5 nM to 10 nM, from about 5 nM to 15 nM, from about 15 nM to 20 nM, from about 20 nM to 30 nM, from about 30 nM to 40 nM, from about 40 nM to 50 nM, from about 50 nM to 60 nM, from about 60 nM to 70 nM, from about 70 nM to 80 nM, from about 80 nM to 90 nM, or from about 90 nM to 100 nM, to produce enteric neurons or enteric glia cells. In certain embodiments, the sacral neural crest lineage cells are contacted with the at least one activator of FGF signaling in a concentration of from about from about 5 nM to 15 nM to produce enteric neurons or enteric glia cells. In certain embodiments, the sacral neural crest lineage cells are contacted with the at least one activator of FGF signaling in a concentration of about 10 nM to produce enteric neurons or enteric glia cells. In certain embodiments, the sacral neural crest lineage cells are contacted with the at least one activator of FGF signaling in any one of the above-described concentrations daily, every other day or every two days to produce enteric neurons or enteric glia cells. In certain embodiments, the sacral neural crest lineage cells are contacted with the at least one activator of FGF signaling in a concentration of about 10 nM daily to produce enteric neurons or enteric glia cells.

In certain embodiments, the sacral neural crest lineage cells are contacted with the at least one Wnt activator in a concentration of from about 1 μM to 100 μM, from about 1 μM to 20 μM, from about 1 μM to 15 μM, from about 1 μM to 10 μM, from about 1 μM to 5 μM, from about 5 μM to 10 μM, from about 5 μM to 15 μM, from about 15 μM to 20 μM, from about 20 μM to 30 μM, from about 30 μM to 40 μM, from about 40 μM to 50 μM, from about 50 μM to 60 μM, from about 60 μM to 70 μM, from about 70 μM to 80 μM, from about 80 μM to 90 μM, or from about 90 μM to 100 μM, to produce enteric neurons or enteric glia cells. In certain embodiments, the sacral neural crest lineage cells are contacted with the at least one Wnt activator in a concentration of from about from about 1 μM to 5 μM to produce enteric neurons or enteric glia cells. In certain embodiments, the sacral neural crest lineage cells are contacted with the at least one Wnt activator in a concentration of about 3 μM to produce enteric neurons or enteric glia cells. In certain embodiments, the sacral neural crest lineage cells are contacted with the at least one Wnt activator in any one of the above-described concentrations daily, every other day or every two days to produce enteric neurons or enteric glia cells. In certain embodiments, the sacral neural crest lineage cells are contacted with the at least one Wnt activator in a concentration of about 3 μM daily to produce enteric neurons or enteric glia cells.

In certain embodiments, the sacral neural crest lineage cells are contacted with the at least one FGF activator and at least one Wnt activator in a cell culture medium to produce enteric neurons or enteric glia cells. In certain embodiments, the cell culture medium is an NB medium. In certain embodiments, the cell culture medium is an NB medium supplemented with L-Glutamine (e.g., from Gibco, 25030-164), N2 (e.g., from Stem Cell Technologies, 07156), and B27 (e.g., from Life Technologies, 17504044).

In certain embodiments, the differentiated sacral neural crest lineage cells are contacted with GDNF and ascorbic acid to produce a population of enteric neurons or enteric glia cells. In certain embodiments, the suitable cell culture medium comprises GDNF and ascorbic acid.

In certain embodiments, the sacral neural crest lineage cells are contacted with GDNF and ascorbic acid for at least about 1 day, at least about 2 days, at least about 3 days, at least about 4 days, at least about 5 days, at least about 6 days, at least about 7 days, at least about 8 days, at least about 9 days, at least about 10 days, at least about 15 days, at least about 20 days, at least about 25 days, at least about 30 days, at least about 35 days, at least about 40 days, at least about 45 days, or at least about 50 days, to produce enteric neurons or enteric glia cells. In certain embodiments, the sacral neural crest lineage cells are contacted with GDNF and ascorbic acid for between about 1 day and about 50 days, between about 1 day and about 10 days, between about 20 days and about 30 days, between about 30 days and about 40 days, or between about 40 days and about 50 days, to produce enteric neurons or enteric glia cells. In certain embodiments, the sacral neural crest lineage cells are contacted with GDNF and ascorbic acid for between about 10 day and about 20 days to produce enteric neurons or enteric glia cells. In certain embodiments, the sacral neural crest lineage cells are contacted with GDNF and ascorbic acid for between about 20 day and about 30 days to produce enteric neurons or enteric glia cells. In certain embodiments, the sacral neural crest lineage cells are contacted with GDNF and ascorbic acid for between about 40 day and about 50 days to produce enteric neurons or enteric glia cells. In certain embodiments, the sacral neural crest lineage cells are contacted with GDNF and ascorbic acid for about 10 days to produce enteric neurons or enteric glia cells. In certain embodiments, the sacral neural crest lineage cells are contacted with GDNF and ascorbic acid for about 25 days to produce enteric neurons or enteric glia cells. In certain embodiments, the sacral neural crest lineage cells are contacted with GDNF and ascorbic acid for about 45 days to produce enteric neurons or enteric glia cells.

In certain embodiments, the sacral neural crest lineage cells are contacted with GDNF in a concentration of from about 1 nM to 100 nM, from about 1 ng/mL to 100 ng/mL, from about 1 ng/mL to 20 ng/mL, from about 20 ng/mL to 30 ng/mL, from about 30 ng/mL to 40 ng/mL, from about 40 ng/mL to 50 ng/mL, from about 50 ng/mL to 60 ng/mL, from about 60 ng/mL to 70 ng/mL, from about 70 ng/mL to 80 ng/mL, from about 80 ng/mL to 90 ng/mL, or from about 90 ng/mL to 100 ng/mL, to produce enteric neurons or enteric glia cells. In certain embodiments, the sacral neural crest lineage cells are contacted with GDNF in a concentration of from about from about 20 ng/mL to 30 ng/mL to produce enteric neurons or enteric glia cells. In certain embodiments, the sacral neural crest lineage cells are contacted with GDNF in a concentration of about 25 ng mL to produce enteric neurons or enteric glia cells. In certain embodiments, the sacral neural crest lineage cells are contacted with GDNF signaling in any one of the above-described concentrations daily, every other day or every two days to produce enteric neurons or enteric glia cells. In certain embodiments, the sacral neural crest lineage cells are contacted with GDNF of FGF signaling in a concentration of about 25 ng/mL daily to produce enteric neurons or enteric glia cells.

In certain embodiments, the sacral neural crest lineage cells are contacted with ascorbic acid in a concentration of from about 50 μM to 200 μM, from about 50 μM to 100 μM, or from about 100 μM to 200 μM, to produce enteric neurons or enteric glia cells. In certain embodiments, the sacral neural crest lineage cells are contacted with ascorbic acid in a concentration of from about from about 50 μM to 200 μM to produce enteric neurons or enteric glia cells. In certain embodiments, the sacral neural crest lineage cells are contacted with ascorbic acid in a concentration of about 100 μM to produce enteric neurons or enteric glia cells. In certain embodiments, the sacral neural crest lineage cells are contacted with ascorbic acid in any one of the above-described concentrations daily, every other day or every two days to produce enteric neurons or enteric glia cells. In certain embodiments, the sacral neural crest lineage cells are contacted with ascorbic acid in a concentration of about 100 μM daily to produce enteric neurons or enteric glia cells.

In certain embodiments, the sacral neural crest lineage cells are contacted with GDNF and ascorbic acid in a cell culture medium to produce enteric neurons or enteric glia cells. In certain embodiments, the cell culture medium is an NB medium. In certain embodiments, the cell culture medium is an NB medium supplemented with L-Glutamine (e.g., from Gibco, 25030-164), N2 (e.g., from Stem Cell Technologies, 07156), and 827 (e.g., from Life Technologies, 17504044).

In certain embodiments, the sacral neural crest lineage cells are contacted with at least one FDF activator and at least one WNT activator, and are subsequently contacted with GDNF and ascorbic acid. In certain embodiments, the sacral neural crest lineage cells are contacted with FGF2 and CHIR990214, and are subsequently contacted with GDNF and ascorbic acid. In certain embodiments, the enteric neurons are immature enteric neurons. In certain embodiments, the immature enteric neurons express at least one enteric neuron marker, including, but not limited to, beta 3 class III tubulin (Tuj1), paired-like homeobox 2A (PHOX2A), paired-like homeobox 2B (PHOX2B), neurotrophic tyrosine kinase receptor type 3 (TRKC), ASCL1, heart and neural crest derivatives expressed 2 (HAND2), and EDNRB.

The immature enteric neurons can further differentiate to mature enteric neurons. In certain embodiments, the enteric neurons are mature enteric neurons. In certain embodiments, the mature enteric neurons express at least one enteric neuron marker, including, but not limited to, 5-hydroxytryptamine (5H-T), gamma-aminobutyric acid (GABA), nitric oxide synthase (NOS), somatostatin (SST), tyrosine hydroxylase (TH), and choline O-acetyltransferase (CHAT).

In certain embodiments, the enteric neurons are a mixture or combination of immature enteric neurons and mature enteric neurons.

In certain embodiments, the enteric glia cells express at least one enteric glia marker, including, but not limited to, GFAP. S100b, vimentin, conexin-43, SOX10, and combinations thereof.

In certain embodiments, the conditions favoring maturation (e.g., maturation to enteric neurons or enteric glia cells) further comprises aggregating the differentiated sacral neural crest lineage cells into 3D spheroids, and culturing the 3D spheroids in suspension culture. In certain embodiments, the 3D spheroids are cultured in suspension culture for about 1 day, about 2 days, about 3 days, about 4 days, about 5 days, about 6 days, about 7 days, about 8 days, about 9 days, or about 10 days. In certain embodiments, the 3D spheroids are cultured in suspension for about 4 days. In certain embodiments, the suspension culture medium is a Neurobasal medium supplemented with N2 supplement, and B27′ supplement comprising CHIR99021 and fibroblast growth factor 2 (FGF2).

In certain embodiments, the conditions favoring maturation (e.g., maturation to enteric neurons or enteric glia cells) further comprises culturing the 3D spheroids in adherent culture in the presence of ascorbic acid (AA) and GDNF for spontaneous differentiation following culturing the 3D spheroids in suspension culture.

The 3D spheroids can be cultured in adherent culture for about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In certain embodiments, the 3D spheroids are cultured in adherent culture for about 3 weeks, e.g., about 20 days. In certain embodiments, the 3D spheroids are cultured in adherent culture for about 6 weeks, e.g., about 40 days. In certain embodiments, the adherent culture medium is a Neurobasal medium supplemented with N2 supplement, and B27® supplement comprising GDNF and ascorbic acid. In certain non-limiting embodiment, the adherent culture is performed on a surface with a suitable coating, for example, poly ornithine, laminin, fibronectin, or a combination thereof (e.g., the methods described in Zeltner et al., (2014), which is incorporated herein by reference in its entirety). In certain embodiments, the cells aggregated to the 3D spheroids first differentiate to a population of immature neurons in the adherent culture and migrate out of the 3D spheroids.

### 5.2.3. Cell Culture Media

In certain embodiments, the above-described inhibitors, activators and molecules are added to a cell culture medium comprising the cells. Suitable cell culture media include, but are not limited to, Knockout® Serum Replacement (“KSR”) medium, Neurobasal, medium (NB), N2 medium, B-27 medium, and Essential 8®/Essential 6® (“E8/E6”) medium, and combinations thereof. KSR medium, NB medium, N2 medium, B-27 medium, and E8/E6 medium are commercially available. KSR medium is a defined, serum-free formulation optimized to grow and maintain undifferentiated hESCs in culture.

In certain embodiments, the cell culture medium is a KSR medium. The components of a KSR medium are disclosed in WO2011/149762. In certain embodiments, a KSR medium comprises Knockout DMEM, Knockout Serum Replacement, L-Glutamine, Pen/Strep, MEM, and 13-mercaptoethanol. In certain embodiments, 1 liter of KSR medium comprises 820 mL of Knockout DMEM, 150 mL of Knockout Serum Replacement, 10 mL of 200 mM L-Glutamine, 10 mL of Pen/Strep, 10 ml, of 10 mM MEM, and 55 μM of 13-mercaptoethanol.

In certain embodiments, the cell culture medium is an E8/E6 medium. E8/E6 medium is a feeder-free and xeno-free medium that supports the growth and expansion of human pluripotent stem cells. E8/E6 medium has been proven to support somatic cell reprogramming. In addition, E8/E6 medium can be used as a base for the formulation of custom media for the culture of PSCs. One example E8/E6 medium is described in Chen et al., Nat Methods 2011 May; 8(5):424-9, which is incorporated by reference in its entirety. One example E8/E6 medium is disclosed in WO15/077648, which is incorporated by reference in its entirety. In certain embodiments, an E8/E6 cell culture medium comprises DMEM/F12, ascorbic acid, selenium, insulin, NaHCO₃, transferrin, FGF2 and TGFβ. The E8/E6 medium differs from a KSR medium in that E8/E6 medium does not include an active BMP ingredient. Thus, in certain embodiments, when an E8/E6 medium is used to culture the presently disclosed stem cells to differentiate into sacral neural crest lineage cells, at least one BMP inhibitor is not required to be added to the E8/E6 medium. In certain embodiments, the when an E8/E6 medium is used to culture the presently disclosed stem cells to differentiate into sacral neural crest lineage cells, at least one BMP is added to the E8/E6 medium.

### 5.3. Cell Populations and Compositions

The presently disclosure provides a cell population of in vitro differentiated cells obtained by the methods disclosed herein, for example, in Section 5.2.

In certain embodiments, the presently disclosure provides a cell population of in vitro differentiated cells, wherein at least about 10%, at least about 20%, at least about 30%, at least about 35%, at least about 40%, at least about 50%, at least about 60%, or at least about 70% of the differentiated cells express at least one marker indicating a sacral neural crest lineage. Non-limiting examples of markers indicating a sacral neural crest lineage include Hox10 (including HoxA10, HoxB10, HoxC10, and HoxD10), Hox11 (including HoxA11, HoxB11, HoxC11, and HoxD11), Hox12 (including HoxA12, HoxB12, HoxC12, and HoxD12), or Hox13 (including HoxA13, HoxB13, HoxC13, and HoxD13), and combinations thereof. In certain embodiments, the presently disclosure provides a cell population of in vitro differentiated cells, wherein at least about 30%, at least about 35%, at least about 40%, at least about 50%, at least about 60%, or at least about 70% of the differentiated cells express at least one marker selected from Hox10 (including HoxA10, HoxB10, HoxC10, and HoxC10), Hox11 (including HoxA11, HoxB11, HoxC11, and HoxD11), Hox12 (including HoxA12, HoxB12, HoxC12, and HoxD12), and Hox13 (including HoxA13, HoxB13, HoxC13, and HoxD13), and combinations thereof. In certain embodiments, the in vitro differentiated cells are obtained by the differentiation methods described herewith, for example, in Section 5.2.

In certain embodiments, the differentiated sacral neural crest lineage cells further express at least one general neural crest marker. Non-limiting examples of general neural crest marker include forkhead box D3 (FOXD3), transcription factor AP-2 alpha (TFAP2A), T-box 2 (TBX2), RP4-792G4.2, RNA, 28S ribosomal 5 (RNA28S5), transcription factor AP-2 beta (TFAP2B), inscrutable homolog (INSC), RP11-200A13.2, cilia and flagella associated protein 126 (C1orf192), retinoid X receptor gamma (RXRG), complement factor H (CFH), and SOX10.

In certain embodiments, the differentiated sacral neural crest lineage cells further express at least one SOX10 neural crest lineage marker. In certain embodiments, the SOX10⁺ neural crest lineage marker is CD49D, P75NTR, and HNK1. [

In certain embodiments, the presently disclosure provides a cell population of in vitro differentiated cells, wherein at least about 10%, at least about 20%, at least about 30%, at least about 40%, at least about 50%, at least about 60%, at least about 70%, at least about 80%, or at least about 90% of the differentiated cells express at least one enteric neuron marker. Non-limiting examples of enteric neuron marker is selected from the group consisting of Tuj1, MAP2, PHOX2A, PHOX2B, TRKC, ASCL1, HAND2, EDNRB, 5HT, GABA, NOS, SST, TH, CHAT, DBH, Substance P, VIP, NPY, GnRH, CORP, and combinations thereof. In certain embodiments, the in vitro differentiated cells are obtained by the differentiation methods described herewith, for example, in Section 5.2.

In certain embodiments, the presently disclosure provides a cell population of in vitro differentiated cells, wherein at least about 10%, at least about 20%, at least about 30%, at least about 40%, at least about 50%, at least about 60%, at least about 70%, at least about 80%, or at least about 90% of the differentiated cells express at least one enteric glia cell marker. Non-limiting examples of enteric glia cell marker is selected from the group consisting of GFAP, S100b, vimentin, conexin-43, SOX10, and combinations thereof. In certain embodiments, the in vitro differentiated cells are obtained by the differentiation methods described herewith, for example, in Section 5.2.

In certain embodiments, less than about 30% (e.g., less than about 25%, less than about 20%, less than about 15%, less than about 10%, less than about 5%, less than about 4%, less than about 3%, less than about 2%, less than about 1%, less than about 0.5%, or less than about 0.1%) of the presently disclosed population of cells do not express Hox10 (including HoxA10, HoxB10, HoxC10, and HoxD10), Hox11 (including HoxA11, HoxB11, HoxC11, and HoxD11), Hox12 (including HoxA12, HoxB12, HoxC12, and HoxD12), or Hox13 (including HoxA13, HoxB13, HoxC13, and HoxD13).

In certain embodiments, less than about 15% (e.g., less than about 10%, less than about 5%, less than about 4%, less than about 3%, less than about 2%, less than about 1%, less than about 0.5%, or less than about 0.1%) of the presently disclosed population of cells express one or more marker selected from the group consisting of stem cell markers, CNS markers.

Non-limiting examples of pluripotent stem cell markers include OCT4, NANOG, SOX2, LIN28, SSEA4 and SSEA3.

Non-limiting examples of CNS markers include PAX6, NESTIN, FOXG1, SOX2, TBR1, TBR2 and SOX1.

In addition, the present disclosure provides compositions comprising any of the cell populations disclosed herein.

In certain embodiments, the cells are comprised in a composition that further comprises a biocompatible scaffold or matrix, for example, a biocompatible three-dimensional scaffold that facilitates tissue regeneration when the cells are implanted or grafted to a subject. In certain embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties). In certain embodiments, the composition further comprises growth factors for promoting maturation of the implanted/grafted cells into enteric neurons.

In certain embodiments, the composition comprises a cell population of from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed sacral neural crest lineage cells or enteric neurons.

In certain embodiments, said composition is frozen. In certain embodiments, said composition further comprises at least one cryoprotectant, for example, but not limited to, dimethylsulfoxide (DMSO), glycerol, polyethylene glycol, sucrose, trehalose, dextrose, or a combination thereof.

In certain embodiments, the composition is a pharmaceutical composition that comprises a pharmaceutically acceptable carrier, excipient, diluent or a combination thereof. The compositions can be used for preventing and/or treating enteric neuron related disorders, e.g., Hirschsprung disease (HD).

The present disclosure also provides a device comprising the differentiated cells or the composition comprising thereof, as disclosed herein. Non-limiting examples of devices include syringes, fine glass tubes, stereotactic needles and cannulas.

### 5.4. Methods of Preventing, Modeling and/or Treating Enteric Nervous System Disorders

In certain embodiments, the present disclosure provides methods for preventing, modeling, and/or treating an enteric nervous system (ENS) disorder using the cell populations and compositions disclosed herein (e.g., those disclosed in Section 5.3).

In certain embodiments, the methods comprise administering the presently disclosed stem-cell-derived sacral neural crest lineage cells, enteric neurons derived from the presently disclosed sacral neural crest lineage cells (e.g., an effective amount of the sacral neural crest lineage cells or the enteric neurons), or a composition comprising thereof into a subject suffering from an enteric nervous system disorder. In certain embodiments, the composition is a pharmaceutical composition which further comprises a pharmaceutically acceptable carrier.

Non-limiting examples of ENS disorders include Hirschsprung's disease (HD), toxic megacolon, any intestinal aganglionosis, irritable bowel syndrome, inflammatory bowel disease, gastroparesis, bowel-related drug side effects or other treatment complications. In certain embodiments, the ENS disorder is Hirschsprung's disease (HD).

The presently disclosed sacral neural crest lineage cells and/or enteric neurons can be administered or provided systemically or directly to a subject for treating or preventing an ENS disorder. In certain embodiments, the presently disclosed sacral neural crest lineage cells and/or enteric neurons are directly injected into an organ of interest (e.g., an organ affected by an ENS disorder (e.g., HD)). The presently disclosed sacral neural crest lineage cells and/or enteric neurons can be administered (injected) directly to a subject's intestine region, e.g., small intestine, colon, cecum, and/or rectum the. In certain embodiments, the presently disclosed sacral neural crest lineage cells and/or enteric neurons are administered to the cecum of a subject suffering from an ENS disorder (e.g., HD). In addition, the presently disclosed sacral neural crest lineage cells and/or enteric neurons can be administered (injected) directly to the wall, smooth muscle, connective tissues and/or lymphatic ducts of small intestine, colon, cecum, and/or rectum. In certain embodiments, the presently disclosed sacral neural crest lineage cells and/or enteric neurons are administered to the wall of the cecum of a subject suffering from an ENS disorder (e.g., HD). The injected cells can migrate to the smooth muscle of small intestine, colon, cecum and/or rectum, and form functional neuromuscular junction.

The presently disclosed sacral neural crest lineage cells and/or enteric neurons can be administered in any physiologically acceptable vehicle. Pharmaceutical compositions comprising the presently disclosed sacral neural crest lineage cells and/or enteric neurons and a pharmaceutically acceptable carrier are also provided. The presently disclosed sacral neural crest lineage cells and/or enteric neurons and the pharmaceutical compositions comprising thereof can be administered via localized injection, orthotropic (OT) injection, systemic injection, intravenous injection, or parenteral administration. In certain embodiments, the presently disclosed sacral neural crest lineage cells and/or enteric neurons are administered to a subject suffering from an ENS disorder (e.g., HD) via orthotropic (OT) injection.

The presently disclosed sacral neural crest lineage cells and/or enteric neurons and the pharmaceutical compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the present disclosure, e.g., a composition comprising the presently disclosed sacral neural crest lineage cells and/or enteric neurons, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the present disclosure, however, any vehicle, diluent, or additive used would have to be compatible with the presently disclosed sacral neural crest lineage cells and/or enteric neurons.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed sacral neural crest lineage cells and/or enteric neurons. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the presently disclosed sacral neural crest lineage cells and/or enteric neurons is the quantity of cells necessary to achieve an optimal effect. An optimal effect include, but are not limited to, repopulation of gut, repopulation of colon, and repopulation of gut and colon of a subject suffering from an ENS disorder (e.g., HD), and/or improved function of the subject's intestine.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the ENS disorder (e.g., HD), or otherwise reduce the pathological consequences of the ENS disorder (e.g., HD). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount is an amount that is sufficient to repopulate gut, repopulate colon, or repopulate gut and colon of a subject suffering from an ENS disorder (e.g., HD). In certain embodiments, an effective amount is an amount that is sufficient to improve the function of the intestine of a subject suffering from an ENS disorder (e.g., HD), e.g., the improved function can be about 1%, about 5%, about 10%, about 20%, about 30%, about 40%, about 50%, about 60a, about 70%, about 80%, about 90%, about 95%, about 98%, about 99% or about 100% of the function of a normal person's intestine.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the presently disclosed sacral neural crest lineage cells and/or enteric neurons are administered to a subject. In certain embodiments, from about 1×10⁵ to about 1×10⁷ the presently disclosed sacral neural crest lineage cells and/or enteric neurons are administered to a subject suffering from an ENS disorder (e.g., HD). In certain embodiments, about 2×10⁵ the presently disclosed sacral neural crest lineage cells and/or enteric neurons are administered to a subject suffering from an ENS disorder (e.g., HD). In certain embodiments, from about 1×10⁶ to about 1×10⁷ the presently disclosed sacral neural crest lineage cells and/or enteric neurons are administered to a subject suffering from an ENS disorder (e.g., HD). In certain embodiments, from about 2×10⁶ to about 4×10⁶ the presently disclosed sacral neural crest lineage cells and/or enteric neurons are administered to a subject suffering from an ENS disorder (e.g., HD). The precise determination of what would be considered an effective dose may be based on factors individual to each subject, including their size, age, sex, weight, and condition of the particular subject. Dosages can be readily ascertained by those skilled in the art from this disclosure and the knowledge in the art.

### 5.5 Kits

The present disclosure provides kits for inducing differentiation of stem cells to sacral neural crest lineage cells. In certain embodiments, the kits comprise (a) at least one activator of Wnt signaling, (b) at least one activator of FGF signaling, and (c) at least one molecule that induces sacral neural crest patterning. In certain embodiments, the kits further comprise (e) instructions for inducing differentiation of the stem cells into cells expressing at least one sacral neural crest lineage marker. In certain embodiments, the kits further comprise at least one BMP. In certain embodiments, the kits further comprise at least one inhibitor of SMAD signaling.

The present disclosure provides kits for inducing differentiation of stem cells to enteric neurons. In certain embodiments, the kits comprise (a) at least one activator of Wnt signaling; (b) at least one activator of FGF signaling; (c) at least one molecule that induces sacral neural crest patterning; (d) at least one growth factor; and (e) at least one Wnt activator. In certain embodiments, the kits comprise (f) instructions for inducing differentiation of the stem cells into cells expressing at least one enteric neuron marker or at least one enteric glia marker. In certain embodiments, the kits further comprise at least one BMP. In certain embodiments, the kits further comprise at least one inhibitor of SMAD signaling.

In certain embodiments, the at least one growth factor comprises FGF activators, glial cell line derived neurotrophic factor (GDNF), ascorbic acid, or a combination thereof.

In certain embodiments, the at least one molecule that induces sacral neural crest patterning is selected from the group consisting of GDF11, GDF8, and combinations thereof.

In certain embodiments, the instructions comprise contacting the stem cells with the inhibitor(s) and activator(s) in a specific sequence. The sequence of contacting the inhibitor(s) and activator(s) can be determined by the cell culture medium used for culturing the stem cells.

In certain embodiments, the instructions comprise contacting the stem cells with the inhibitor(s), activator(s), and molecule(s) as described by the methods of the present disclosure (see Section 5.2).

In certain embodiments, the present disclosure provides kits comprising an effective amount of a cell population or a composition disclosed herein in unit dosage form. In certain embodiments, the kits comprise a sterile container which contains the therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kits comprise instructions for administering the cell population or composition to a subject suffering from an ENS disorder. The instructions can comprise information about the use of the cells or composition for preventing, modeling, and/or treating an ENS disorder. In certain embodiments, the instructions comprise at least one of the following: description of the therapeutic agent; dosage schedule and administration for preventing, modeling, and/or treating at least a symptom in a subject having a neurological disorder or symptoms thereof; precautions; warnings; indications; counter-indications; over dosage information; adverse reactions; animal pharmacology; clinical studies; and/or references. The instructions can be printed directly on the container (when present), or as a label applied to the container, or as a separate sheet, pamphlet, card, or folder supplied in or with the container.

### 5.6. Exemplary Embodiments

A1. In certain non-limiting embodiments, the present disclosure provides an in vitro method for inducing differentiation of stem cells, comprising activation of wingless (Wnt) signaling, activation of fibroblast growth factor (FGF) signaling, and sacral neural crest patterning in the stem cells to obtain a population of differentiated cells expressing at least one marker indicating a sacral neural crest lineage.

A2. The foregoing method of A1, comprising contacting the stem cells with at least one activator of Wnt signaling, at least one activator of FGF signaling, and at least one molecule that induces sacral neural crest patterning.

A3. The foregoing method of A1 or A2, wherein the cells are contacted with the at least one molecule that induces sacral neural crest patterning for at least about 1 days, and/or the sacral neural crest patterning is induced for at least about 1 days.

A4. The foregoing method of any one of A1-A3, wherein the cells are contacted with the at least one molecule that induces sacral neural crest patterning for up to about 20 days, and/or the sacral neural crest patterning is induced for up to about 20 days.

A5. The foregoing method of any one of A1-A4, wherein the cells are contacted with the at least one molecule that induces sacral neural crest patterning for about 3 days, and/or the sacral neural crest patterning is induced for about 3 days.

A6. The foregoing method of any one of A1-A5, wherein the cells are contacted with the at least one activator of FGF signaling for at least about 1 days, and/or the activation of FGF signaling is induced for at least about 1 days.

A7. The foregoing method of any one of A1-A6, wherein the cells are contacted with the at least one activator of FGF signaling for up to about 8 days, and/or the activation of FGF signaling is induced for at least about 8 days.

A8. The foregoing method of any one of A1-A7, wherein the cells are contacted with the at least one activator of FGF signaling for about 3 days, and/or the activation of FGF signaling is induced for about 3 days.

A9. The foregoing method of any one of A1-A8, wherein the cells are contacted with the at least one activator of Wnt signaling for at least about 6 days, and/or the activation of Wnt signaling is induced for at least about 6 days.

A10. The foregoing method of any one of A1-A9, wherein the cells are contacted with the at least one activator of Wnt signaling for up to about 25 days, and/or the activation of Wnt signaling is induced for up to about 25 days.

A11. The foregoing method of any one of A1-A10, wherein the cells are contacted with at least one activator of Wnt signaling for about 20 days, and/or the activation of Wnt signaling is induced for about 20 days.

A12. The method of anyone of A1-A11, wherein the contact of the cells with the at least one molecule that induces sacral neural crest patterning is not initiated on the same day as the initial contact of the cell with the at least one activator of Wnt signaling, and/or the induction of sacral neural crest patterning is not initiated on the same day as the activation of Wnt signaling.

A13. The foregoing method of any one of A1-A12, wherein the contact of the cells with the at least one activator of FGF signaling is initiated on the same day as the initial contact of the cell with the at least one activator of Wnt signaling, and/or the induction of activation of FGF signaling is initiated on the same day as the activation of Wnt signaling.

A14. The foregoing method of any one of A1-A13, wherein the at least one molecule that induces sacral neural crest patterning is a member of transforming growth factor β (TGFβ) family, optionally wherein the at least one molecule that induces sacral neural crest patterning selected from the group consisting of GDF11, GDF8, and combinations thereof.

A15. The foregoing method of any one of A1-A14, wherein the at least one activator of FGF signaling is selected from the group consisting of FGF1, FGF2, FGF4, FGF6, FGF7, FGF8, FGF17, FGF18, and combination thereof.

A16. The foregoing method of any one of A1-A15, wherein the at least one activator of Wnt signaling activates canonical Wnt signaling.

A17. The foregoing method of any one of A1-A16, wherein the at least one activator of Wnt signaling comprises an inhibitor of glycogen synthase kinase 3β (GSK3β) signaling.

A18. The foregoing method of any one of A1-A17, wherein the at least one activator of Wnt signaling is selected from the group consisting of CHIR99021. CHIR98014. AMBMP hydrochloride, LP 922056, Lithium, BIO, SB-216763, Wnt3A, Wnt1, Wnt5a, derivatives thereof, and combinations thereof.

A19. The foregoing method of any one of A1-A18, wherein the at least one activator of Wnt signaling comprises CHIR99021.

A20. The foregoing method of any one of A1-A19, wherein the cells are further contacted with at least one inhibitor of Small Mothers Against Decapentaplegic (SMAD) signaling, and/or the method further comprises inducing inhibition of SMAD signaling

A21. The foregoing method of A20, wherein the cells are further contacted with the at least one inhibitor of SMAD signaling for at least about 1 day, and/or the inhibition of SMAD signaling is induced for at least about 1 day.

A22. The foregoing method of A20 or A21, wherein the cells are contacted with the at least one inhibitor of SMAD signaling for up to about 20 days, and/or the inhibition of SMAD signaling is induced for up to about 20 days.

A23. The foregoing method of anyone of A20-A22, wherein the cells are contacted with the at least one inhibitor of SMAD signaling for about 17 days, and/or the inhibition of SMAD signaling is induced for about 17 days.

A24. The foregoing method of any one of A20-A23, wherein the at least one inhibitor of SMAD signaling comprises an inhibitor of TGFβ/Activin-Nodal signaling, and/or the inhibition of SMAD signaling comprises inhibition of TGFβ/Activin-Nodal signaling.

A25. The foregoing method of A24, wherein the at least one inhibitor SMAD signaling further comprises an inhibitor of bone morphogenetic protein (BMP) signaling, and/or the inhibition of SMAD signaling further comprises inhibition of BMP signaling. A26. The foregoing method of A24, wherein the at least one inhibitor of TGFβ/Activin-Nodal signaling comprises an inhibitor of ALK5.

A27. The foregoing method of A24 or A26, wherein the at least one inhibitor of TGFβ/Activin-Nodal signaling is selected from the group consisting of SB431542, derivatives of SB431542, and combinations thereof.

A28. The foregoing method of A27, wherein the derivative of SB431542 comprises A83-01, and/or ResSox.

A29. The foregoing method of any one of A24, and A26-A28, wherein the at least one inhibitor of TGFβ/Activin-Nodal signaling comprises SB431542.

A30. The foregoing method of A25, wherein the at least one inhibitor of BMP signaling is selected from the group consisting of LDN193189. Noggin, dorsomorphin, derivatives of LDN193189, derivatives of Noggin, derivatives of dorsomorphin, and combinations thereof.

A31. The foregoing method of A25 or A30, wherein the at least one inhibitor of BMP comprises LDN-193189.

A32. The foregoing method of any one of A1-A31, wherein the cells are contacted with at least one bone morphogenetic protein (BMP), and/or the method further comprises inducing activation of BMP signaling.

A33. The foregoing method of A32, wherein the cells are contacted with the at least one BMP for at least about 1 days, and/or the activation of BMP signaling is induced for at least about 1 days.

A34. The foregoing method of A32 or A33, wherein the cells are contacted with the at least one BMP for up to about 25 days, and/or the activation of BMP signaling is induced for up to about 25 days.

A35. The foregoing method of any one of A32-A34, wherein the cells are contacted with at least one BMP for about 20 days, and/or the activation of BMP signaling is induced for about 20 days.

A36. The foregoing method of any one of A32-A35, wherein the at least one BMP is selected from the group consisting of BMP1, BMP2, BMP3, BMP4, BMP5, BMP6, BMP7, BMP8a, BMP8b, BMP10. BMP11, BMP15, and combinations thereof.

A37. The foregoing method of any one of A32-A36, wherein the at least one BMP comprises BMP2, BMP4, or a combination thereof.

A38. The foregoing method of any one of A1-A37, wherein at least about 70% of the cells express the at least one sacral neural crest lineage marker at least about 20 days from the initial contact of the stem cells with the at least one activator of Wnt signaling, and/or from the initiation of the induction of activation of Wnt signaling.

A39. The foregoing method of any one of A1-A38, wherein the at least one sacral neural crest lineage marker is selected from the group consisting of Hox10, Hox11, Hox12, and Hox13, and combinations thereof.

A40. The foregoing method of any one of A1-A39, wherein the differentiated cells further express at least one SOX10+ neural crest lineage marker.

A41. The foregoing method of A40, wherein the at least one SOX10+ neural crest lineage marker comprises CD49D.

A42. The foregoing method of anyone of A1-A41, wherein the stem cells are pluripotent stem cells.

A43. The foregoing method of any one of A1-A42, wherein the stem cells are human stem cells.

A44. The foregoing method of any one of A1-A43, wherein the stem cells are selected from the group consisting of embryonic stem cells, induced pluripotent stem cells, parthenogenetic stem cells, primordial germ cell-like pluripotent stem cells, epiblast stem cells, and F-class pluripotent stem cells, enhanced pluripotent stem cells, naive stage pluripotent stem cells, and combinations thereof.

A45. The foregoing method of any one of A1-A44, further comprising subject the differentiated cells to conditions favoring maturation of sacral neural crest lineage cells to cells that express at least one enteric neuron marker or at least one enteric glia cell marker.

A46. The foregoing method of A45, wherein the conditions comprise contacting the differentiated cells with at least one growth factor, at least one Wnt activator, or a combination thereof.

A47. The foregoing method of A46, wherein the at least one growth factor comprises at least one FGF activator, glial cell line derived neurotrophic factor (GDNF), ascorbic acid, or a combination thereof.

A48. The foregoing method of A47, wherein the differentiated cells are contacted with the at least one Wnt activator and the at least one FGF activator.

A49. The foregoing method of A48, wherein the differentiated cells are contacted with the at least one Wnt activator and the at least one FGF activator for about 4 days.

A50. The foregoing method of any one of A47-A49, wherein the differentiated cells are contacted with the at least one Wnt activator, the at least one FGF activator, GDNF, and ascorbic acid.

A51. The foregoing method of any one of A47-A50, wherein the at least one FC3F activator is selected from the group consisting of FGF2, FGF4, FGF7, and FGF8.

A52. The foregoing method of any one of A47-A50, wherein the at least one Wnt activator is selected from the group consisting of CHIR99021, CHIR98014, AMBMP hydrochloride, LP 922056, Lithium, BIO, SB-216763, Wnt3A, Wnt1, Wnt5a, derivatives thereof, and combinations thereof.

A53. The foregoing method of any one of A47-A52, wherein the at least one enteric neuron marker is selected from the group consisting of Tuj1, MAP2, PHOX2A, PHOX2B, TRKC, ASCL1, HAND2. EDNRB, SIT, GABA, NOS, SST, TH, CHAT, DBH, Substance P. VIP, NPY, GnRH, CGRP, and combinations thereof.

A54. The foregoing method of anyone of A47-A52, wherein the at least one enteric glia cell marker is selected from the group consisting of GFAP, S100b, vimentin, conexin-43, SOX10, and combinations thereof.

B1. A cell population of in vitro differentiated cells expressing at least one sacral neural crest lineage marker obtained by a method of any one of A1-A44.

C1. A cell population of in vitro differentiated cells expressing at least one enteric neuron marker obtained by a method of any one of A45-A54.

D1. A composition comprising the cell population of B1 OR C1.

D2. The foregoing composition of D1, which is a pharmaceutical composition further comprising a pharmaceutically acceptable carrier.

E1. A kit for inducing differentiation of stem cells, comprising:
(a) at least one activator of Wnt signaling;
(b) at least one activator of FGF signaling;
(c) at least one molecule that induces sacral neural crest patterning; and
(d) instructions for inducing differentiation of the stem cells into cells expressing at least one sacral neural crest lineage marker.

E2. The foregoing kit of E1, further comprising at least one inhibitor of SMAD signaling.

E3. The foregoing kit of E1 or E2, further comprising at least one BMP.

E4. The foregoing kit of any one of E1-E3, wherein the at least one molecule that induces sacral neural crest patterning is selected from the group consisting of GDF11, GDF8, and combinations thereof.

F1. A kit for inducing differentiation of stem cells, comprising:
(a) at least one activator of Wnt signaling;
(b) at least one activator of FGF signaling;
(c) at least one molecule that induces sacral neural crest patterning;
(d) at least one growth factor.
(e) at least one Wnt activator; and
(f) instructions for inducing differentiation of the stem cells into cells expressing at least one enteric neuron marker.

F2. The foregoing kit of F1, further comprising at least one inhibitor of SMAD signaling.

F3. The foregoing kit of F1 OR F2, further comprises at least one BMP.

F4. The foregoing kit of any one of E1-E4 or F1-F3, wherein the at least one growth factor comprises FGF activators, glial cell line derived neurotrophic factor (GDNF), ascorbic acid, or a combination thereof.

G1. A method of preventing and/or treating an enteric nervous system disorder in a subject in need thereof, comprising administering to the subject an effective amount of one of the followings:
(a) the cell population of B1 or C1; or
(b) the composition of D1 or D2.

G2. The foregoing method of G1, wherein the enteric nervous system disorder is Hirschsprung's disease.

G3. The foregoing cell population of B1 or CA or the foregoing composition of D1 or D2 for use in preventing and/or treating an enteric nervous system disorder in a subject in need thereof.

G4. The foregoing cell population or composition for use of G3, wherein the enteric nervous system disorder is Hirschsprung's disease.

### 6. Example

The present disclosure will be better understood by reference to the following Example, which is provided as exemplary of the present disclosure, and not by way of limitation.

### Example 1: Exemplary Protocol for Derivation of Sacral Neural Crest Lineage Cells from Human Pluripotent Stem Cells

This Example describes an exemplary protocol for producing sacral neural crest lineage cells. Before the induction of sacral NC cells, the hPSC cells were kept in a chemically defined and feeder free culture system named E8. The hPSCs were cultured in the 10-cm dishes, which were coated with Vitronectin. 10 ml of E8 media were added to each plate and the media were changed every day. When the cells reached around 70% confluency, EDTA was used to dissociate the cells and the cells were passed at a 1:15 ratio.

From Day 0 to Day 3, the cells were cultured in an induction medium A. The induction medium A was an E6 medium comprising 100 ng/ml of FGF2, 3 μM of CHIR99021, 1 ng/ml of BMP4, and 50 ng/ml of GDF11. The cells were fed every day with 2 ml induction medium per 24 well.

From Day 4 to Day 20, the cells were cultured in an induction medium B. The induction medium B was an E6 medium comprising 10 μM of SB431542, 1.5 μM of CHIR99021, and 1 ng/ml of BMP4. The cells were fed every other day with 2 ml induction medium per 24 well.

Details are shown in FIG. 1.

### Example 2: Generation of Sacral Neural Crest Lineage Cell from Stem Cells In Vitro

Human enteric neuron system (ENS) develops from two different types of neural crest cells (NCs), vagal neural crest lineage cells (VNCs) and sacral neural crest lineage cells (SNCs). VNCs are located at the anterior of the body. During development, VNCs invade the gut from the anal part and migrate down to cover the whole gut. SNCs are located at the most posterior part of the body axis. During development, SNCs invade the gut from the distal part and migrate up to meet the VNCs. One difference between VNC and SNC is their anterior-posterior (AP) identity, which is characterized by the expression of different Hox genes. For example, cranial neural crest cells (CNCs) are located at the most anterior part and do not express Hox genes. VNCs express Hox genes from Hox2 to Hox5. SNCs, as the most posterior neural crest cells, express Hox genes from Hox2 to Hox13. It has been shown that VNCs can be generated and differentiated from human stem cells and that VNCs can be matured into ENS lineages in vitro (Fattahi et al., Nature. 2016 Mar. 3; 531(7592):105-9)

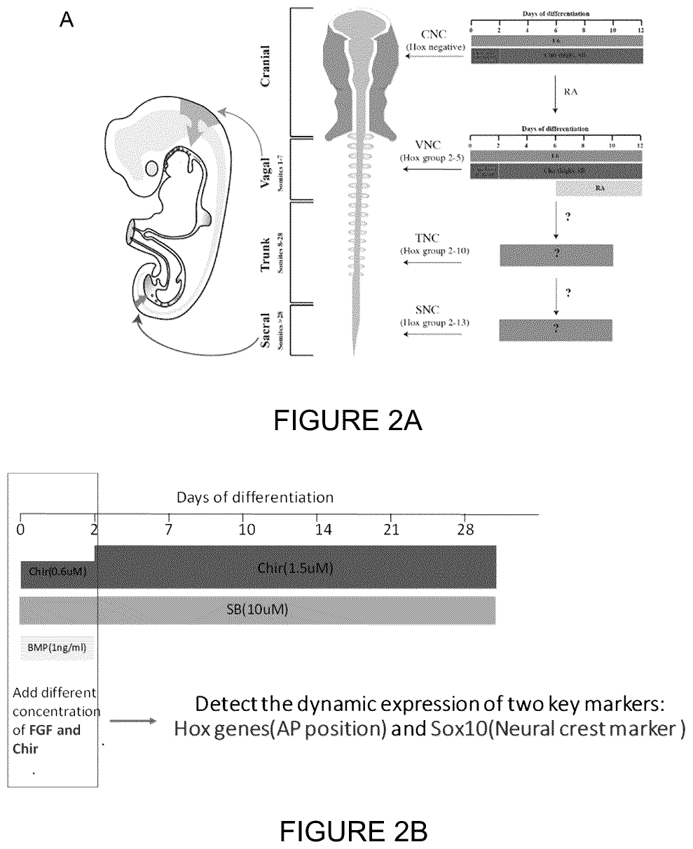

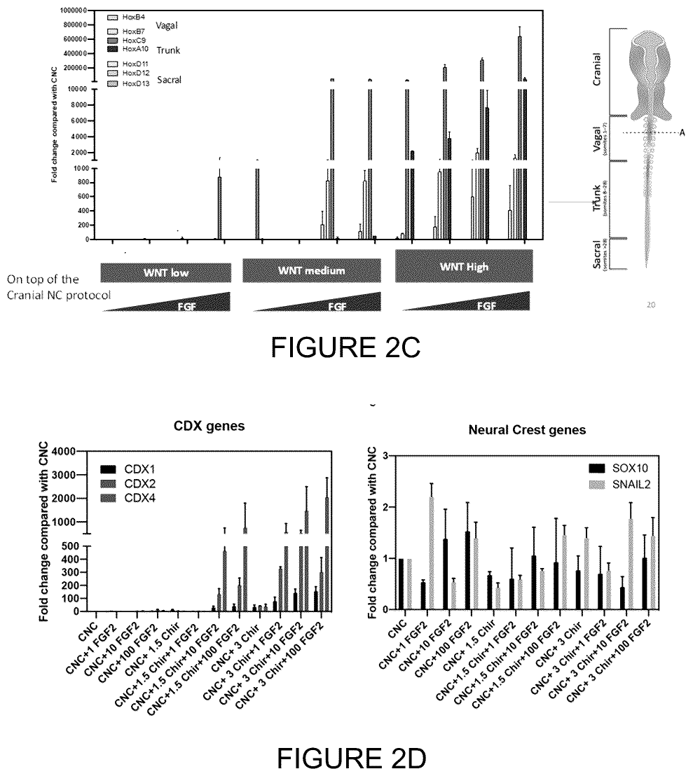

FIG. 2A demonstrates an exemplary protocol for differentiating stem cells into cranial neural crest cells (CNCs) or VNCs.

To generate SNCs in vitro, different concentrations of FGF2 and CHIR99021 (an activator of Wnt signaling) were tested, and the resulting Hox gene expressions were determined (FIG. 2B). As shown in FIGS. 1C and 1D, activation of FGF and Wnt signals induced the expression of posterior genes HoxB7, HoxC9, HoxA10, and CDX4. Thus, high concentrations of FGF2 and CHIR99021 helped patterning the cells to trunk neural crest cells (TNCs). However, additional factors were needed for the transition from TNCs to SNCs.

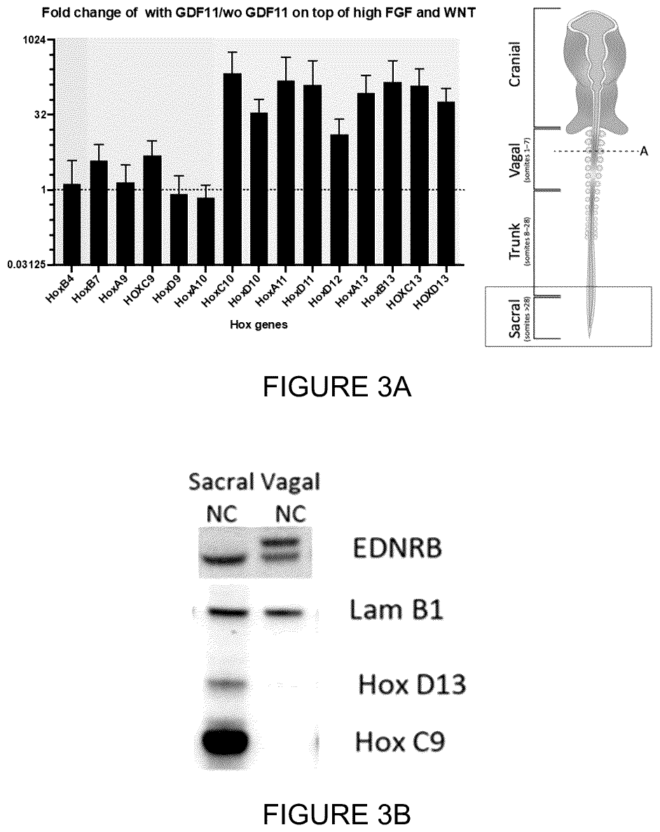

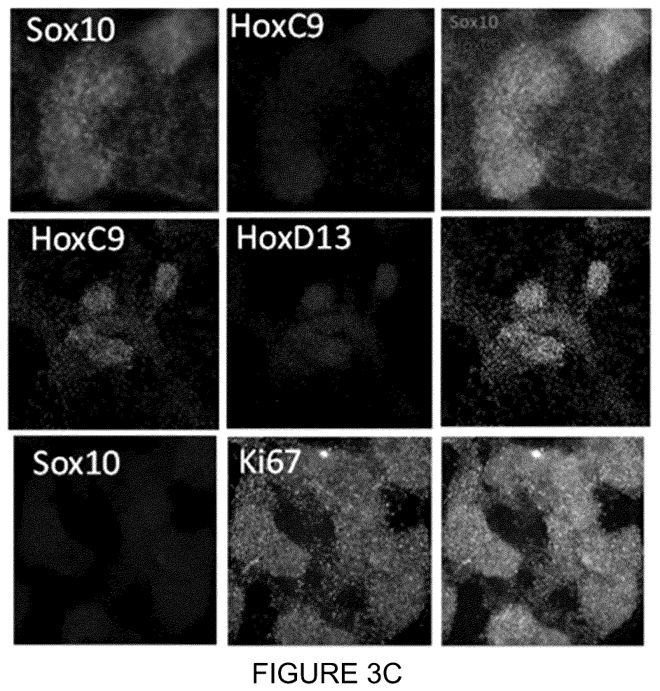

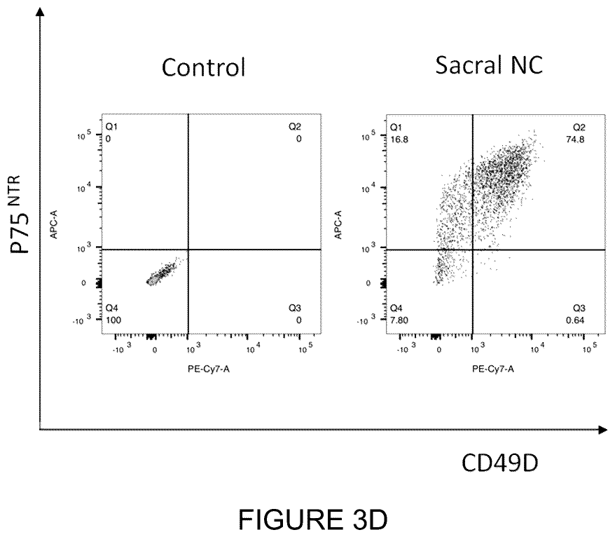

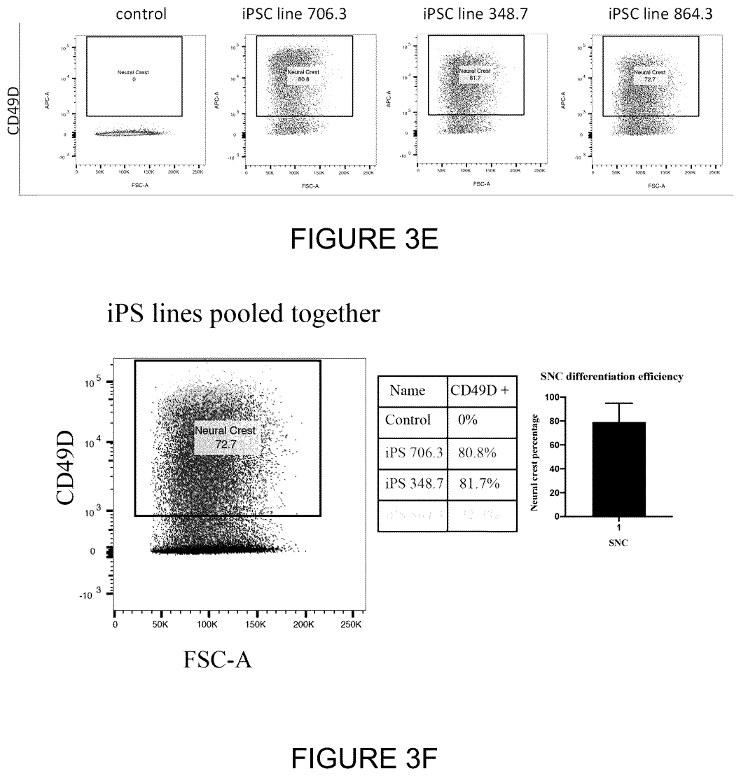

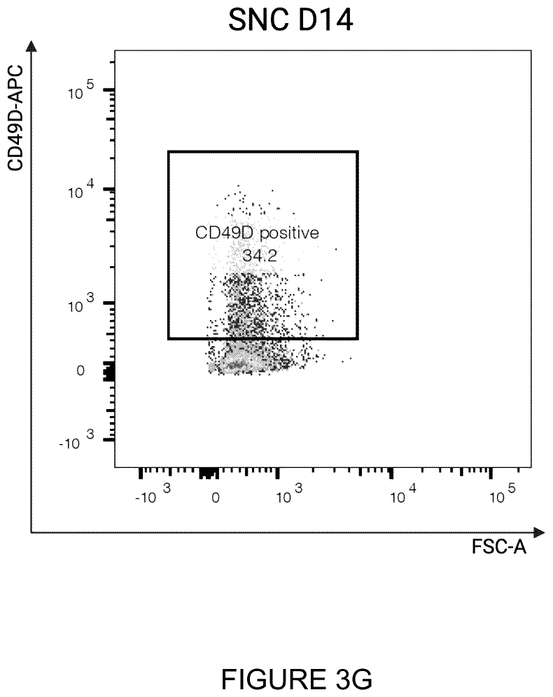

The present disclosure discovered that GDF11 promoted the transition from TNCs to SNCs (FIG. 3A). The SNCs were characterized by detecting the expression of HoxD13 and HoxC9 as AP identity (FIG. 3B), Sox10 as neural crest marker (FIG. 3C), and CD49D⁺ and P75NTR as cell surface markers (FIG. 3D). The present disclosure further demonstrated that the SNC differentiation protocol worked with different iPSC lines (FIGS. 3E and 3F). FIG. 3G shows that 34.2% cells were CD49D⁺ sacral neural crest at day 14 of differentiation.

Therefore, FGF2 and CHIR99021 promoted the patterning of most caudal Hox codes, while GDF11 further patterned NC to sacral level. The present disclosure discovered that SNCs can be generated by day 20 of differentiation. Moreover, the temporal collinearity expression of Hox genes in vertebrate AP axial pattern can be reproduced in vitro.

### Example 3: Dissection of the SNC Differentiation Protocol

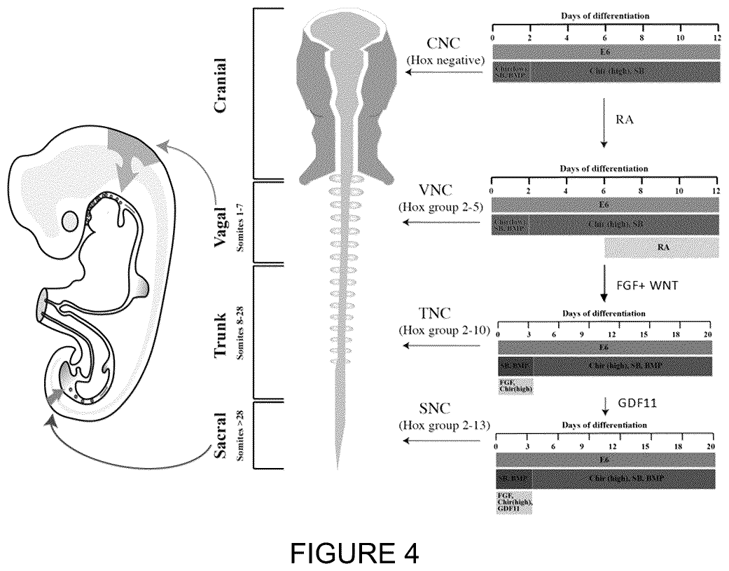

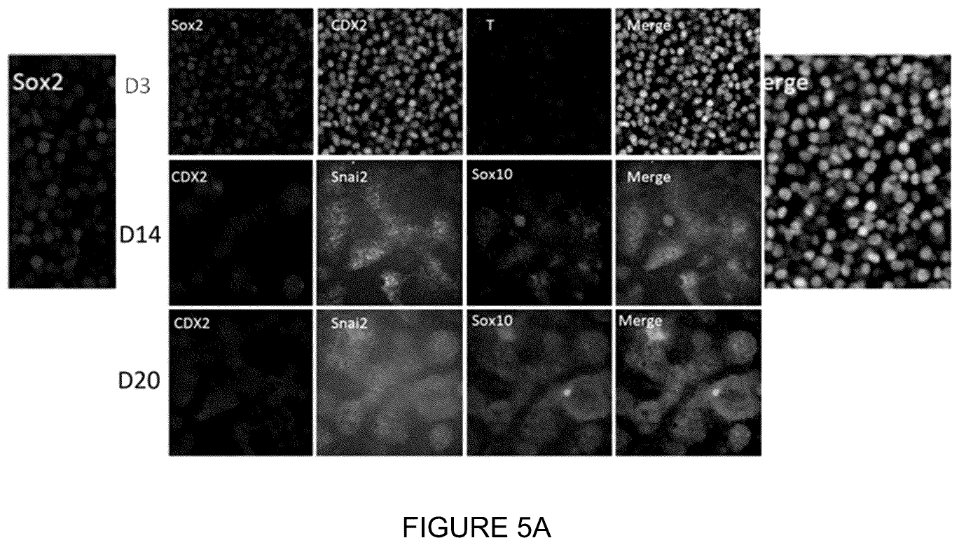

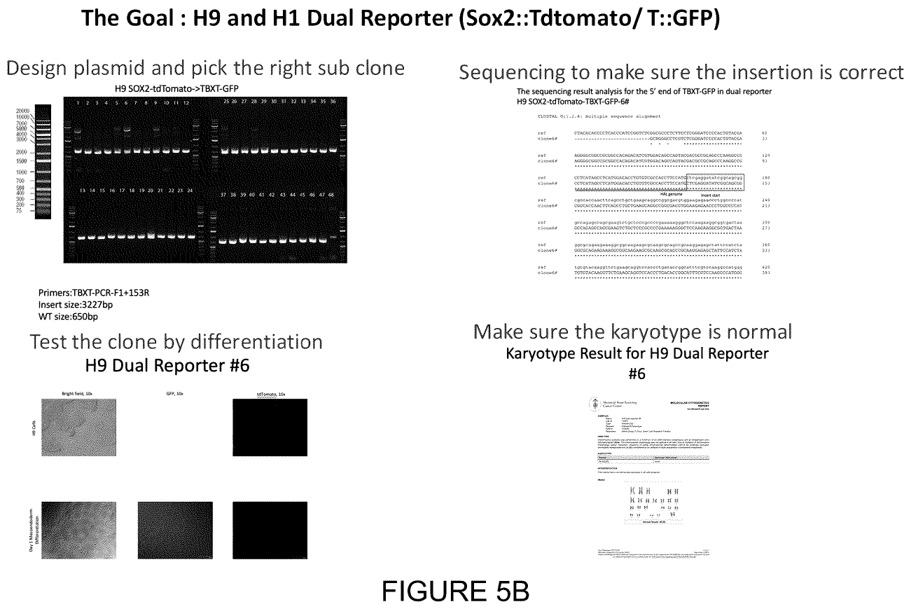

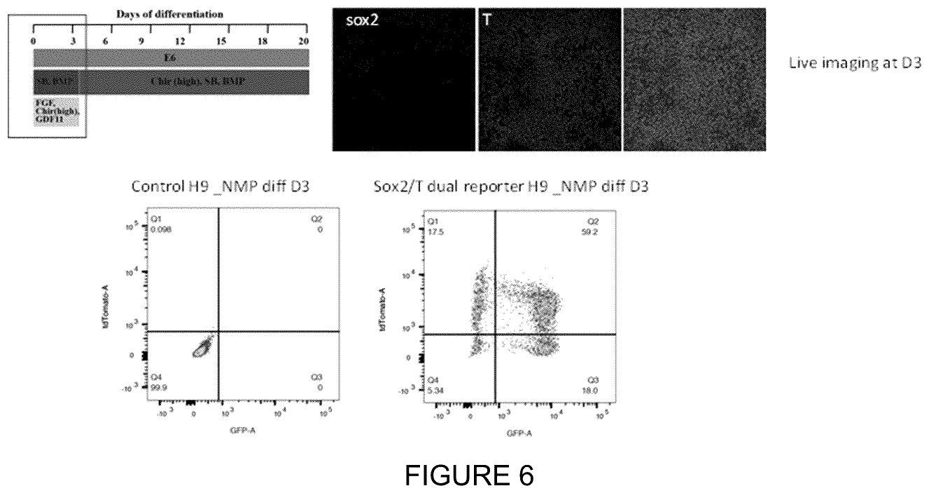

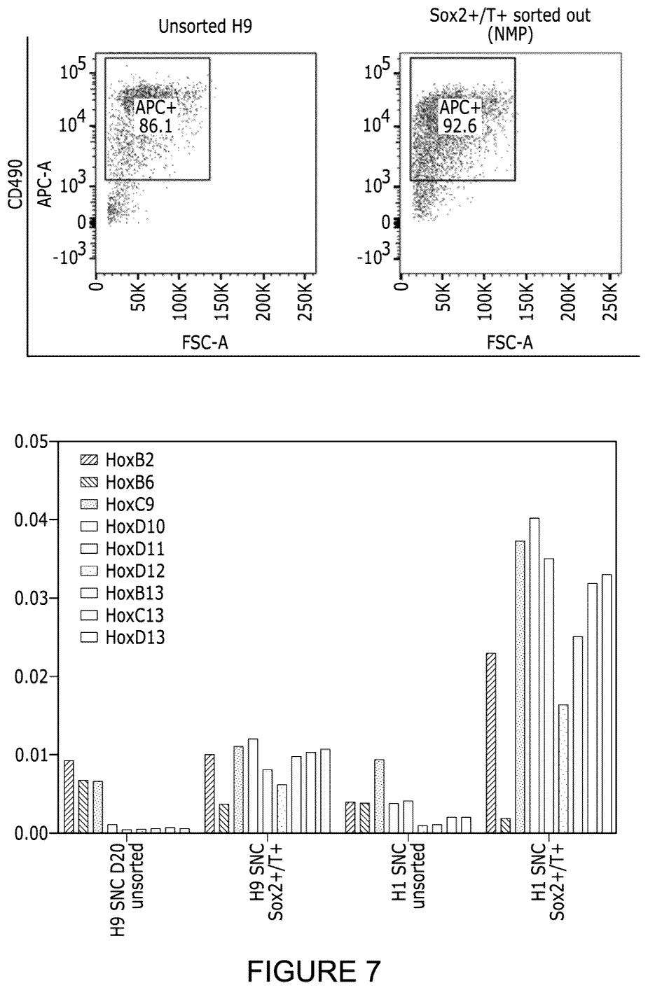

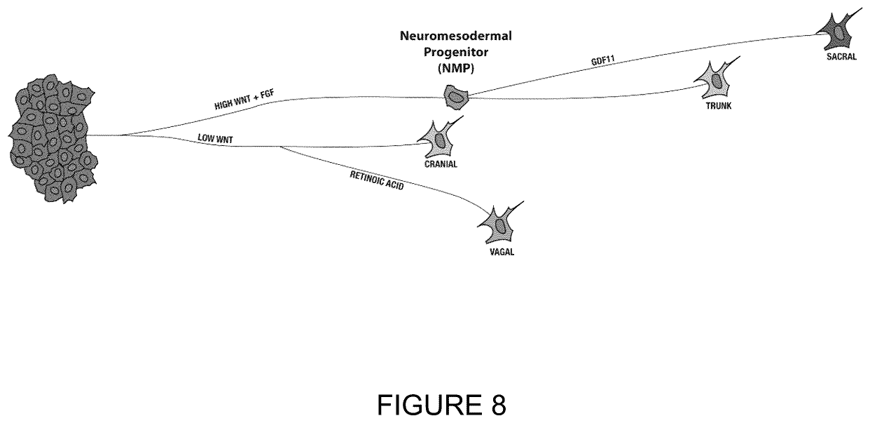

The present disclosure further tested whether the VNCs and SNCs came from the same progenitors (FIG. 4). Neuromesodermal progenitors (NMPs), newly identified posterior progenitors, were characterized in FIG. 5A. New NMP reporter cell lines were generated (FIG. 5B) and differentiated into SNCs using the presently disclosed methods (FIG. 6). NMP reporter cell lines were sorted and differentiated into posterior NC in vitro (FIG. 7). The present disclosure discovered that SNCs came from NMPs (FIG. 8).

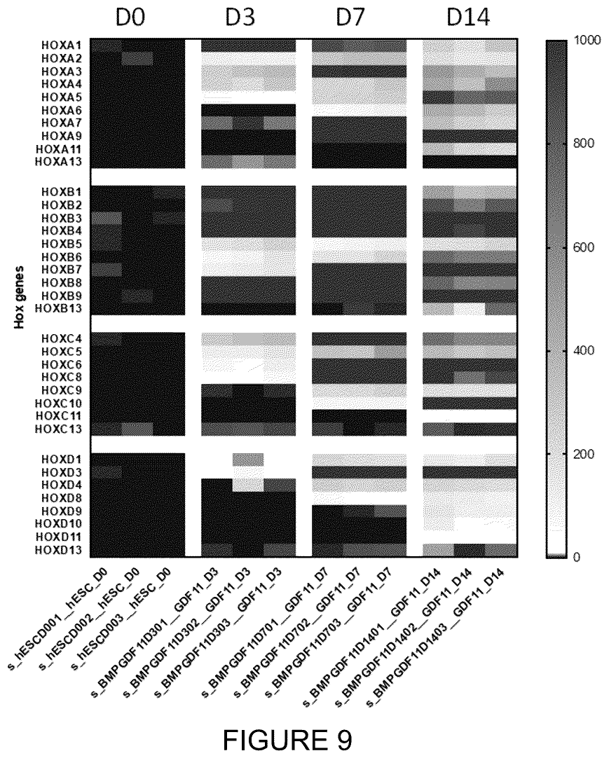

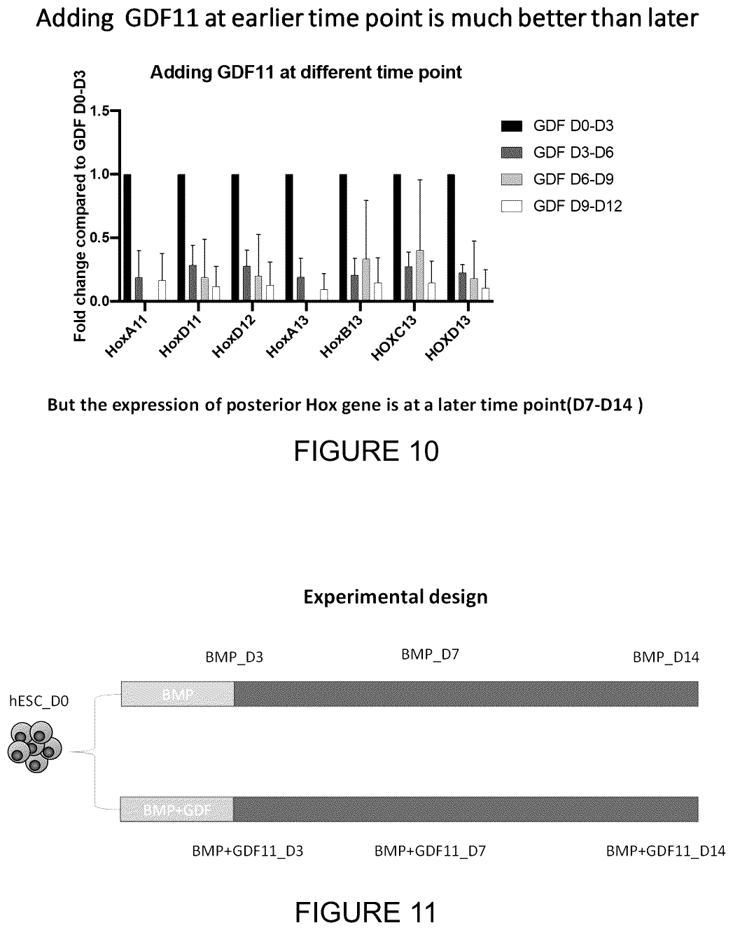

To understand the mechanism of GDF11 on the regulation of posterior Hox genes, cells from differentiation day 3 were collected. As shown in FIG. 9, early treatment with GDF11 induced posterior Hox gene expression at a later time point. Next, the present disclosure tested whether adding GDF11 at different time points during differentiation can induce the expression of Hox genes differently. The addition of GDF11 at earlier time points (from day 0 to day 3) during differentiation induced higher expression of posterior Hox genes than later time points (FIG. 10).

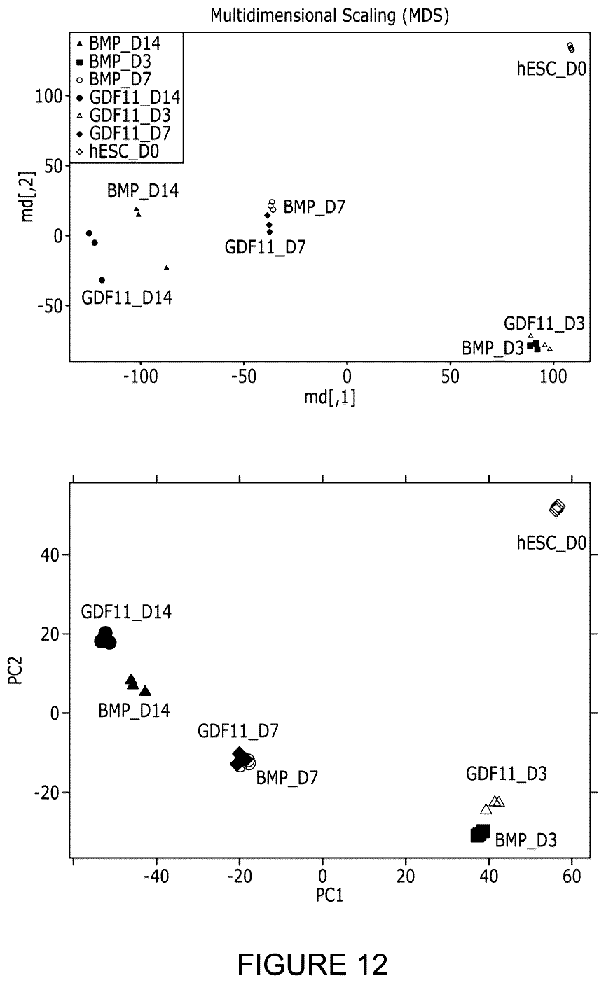

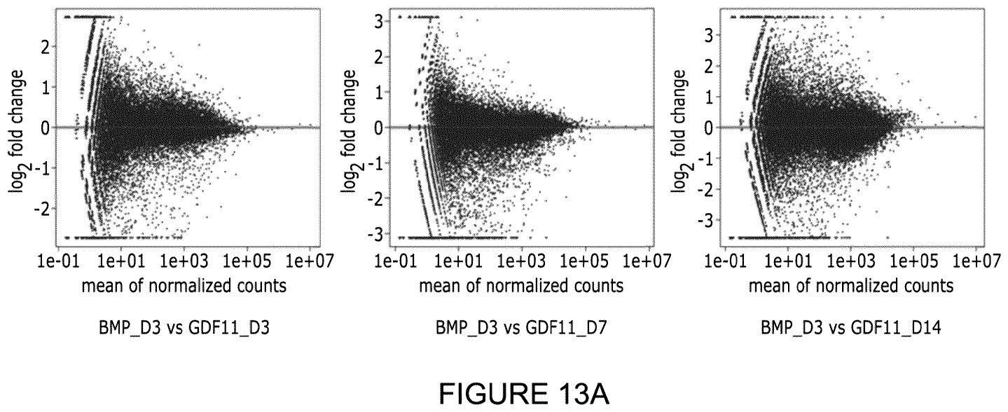

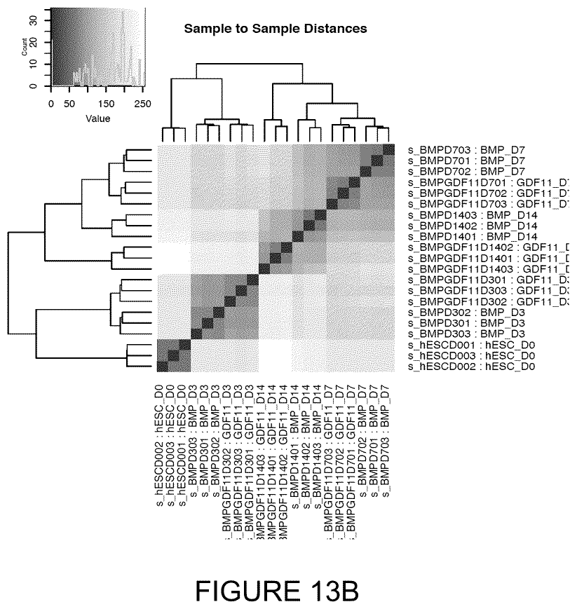

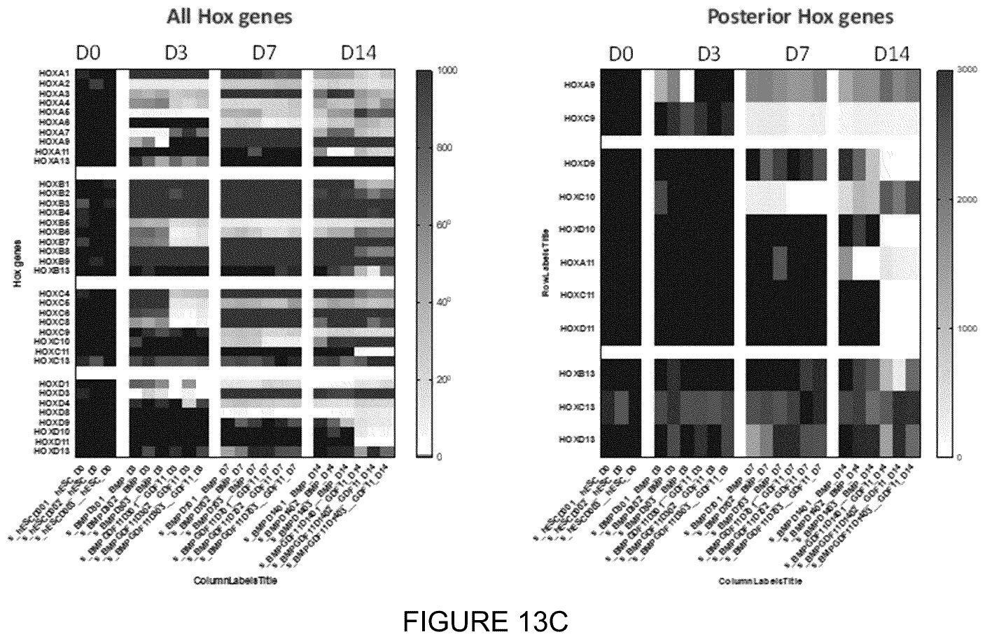

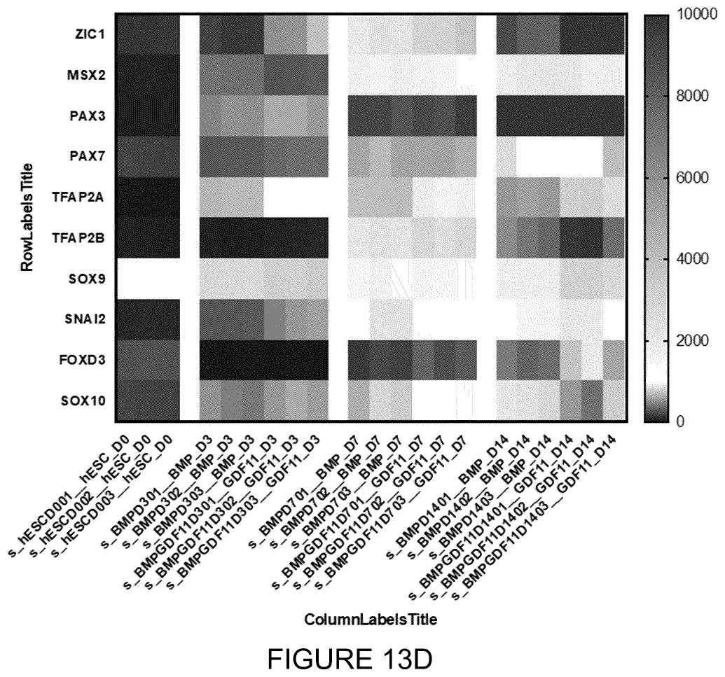

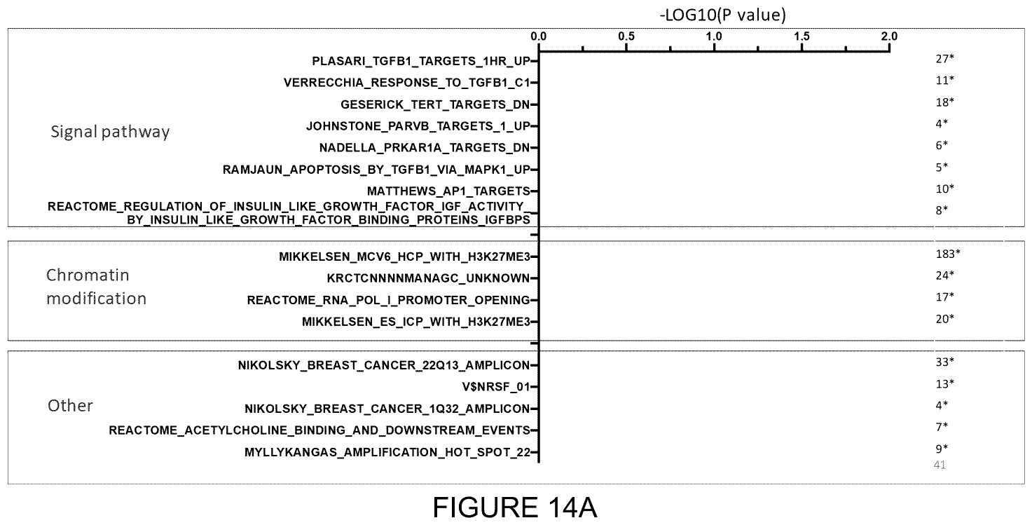

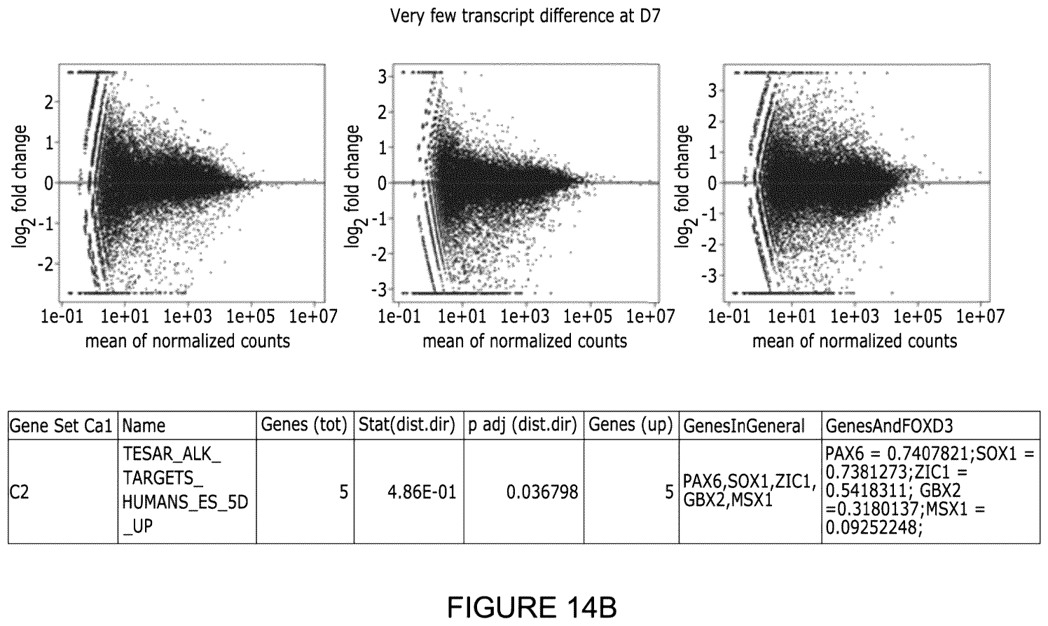

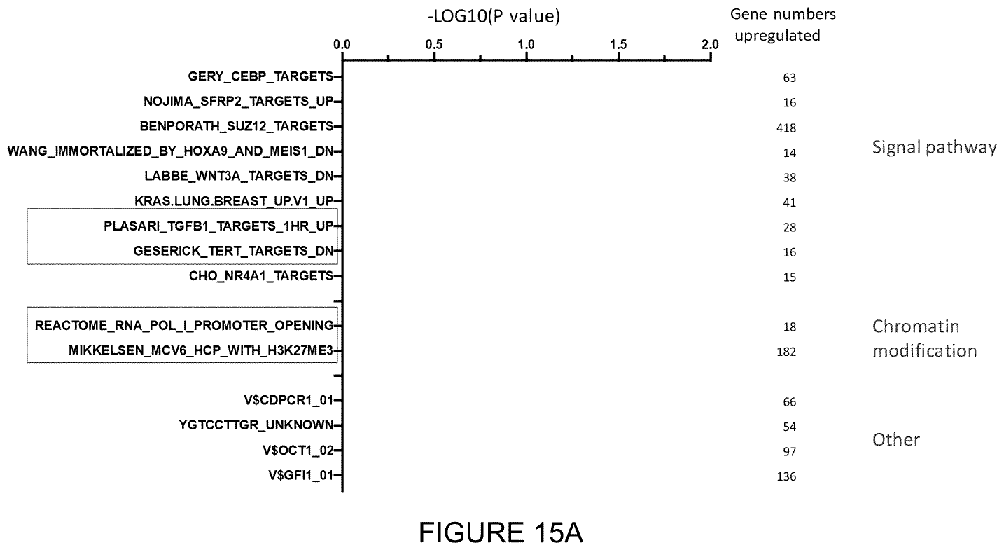

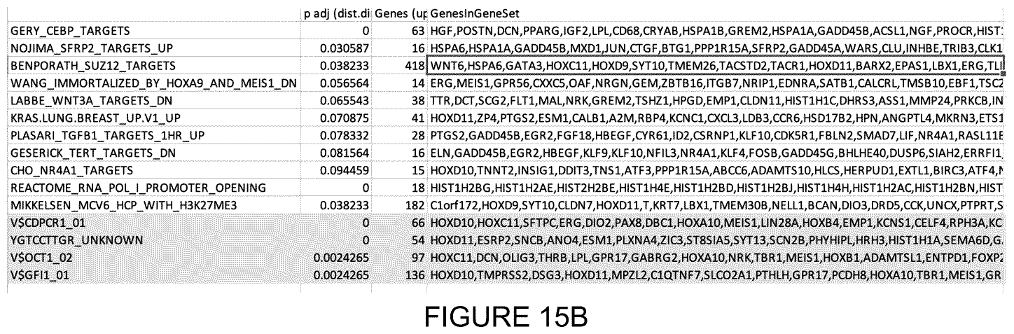

The present disclosure further discovered that early treatment of GDF11 primed the Hox genes expression through chromatin modification. Cells were collected on day 0, day 3, day 7 and day 14 of differentiation with or without GDF11 treatment (FIG. 11). ATAC seq and RNA seq were performed to examine the mechanism of GDF11 (FIGS. 12, 13A and 13B). Early treatment with GDF11 induced posterior Hox genes expression at later time points (FIG. 13C). Sorted NMPs generated posterior NC in vitro (FIG. 13D). Further, to provide an insight into the potential functional implications of the observed differences in chromatin accessibility and gene expression, gene sets enriched in GDF11 conditions were analyzed (FIGS. 14A, 14B, 15A, and 15B).

Therefore, the present disclosure discovered that NMPs were new posterior progenitors giving rise to the posterior neural crest cells. Moreover, early treatment with GDF11 induced posterior Hox genes expression at a later time point.

### Example 4: Comparison of the VNCs and SNCs In Vitro

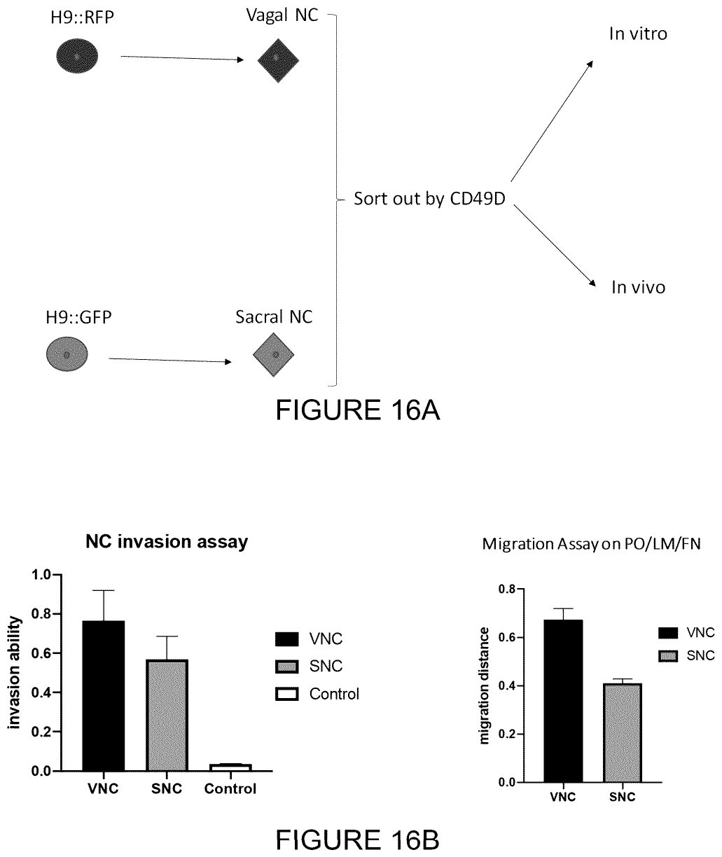

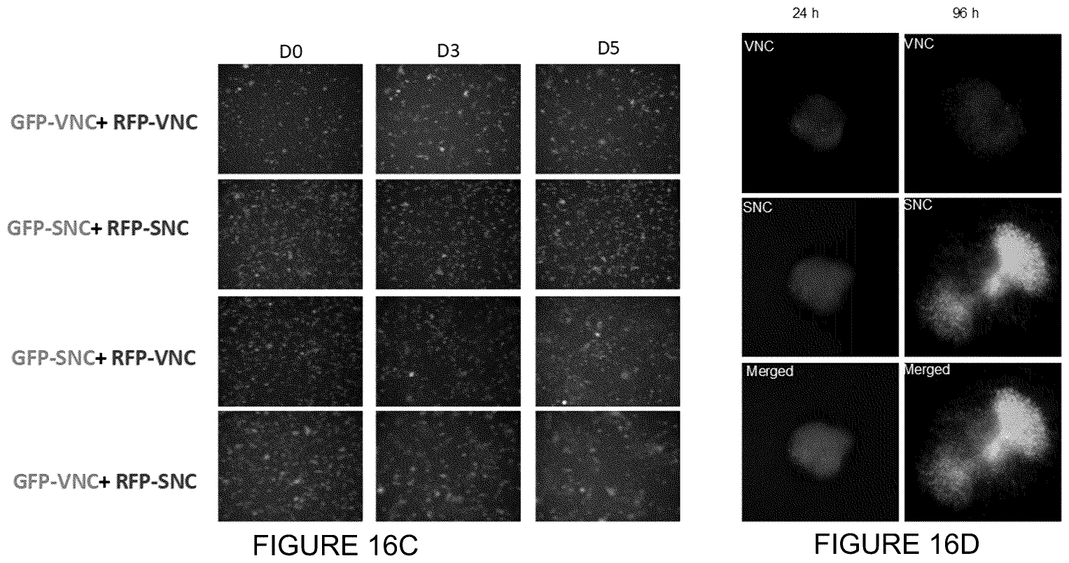

Differences between VNCs and SNCs were analyzed in vitro. Using different fluorescence-labeled cells, the migration and invasion abilities of VNCs and SNCs were determined (FIG. 16A). VNCs had stronger migration and invasion abilities than SNCs (FIG. 16B). Further, live imaging analysis showed that VNCs and SNCs had a repelling effect on each other (FIGS. 16C and 16D).

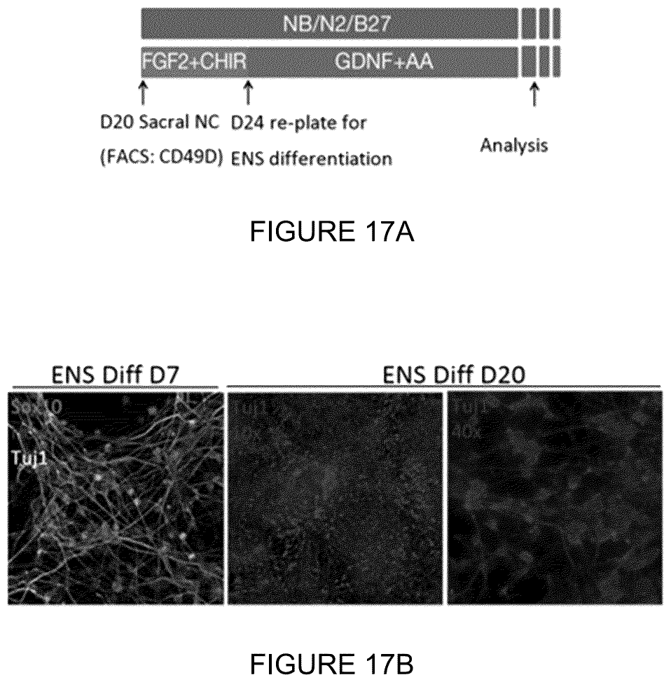

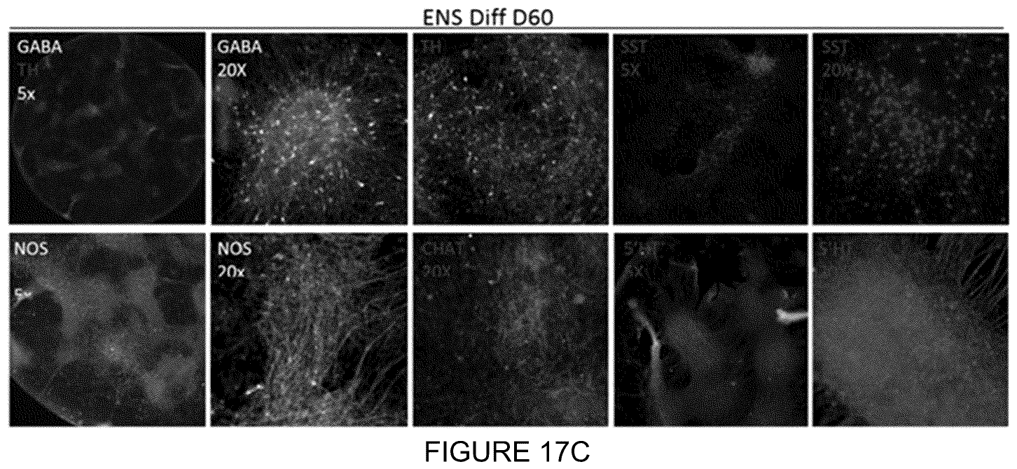

SNCs were further differentiated into enteric neurons according to the protocol depicted in FIG. 17A. Notably, SNCs differentiated into many subtypes of enteric neurons (FIGS. 17B and 17C).

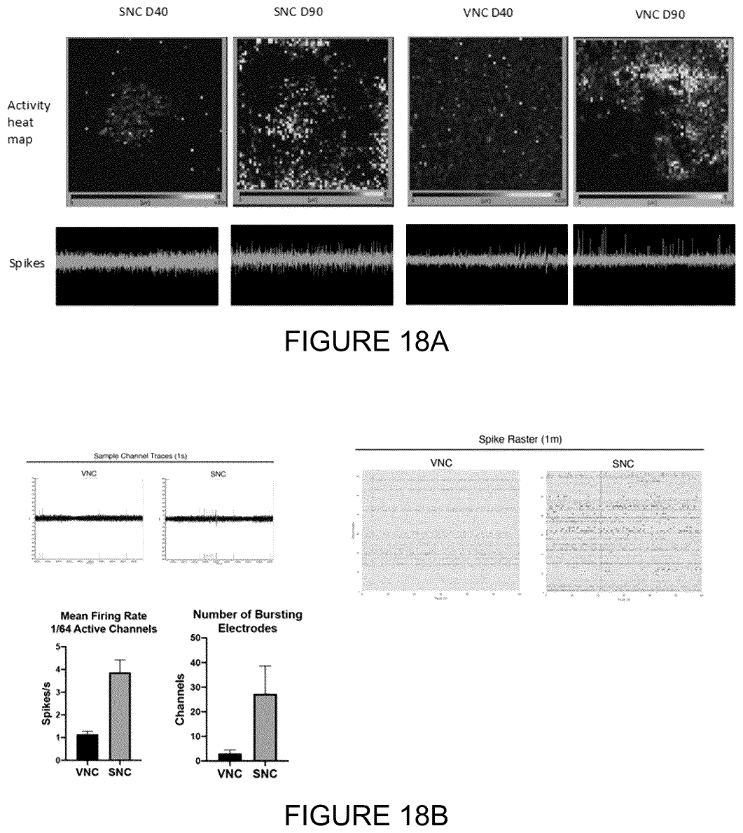

The electrophysiological activity of VNCs and SNCs was analyzed using a multielectrode array (MEA) (FIG. 18A). Briefly, SNCs or VNCs were plated on PO/LM/FN plates and differentiated into enteric neurons. On day 15 of the differentiation process, the neurons were dissociated into single cells and plated onto the chips at a density of 250,000 cell/cm². As shown in FIG. 18B, SNCs showed a higher neuronal activity as compared to the VNCs. SNC also showed an elevated firing rate and an increased number of bursting electrodes (FIG. 18C).

Overall, SNC and VNC showed different migration and invasion activities and repelled each other during migration. Further, the present disclosure showed that the SNCs can differentiate into different subtypes of enteric neurons and had higher neuronal activity as compared to the VNC in vitro.

### Example 5: Comparison of the VNCs and SNCs In Vivo

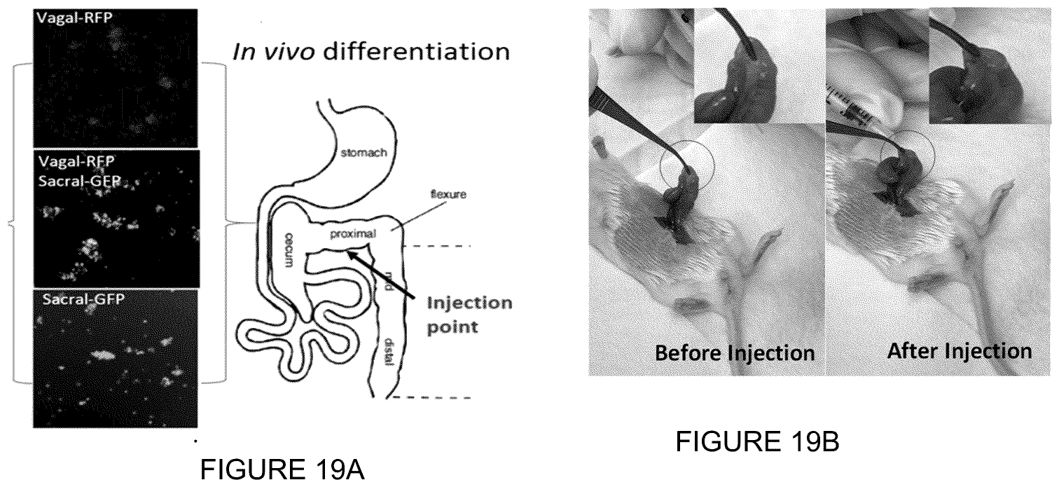

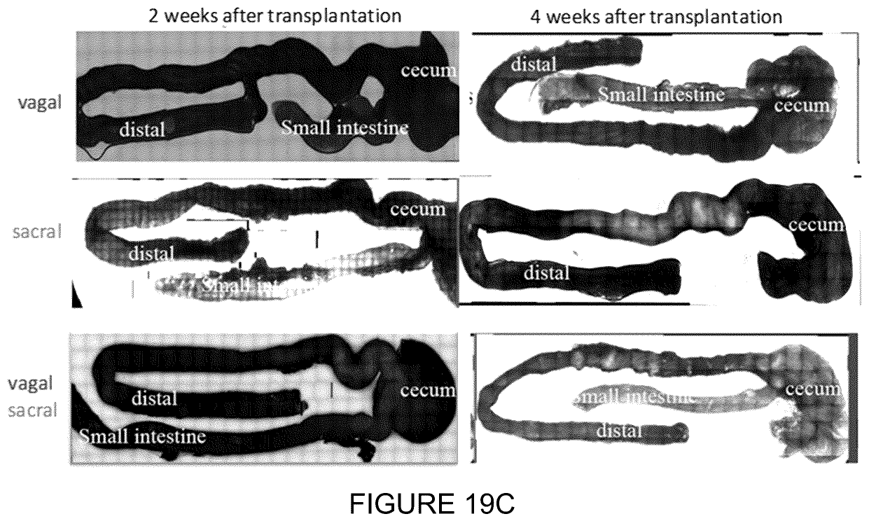

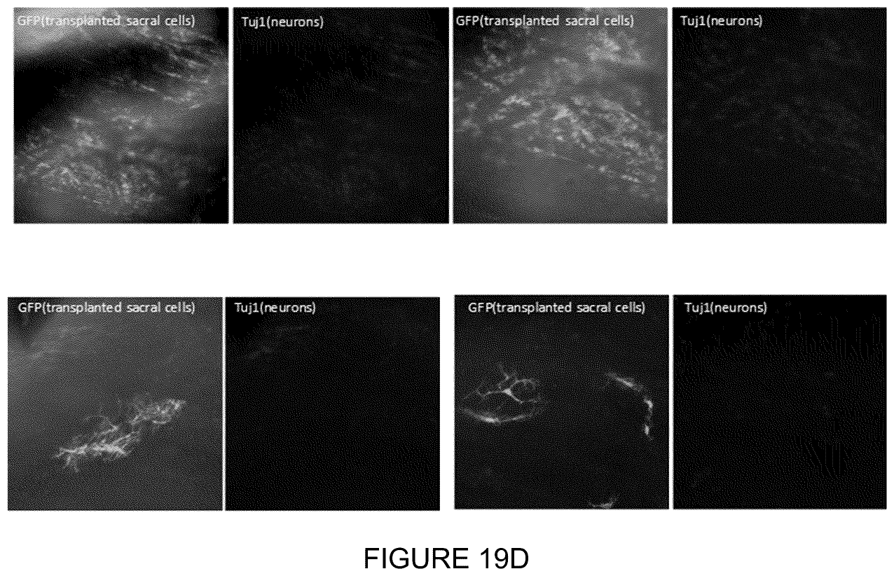

Differences between VNCs and SNCs were also analyzed in vivo. Experiments were conducted to assess the migratory behaviors of VNCs and SNCs in vivo. Briefly, fluorescence labeled cells were injected into the colons of mouse models and were analyzed after transplantation (FIGS. 19A and 19B). The VNCs and SNCs showed different migratory behaviors in vivo (FIG. 19C). Further analysis in whole mount staining showed that SNCs differentiated into neurons and glia cells in the mouse colon (FIG. 19D), implying their potential use for cell therapies. Thus, VNC and SNC showed distinct migratory behaviors both in vivo and in vitro.

### Example 6: Rescuing HD Mice with VNCs and SNCs

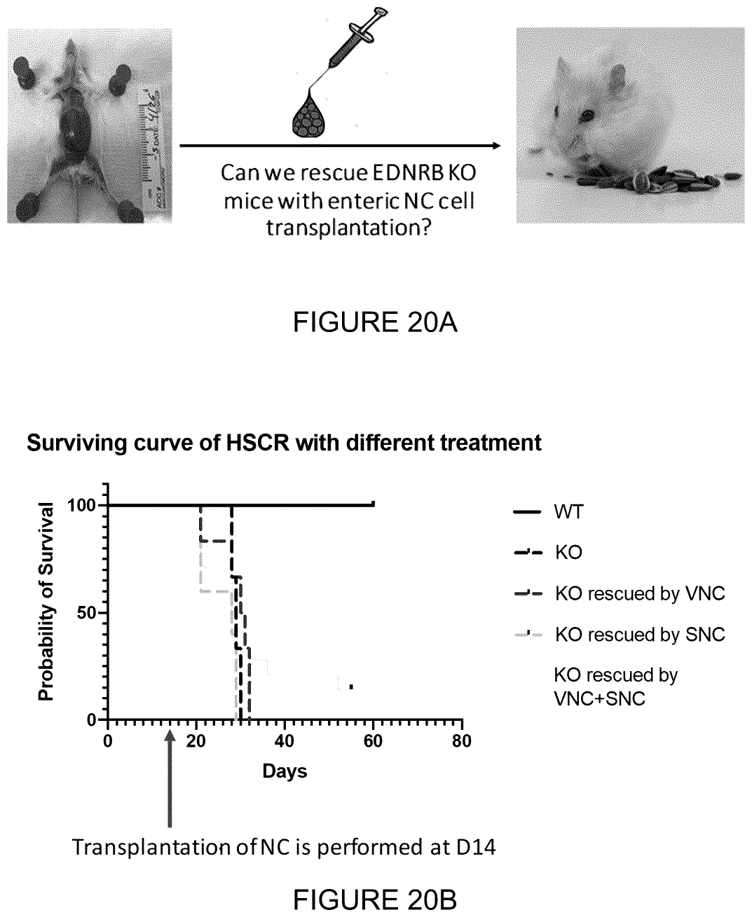

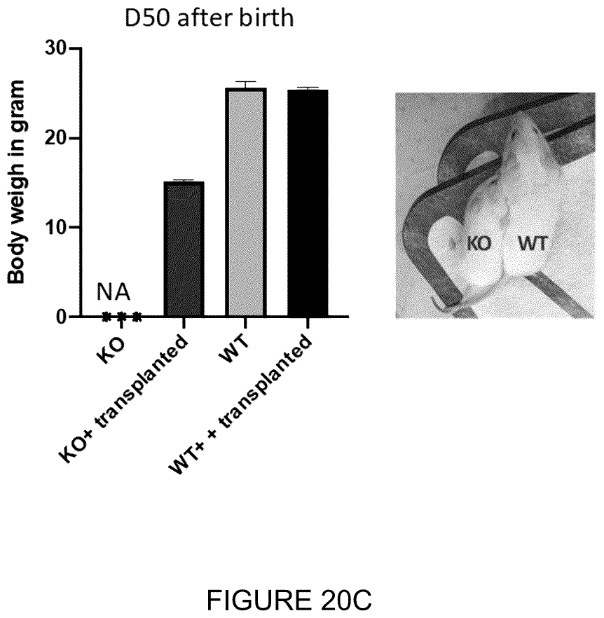

To validate the use of enteric NCs for therapeutic purposes, rescue experiments were performed by transplanting enteric NC cells into a Hirschsprung's disease mouse model (EDNRB knockout mouse, FIG. 20A). As shown by the survival curves in FIG. 20B, the combination of VNCs and SNCs extended the lifespan of some mice from 28 to 55 days. Notably, SNCs and VNCs when used alone did not have any significant effect on the lifespan of the animals. The combination of VNCs and SNCs also affected bodyweights of the animals (FIG. 20C). Thus, the transplantation of VNC and SNC alone did not show any rescue effect, while their combination showed partial rescue of the Hirschsprung's mouse models.

### Example 7: Exemplary Protocol for Derivation of Sacral Neural Crest Lineage Cells from Human Pluripotent Stem Cells

This Example describes an exemplary protocol for producing sacral neural crest lineage cells. Before the induction of sacral NC cells, the hPSC cells were kept in a chemically defined and feeder free culture system named E8. The hPSCs were cultured in the 10-cm dishes, which were coated with Vitronectin. 10 ml of E8 media were added to each plate and the media were changed every day. When the cells reached around 70% confluency, EDTA was used to dissociate the cells and the cells were passed at a 1:15 ratio.

From Day 0 to Day 1, the cells were cultured in an induction medium A. The induction medium A was an E6 medium comprising 100 ng/ml of FGF2, 3 μM of CHIR99021, 1 ng/ml of BMP4, and 2 μM of SB431542. The cells were fed every day with 2 ml induction medium per 24 well.

From Day 1 to Day 4, the cells were cultured in an induction medium B. The induction medium B was an E6 medium comprising 100 ng/ml of FGF2, 3 μM of CHIR99021, 1 ng/ml of BMP4, and 50 ng/ml of GDF11. The cells were fed every day with 2 ml induction medium per 24 well.

From Day 5 to Day 20, the cells were cultured in an induction medium C. The induction medium C was an E6 medium comprising 10 μM of SB431542, 1.5 μM of CHIR99021, and 1 ng/ml of BMP4. The cells were fed every other day with 2 ml induction medium per 24 well.

Details are shown in FIG. 21.

### Example 8: hPSC-Derived Sacral Neural Crest Enables Rescue in a Severe Model of Hirschsprung's Disease

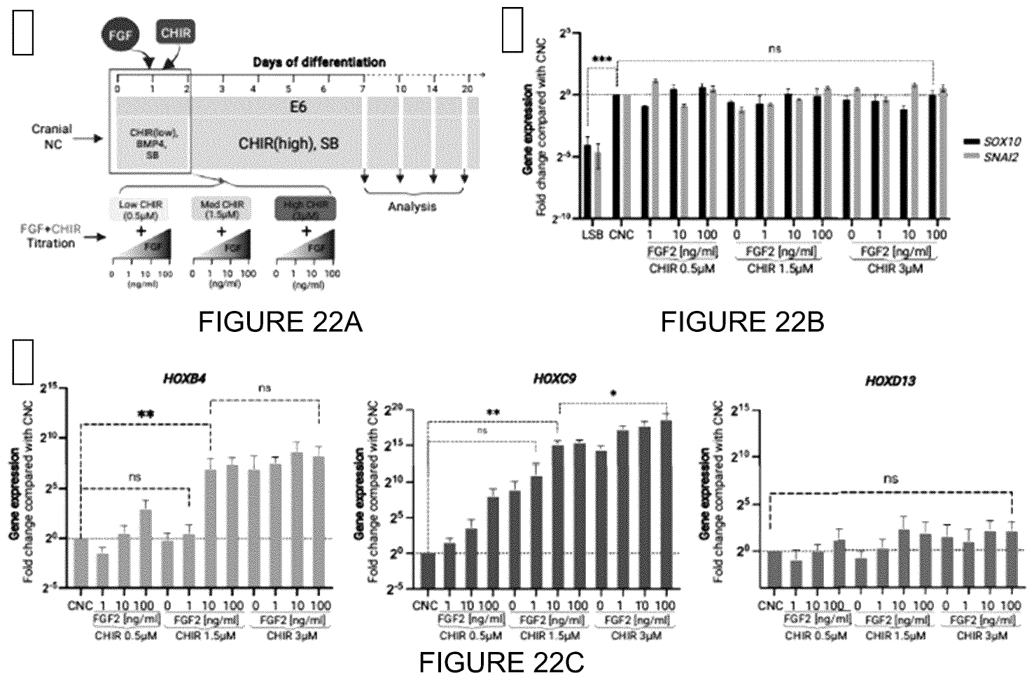

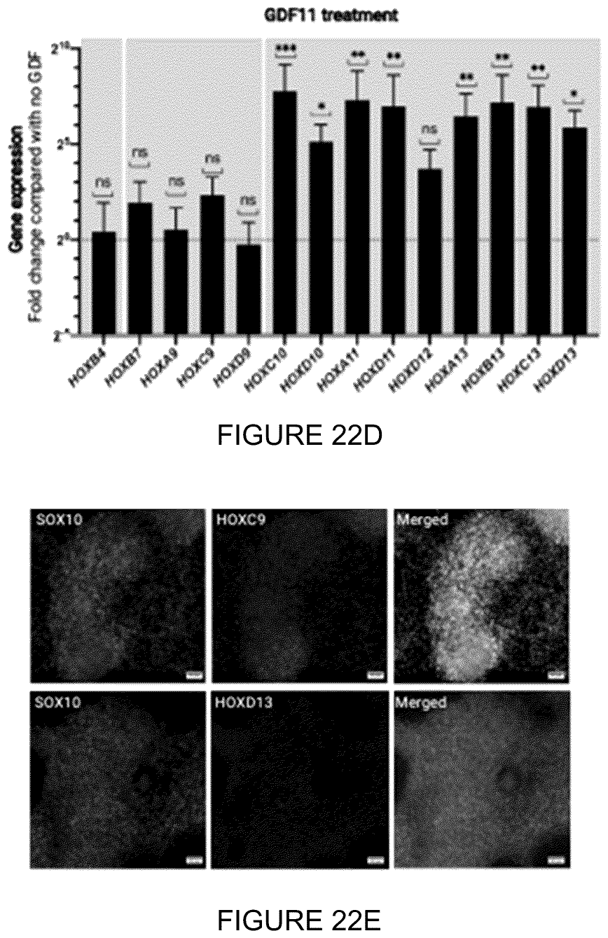

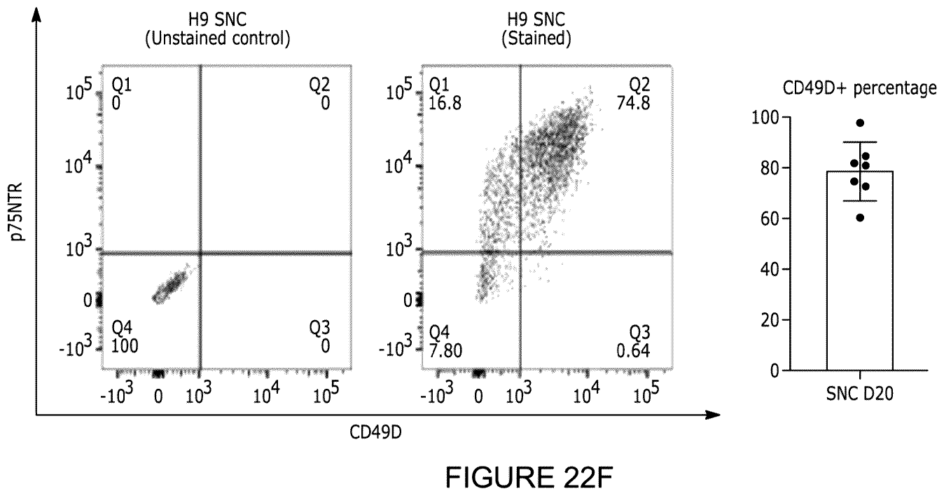

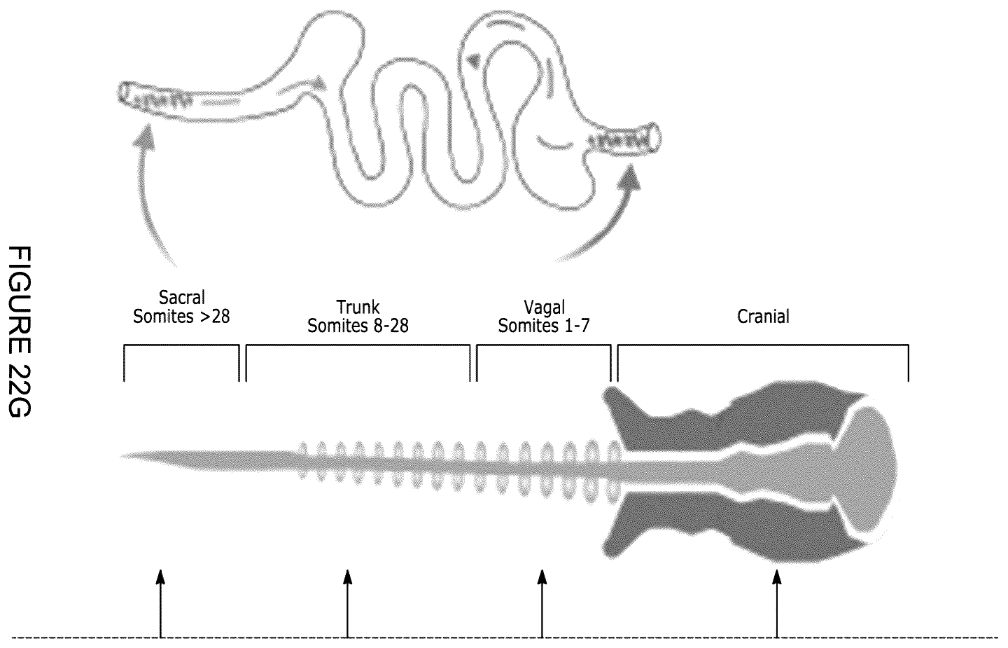

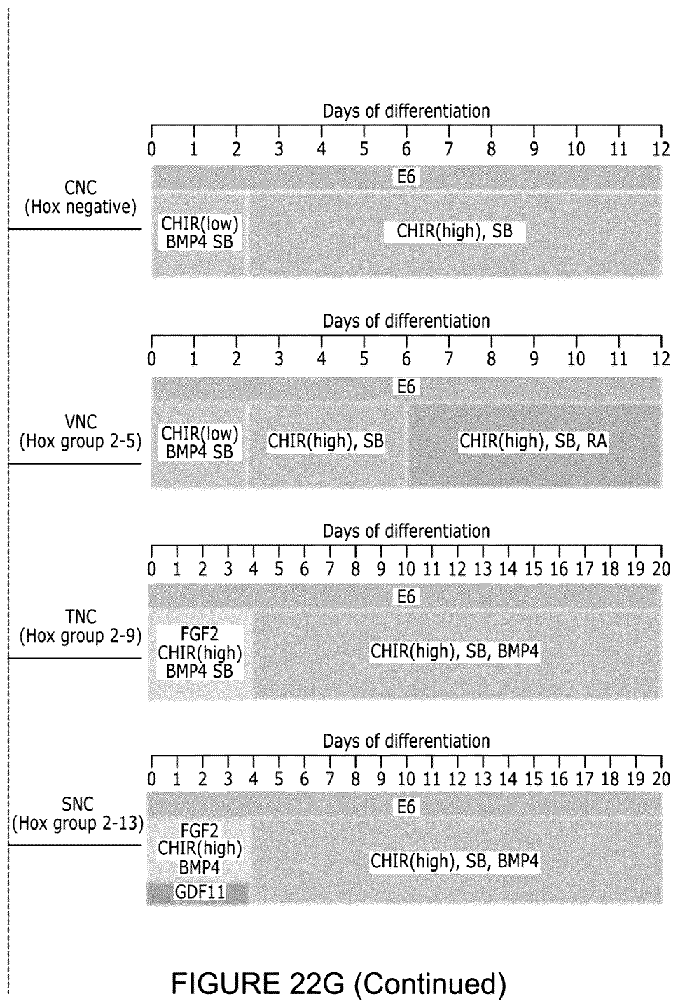

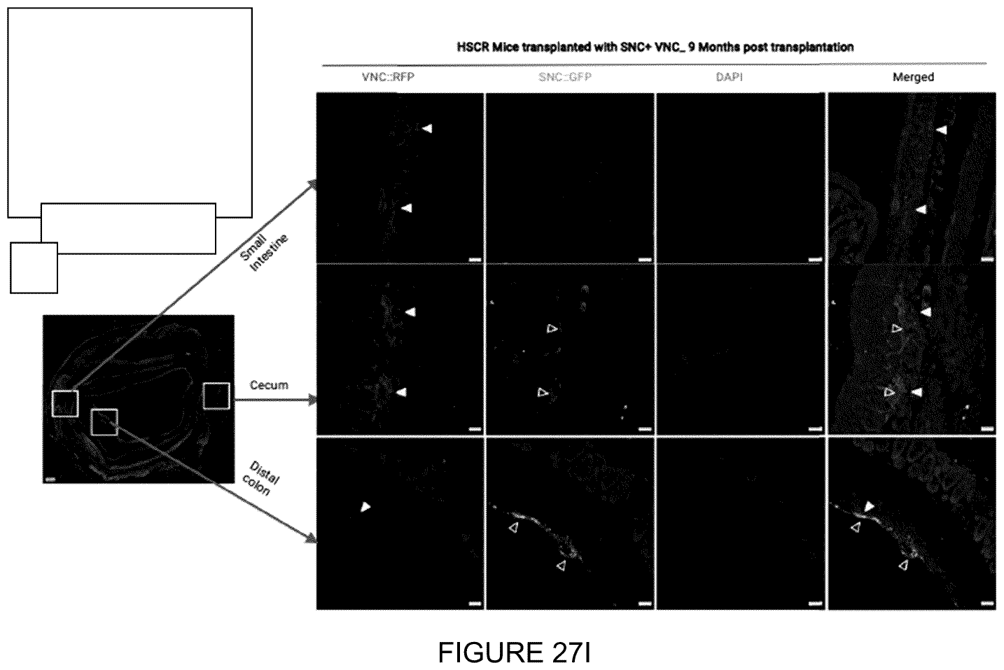

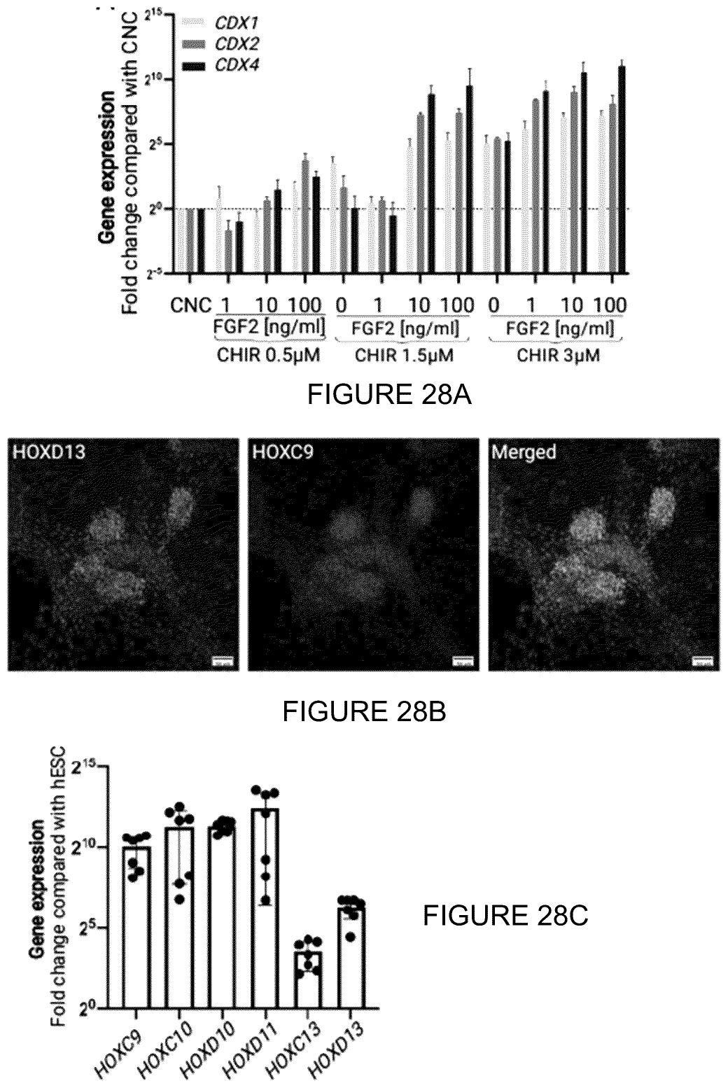

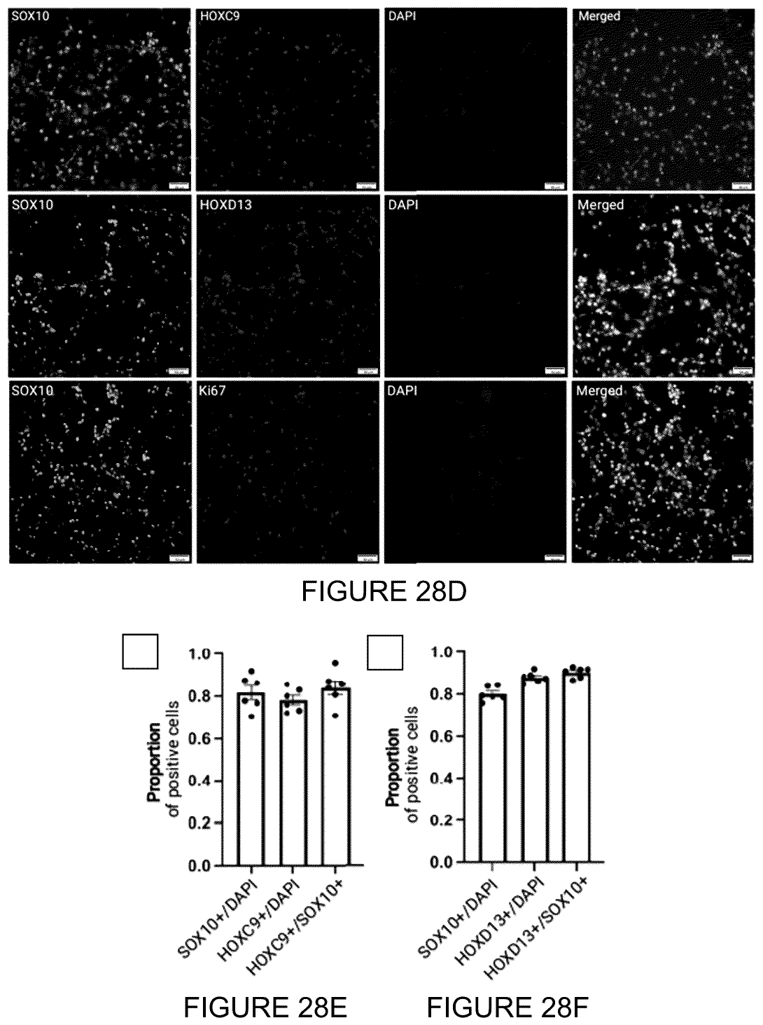

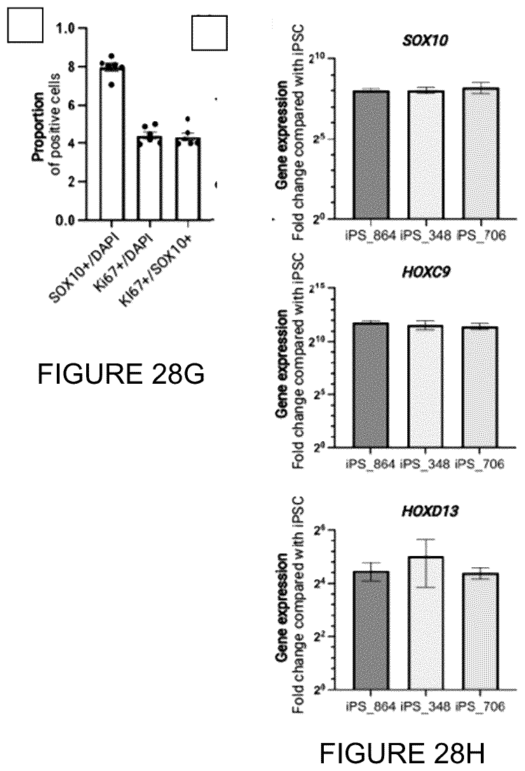

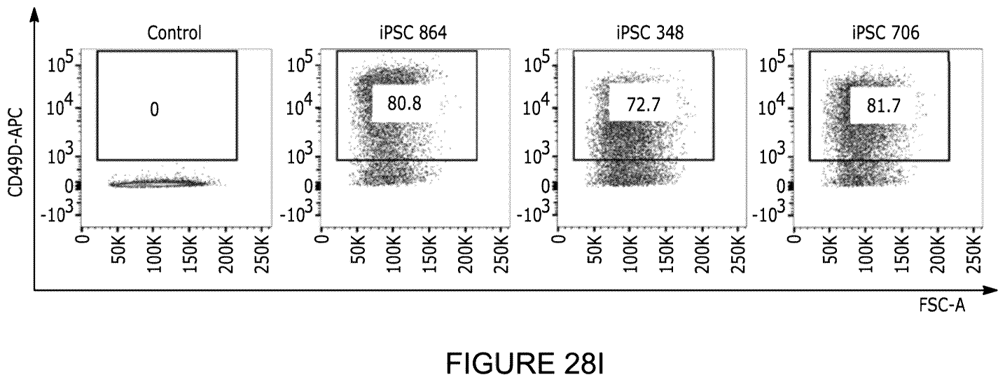

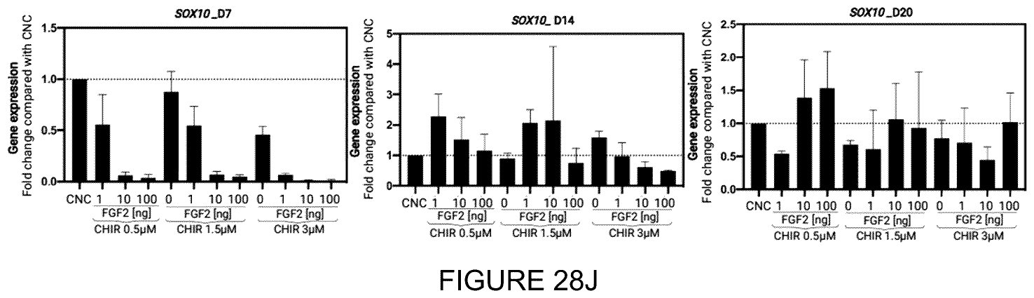

Derivation of sacral NC from hPSCs. During the development, the NC is generated along the anterior to posterior (AP) axis giving rise to cranial, vagal, trunk and sacral NC respectively. Each NC domain shows distinct lineage potential and is characterized by the expression of distinct HOX genes (Rothstein et al., Dev Biol 444 Suppl 1, S170-S180, 2018). To direct differentiation into sacral NC lineages, candidate patterning factors, including modulators of FGF, WNT and RA signaling, the key pathways known to drive posterior fates during embryonic development were screened (FIG. 22A). By monitoring both HOX gene expression and expression of the key NC markers SOX10 and SNAI2, it was found that early exposure to activators of WNT and FGF signaling triggers robust expression of trunk level HOX gene expression without interfering with the expression of NC markers (FIGS. 22B and 22C). Treatment with CHIR99021 (CHIR) and FGF2 further induced expression of HOX regulatory genes such as the CDX transcription factors (CDX1, CDX2, CDX4; FIG. 28A). However, despite robust induction of trunk level HOXgenes, sacral HOX genes were not expressed under any of the conditions tested (FIG. 22C, right panel). Exposure to RA, in addition to FGF2 and CHIR, did not increase expression of either trunk or sacral level HOX genes (data not shown). Those data suggest that additional patterning factors are required to induce sacral NC.

GDF11 was studied as such a candidate factor. GDF11, also known as BMP11, is a TGFβ family member expressed in the posterior neural tube and tailbud mesoderm (McPherron et al., Nat Genet 22, 260-264, 1999; Nakashima et al., Mech Dev 80, 185-189, 1999). GDF11 has been implicated in the trunk to tail transition with Gdf11 KO mice exhibiting extended trunk and reduced hindlimb and tail structures (Jurberg et al., Dev Cell 25, 451-462, 2013; Liu, 2006; McPherron et al., Nat Genet 22, 260-264, 1999; Suh et al., J Cell Physiol 234, 23360-23368, 2019; Szumska et al., Genes Dev 22, 1465-1477, 2008). It was observed that early exposure to GDF11, in combination with FGF2 and CHIR treatment triggered a dramatic and selective increase in sacral HOX gene expression (HOX10-13) without affecting the expression of the more anterior HOXgenes (HOX4-9) (FIG. 22D). Sacral level HOXgene expression did not interfere with NC induction as illustrated by the co-expression of HOXC9 and HOXD13 with SOX10 (FIG. 22E) and co-expression of HOXC9 and HOXD13 in GDF11 treated cultures (FIG. 28B). Due to the high cell density and the formation of crest-like ridges during NC induction, it was difficult to precisely quantify the extent of co-expression of SOX10 with posterior HOX genes. Therefore, GDF11-treated cultures were dissociated at D20 followed by replating cells as a monolayer and characterized by qRT-PCR (FIG. 28C) and by immunocytochemical analysis for co-expression of SOX10 with HOXC9, HOXD13 and Ki67 (FIGS. 28D-28G). Those data confirmed sacral NC identity and indicated that most of the cells are proliferative (Stauffer et al., Scientific Reports 8, 15764, 2018). To validate robustness of the sacral NC protocol, multiple independent hESC and hiPSC lines were assessed for HOX gene expression by qRT-PCR and for NC lineage markers via flow cytometry for p75NTR and CD49D⁺ (Fattahi et al., Nature 531, 105-109, 2016)(FIGS. 22F, 28H, and 28I). These data demonstrate that we have established a robust protocol for the induction of trunk and sacral NC. In combination with the work on cranial and vagal NC (Fattahi et al., Nature 531, 105-109, 2016; Tchieu et al., Cell Stem Cell 21, 399-410 e397, 2017; Zeltner et al., JoVE (Journal of Visualized Experiments), e51609, 2014), the presently disclosed subject matter offers modular access to all four NC domains for studies of human NC development and for cell fate diversification across most NC-derived lineages (FIG. 22G).

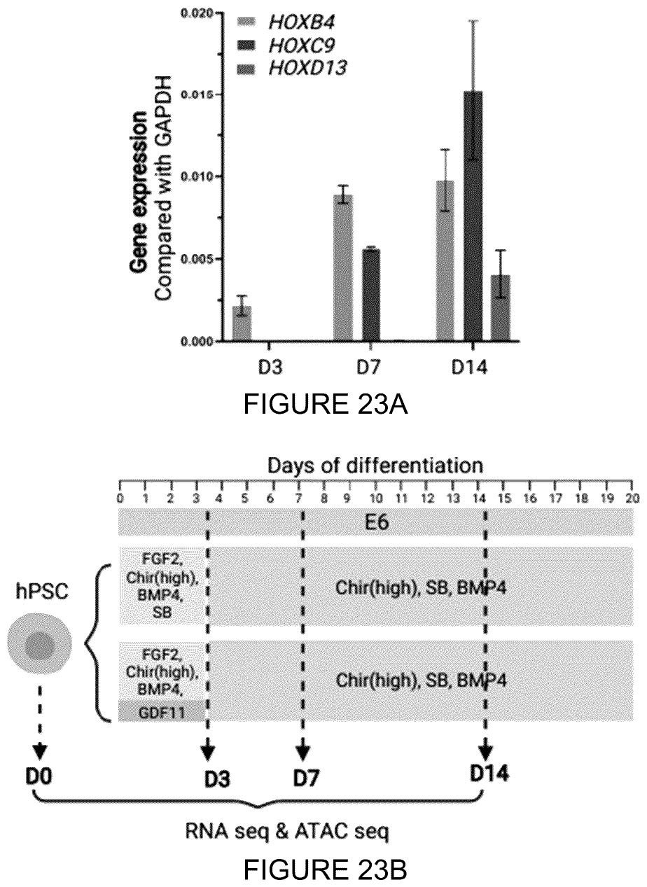

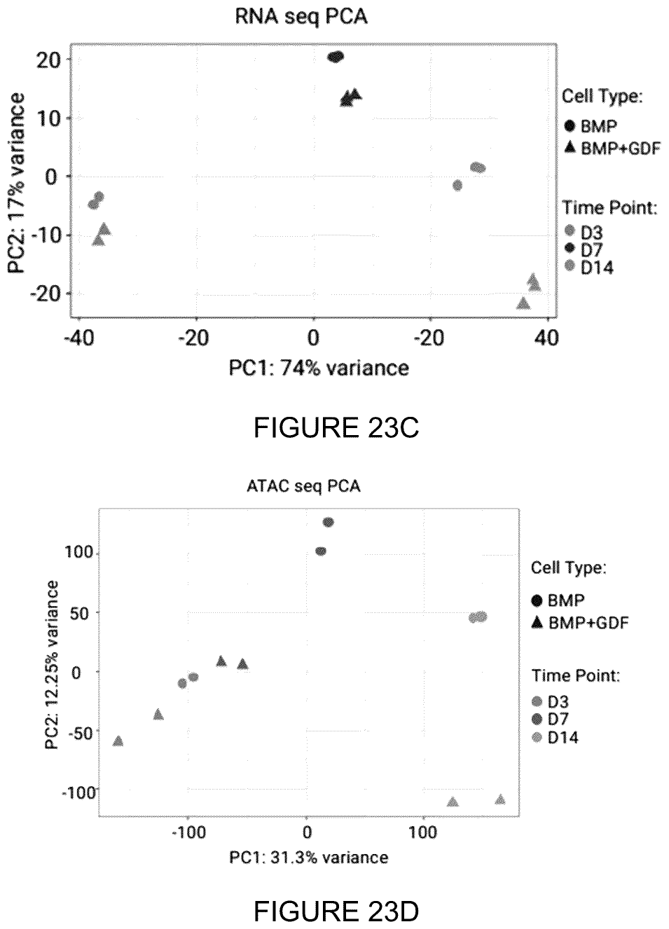

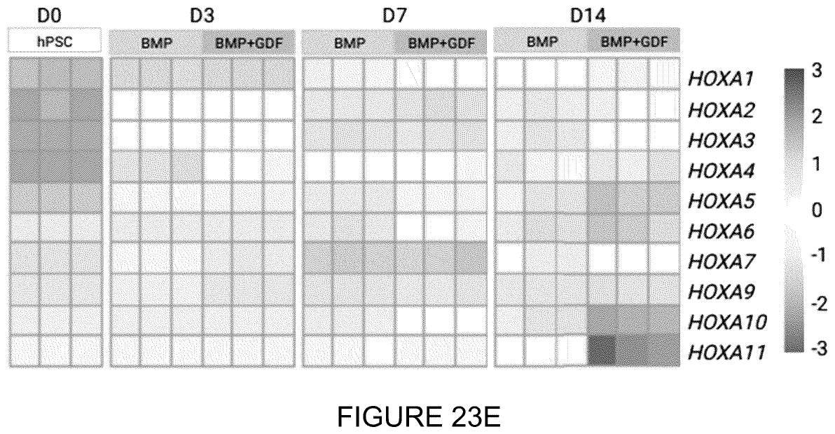

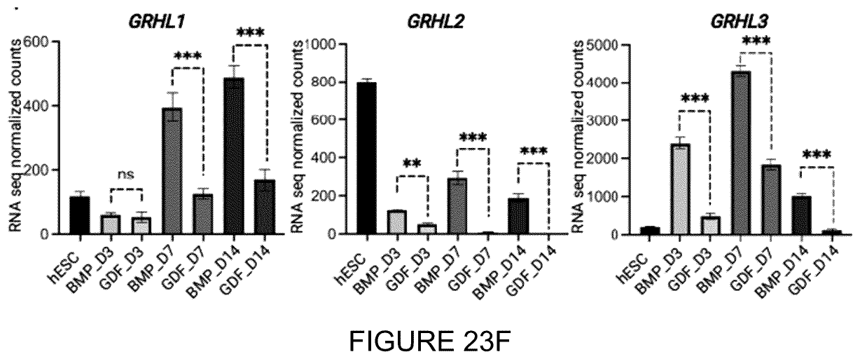

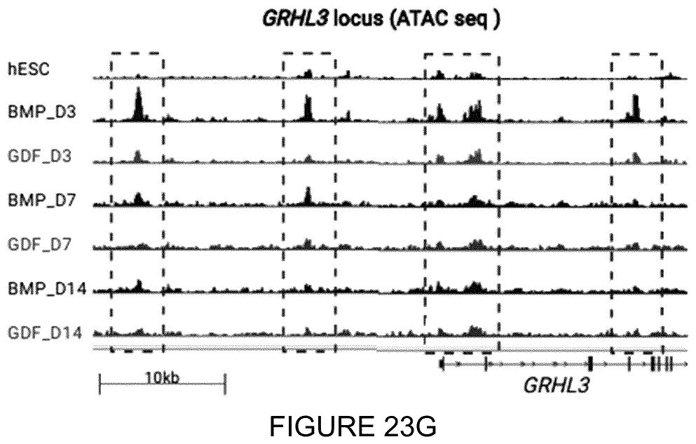

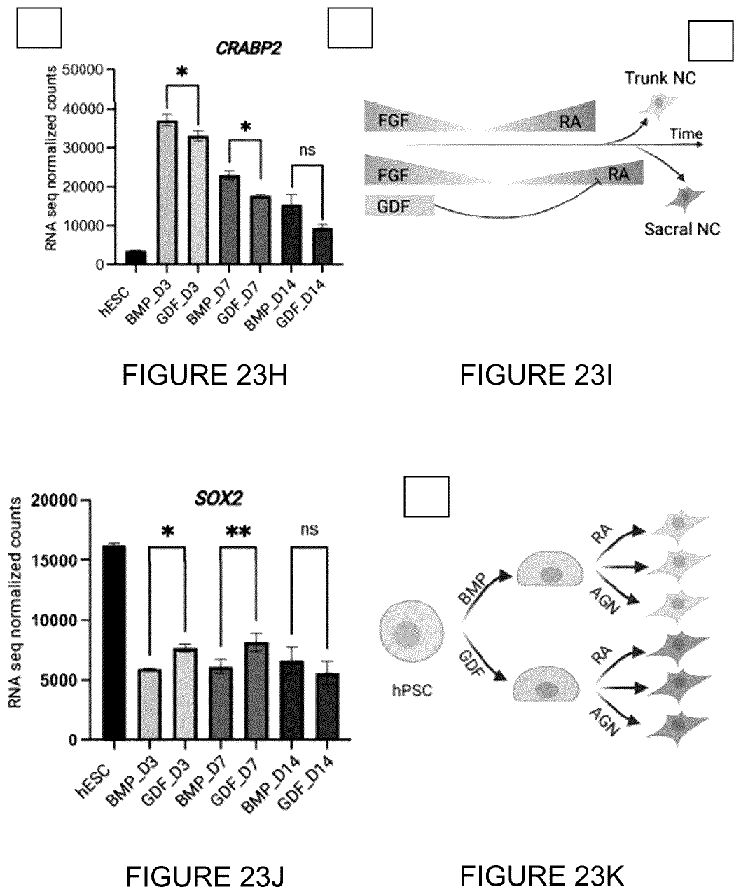

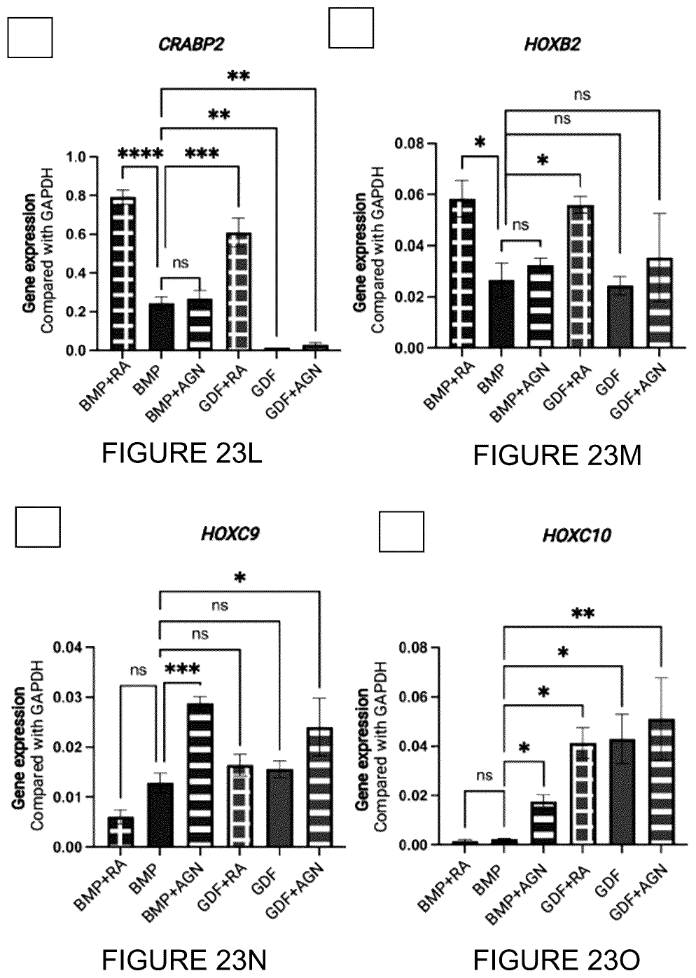

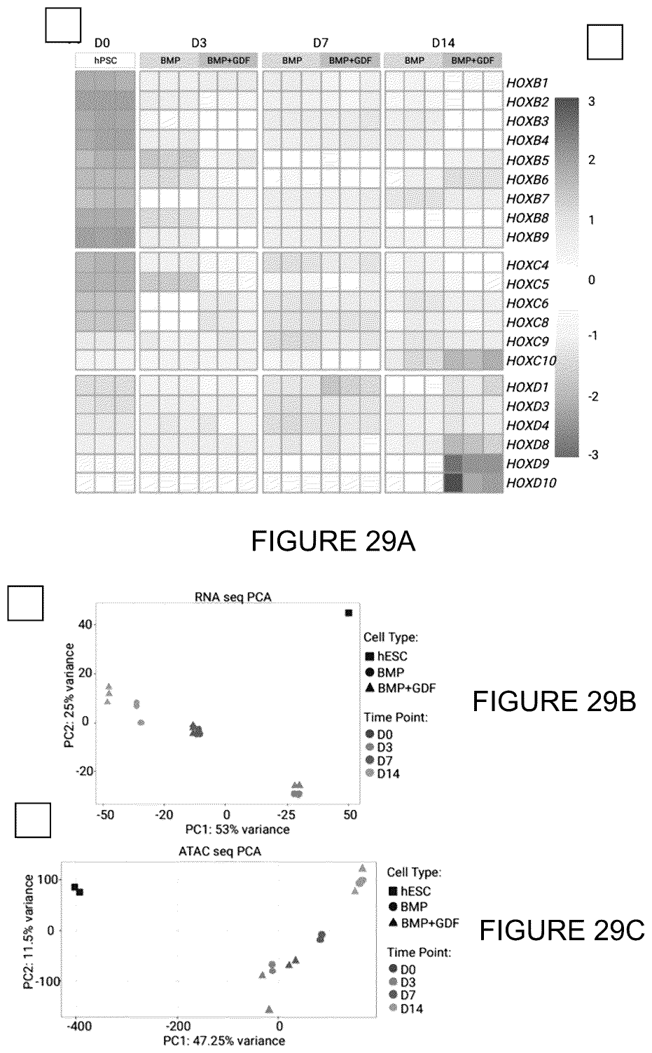

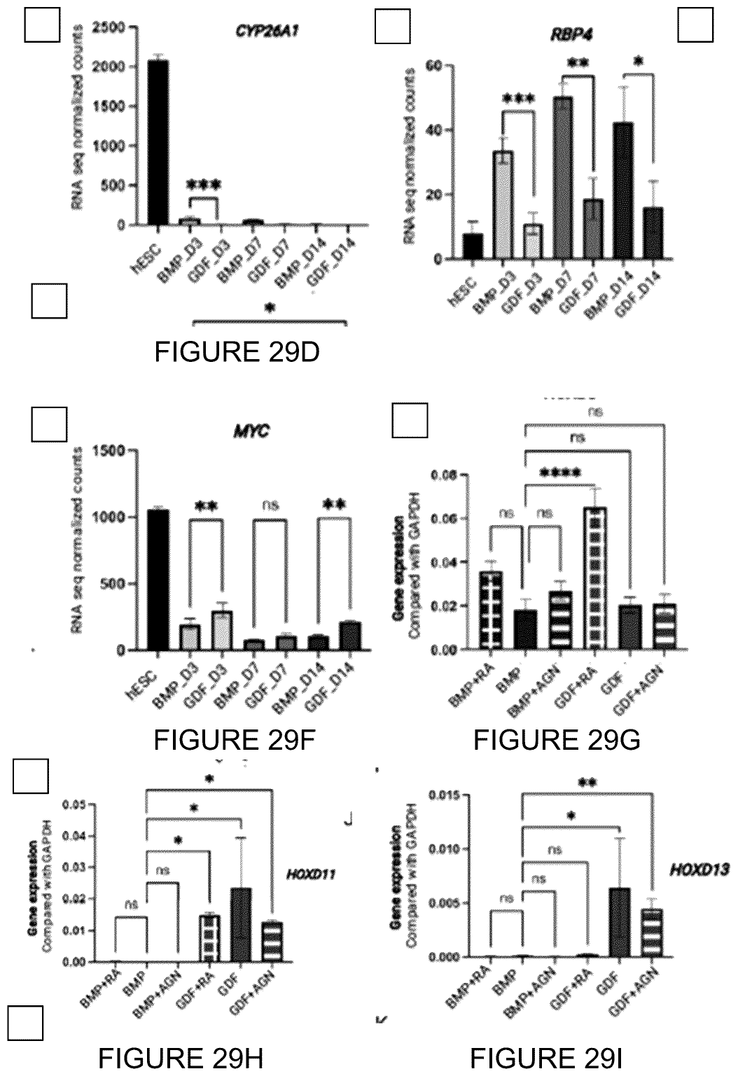

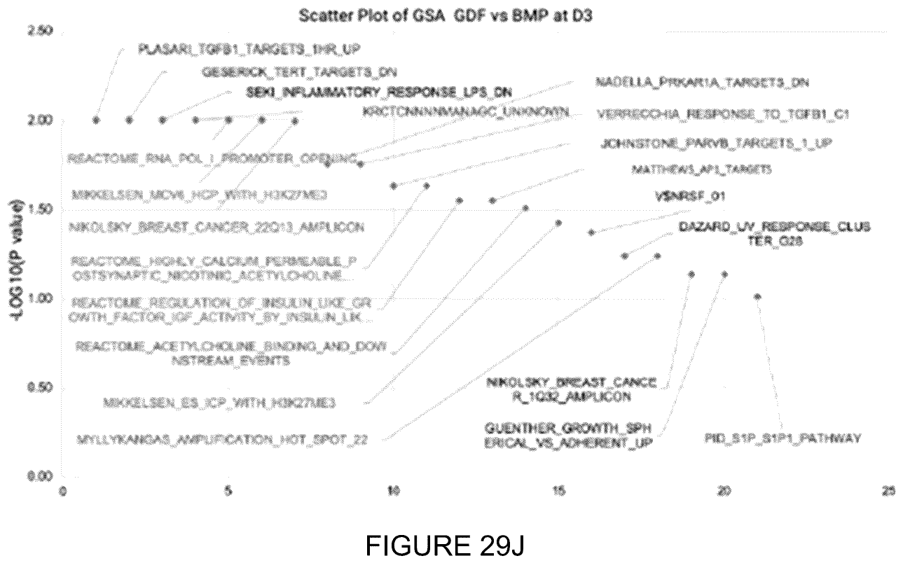

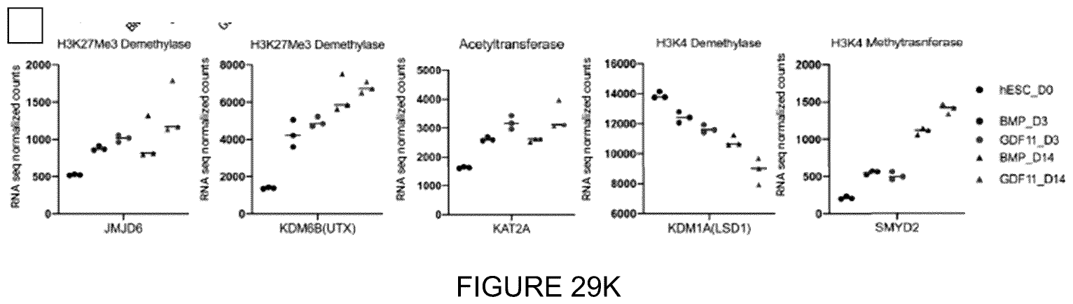

GDF11-mediated expression of 5′ HOX genes via modulation of RA signaling. The temporospatial sequence of HOX gene expression along the AP axis is evolutionarily conserved (Akam, Development 101, 1-22, 1987: Burke et al., Development 121, 333-346, 1995). Co-linearity in HOX gene expression from 3′ to 5′ HOX genes is also maintained in the presently disclosed hPSC platform in vitro with 3′ HOXgenes such as HOXB4 induced first, followed by trunk level genes such as HOXC9 and the expression of the most 5′ HOX genes including HOXD13 (FIG. 23A). Given the key role of GDF11 for inducing the most 5′ HOX genes (FIG. 221)), it was next explored how GDF11 treatment affects gene expression and chromatin accessibility during NC differentiation (FIGS. 23B and 29A). Principal component analysis (PCA) of the RNAseq and ATACseq data showed time (day of differentiation) as the main driver of variance. (FIGS. 23C, 23D, 29B, and 29C). Nevertheless, cultures treated with GDF11 showed a clear segregation within PCA space at both D3, D7 and at D14 of differentiation. Heat map analysis of HOX gene expression at sequential time points of differentiation confirmed the progression from anterior (3′) to posterior (5′) HOX genes (FIGS. 23E and 29A). Next, RNAseq and ATACseq data were integrated to identify candidate genes that may mediate GDF11 action at the level of both gene expression and chromatin accessibility. One class of genes coordinately regulated by GDF 1I were the GRHL transcription factors (GRHL1, GRHL2 and GRHL3), which showed a significant decrease following GDF11 exposure for both gene expression (FIG. 23F) and chromatin accessibility (FIG. 23G). GRHL factors have been previously shown to regulate pathways relevant to HOX gene expression including retinoic acid signaling (Seller et al., Proceedings of the Royal Society of London. Series B. Biological Sciences 206, 95-107, 1979; van den Berg et al., Cell Stem Cell 6, 369-381, 2010) and epigenetic modifications such as methylation of H3K27 (Chen et al., J Biol Chem 285, 40852-40863, 2010) and H3K4 (Hopkin et al., PLoS Genet 8, e1002829, 2012). During mouse development, retinoic acid signaling can trigger anterior transformation of axial populations and truncation of the tail structures, a phenotype exacerbated in Gdf11 KO mice and partially rescued by inhibiting RA signaling (Lee et al., Dev Biol 347, 195-203, 2010). Interestingly, the expression of key mediators and direct targets of RA signaling such as CRABP2 (Delva et al., Mol Cell Biol 19, 7158-7167, 1999; Ghaffari and Petzold, Theoretical biology & medical modelling 15, 16-16, 2018) was reduced following GDF11 treatment (FIG. 23H) together with other regulators of RA signaling including CYP26A1 and RBP4 (Ghaffari and Petzold, Theoretical biology & medical modelling 15, 16-16, 2018) (FIGS. 29D and 29E). A previous study on trunk NC development in the mouse showed that FGF signaling prevents, and RA promotes, premature EMT and NC induction (Martinez-Morales et al., Journal of Cell Biology 194, 489-503, 2011). Therefore, without being bound by any particular theory, GDF11 treatment may act by reducing RA signaling and thereby maintaining axial progenitors, which in turn allows those progenitors to reach sacral HOX gene levels prior to depletion of the progenitor pool (FIG. 23I). This hypothesis is supported by the increased expression of stem cell-related factors such as SOX2 (FIG. 23J) and MYC (FIG. 29F) in GDF11 treated cultures. To directly test this hypothesis, the presently disclosed subject matter assessed how RA signaling affects the patterning of trunk (BMP only) versus sacral (BMP+GDF11) NC (FIG. 23K). First, it was confirmed the expected regulation of RA signaling by monitoring changes in CRABP2 expression (FIG. 23L). Next, it was observed that RA promotes anterior HOX genes including HOXB2 (FIG. 23M) and HOXB6 (FIG. 29G) under both trunk and sacral NC conditions. In contrast, RA reduced the expression of HOXC9. Pharmacological inhibition of RA signaling via exposure to RA inhibitor AGN led to increased HOXC9 expression (FIG. 23N). Most importantly, RA inhibition in trunk NC (BMP condition) promoted the expression of HOXC10 (FIG. 23O) partially mimicking the effect of GDF11 treatment. Finally, RA activation in GDF11 treated cultures shifted expression towards more anterior HOX genes and suppressed HOXC11 and HOXC13 expression (FIGS. 29H and 29I), while RA inhibition in GDF11 treated cultures had minimal impact on sacral HOX gene expression.

Beyond modulating RA signaling GDF11-mediated induction of sacral HOX genes likely involves additional mechanisms. GO analysis of differentially expressed genes at D3 (GDF11+BMP vs BMP) identified multiple terms related to chromatin regulation (highlighted in blue)(FIG. 29J). Previous work showed that GRHL2 overexpression leads to an enrichment for H3K27Me3 via inhibiting the recruitment of histone demethylases (Chen et al., J Biol Chem 285, 40852-40863, 2010). Interestingly, the presently disclosed RNA seq data at day 3 showed increased levels of the H3K27Me3 demethylases JMJD6 and KDM6B and decreased levels of H3K4 methyltransferases such as SMYD2 following GDF11 treatment (FIG. 29K). Those data are consistent with the decreased GRHL levels at day 3 and suggest a potential role for modulating polycomb in addition to modulating RA signaling as mechanisms involved in GDF11-mediated induction of sacral HOX genes.

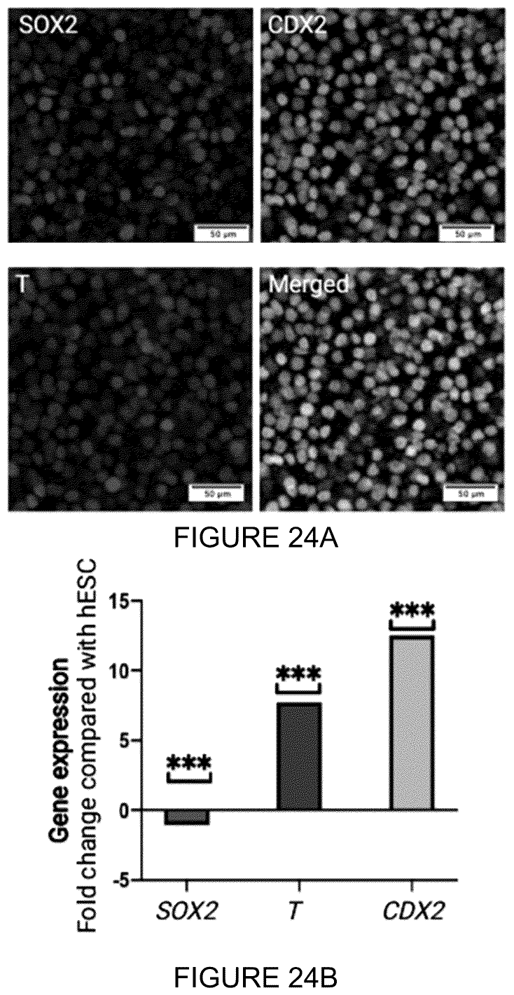

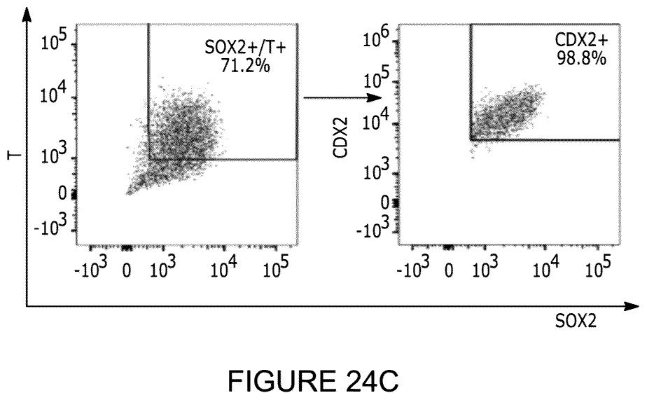

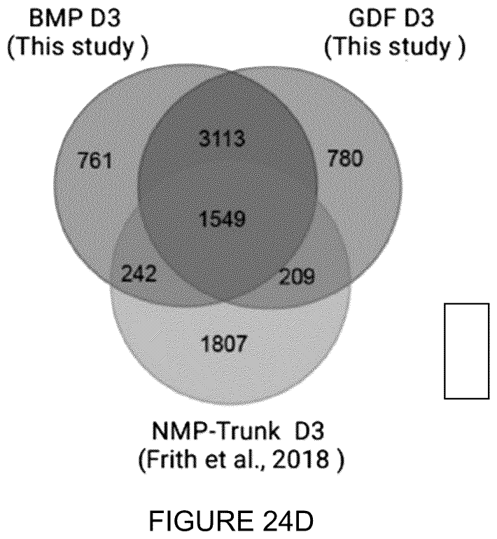

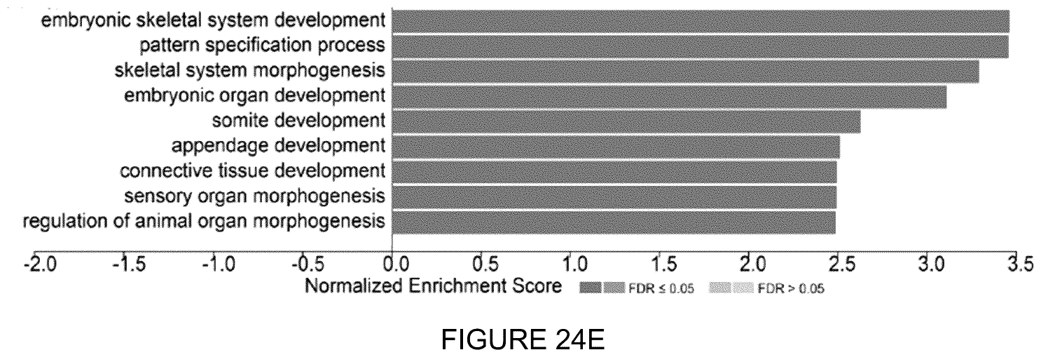

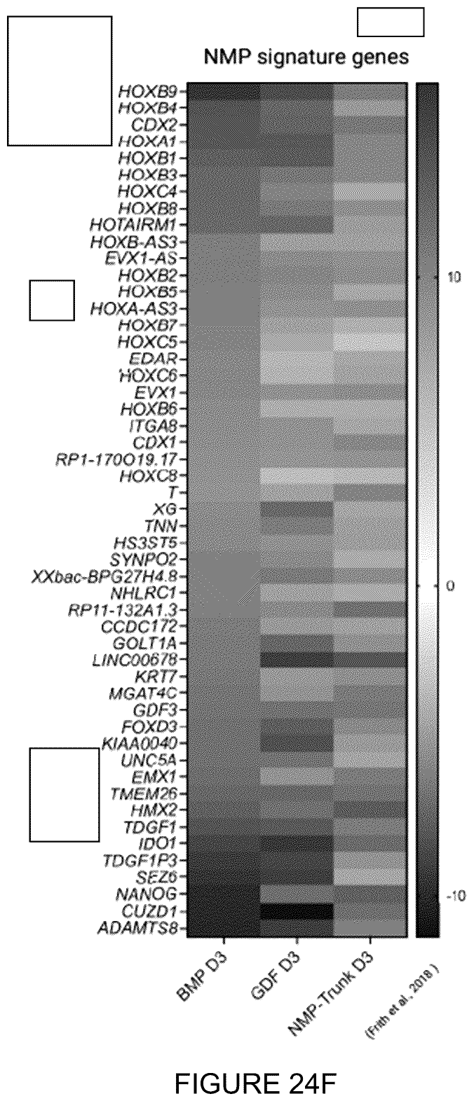

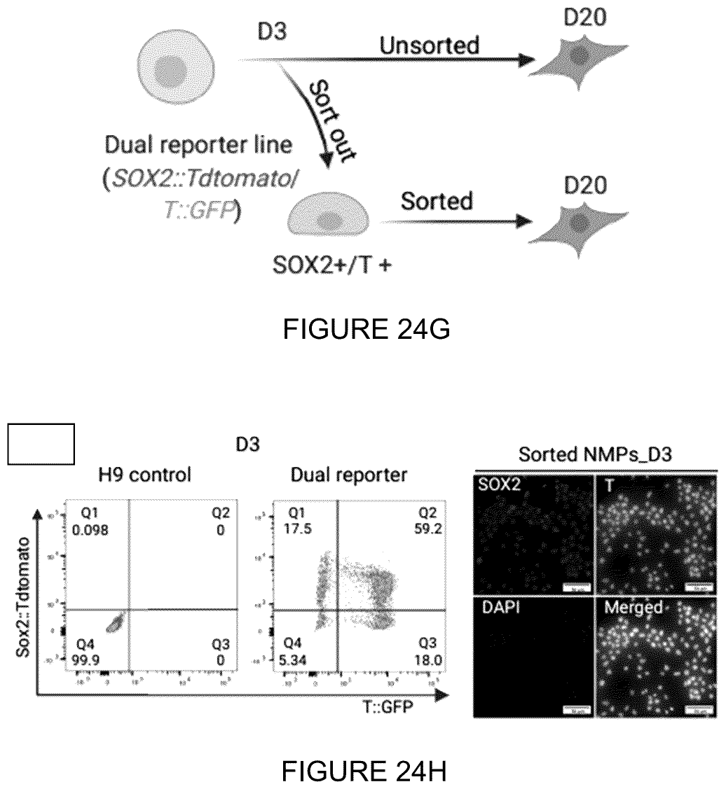

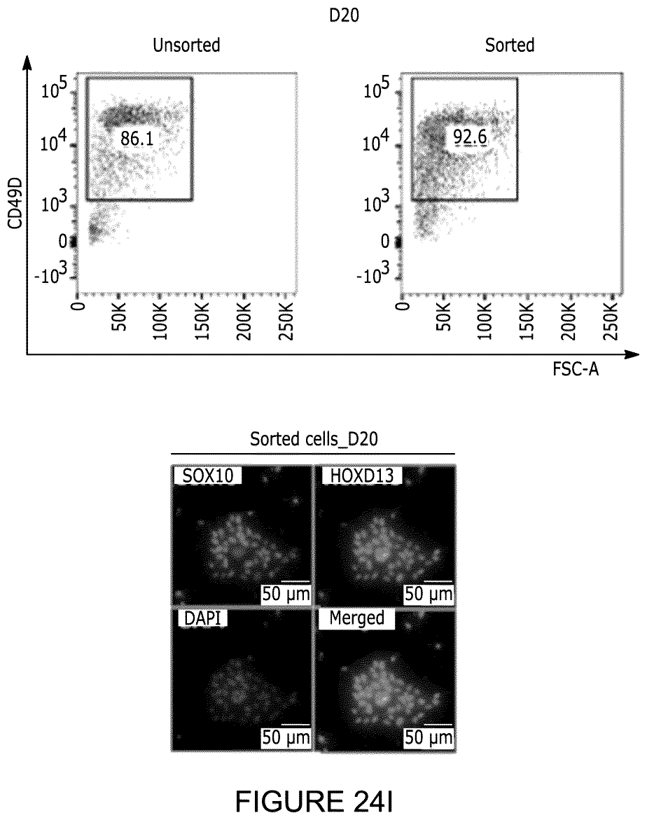

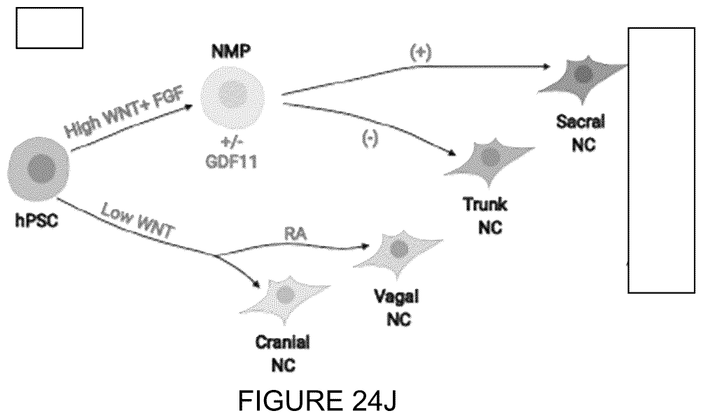

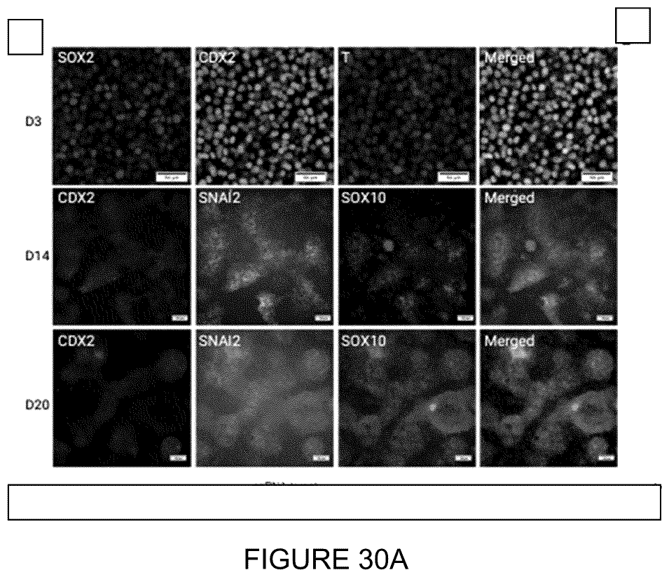

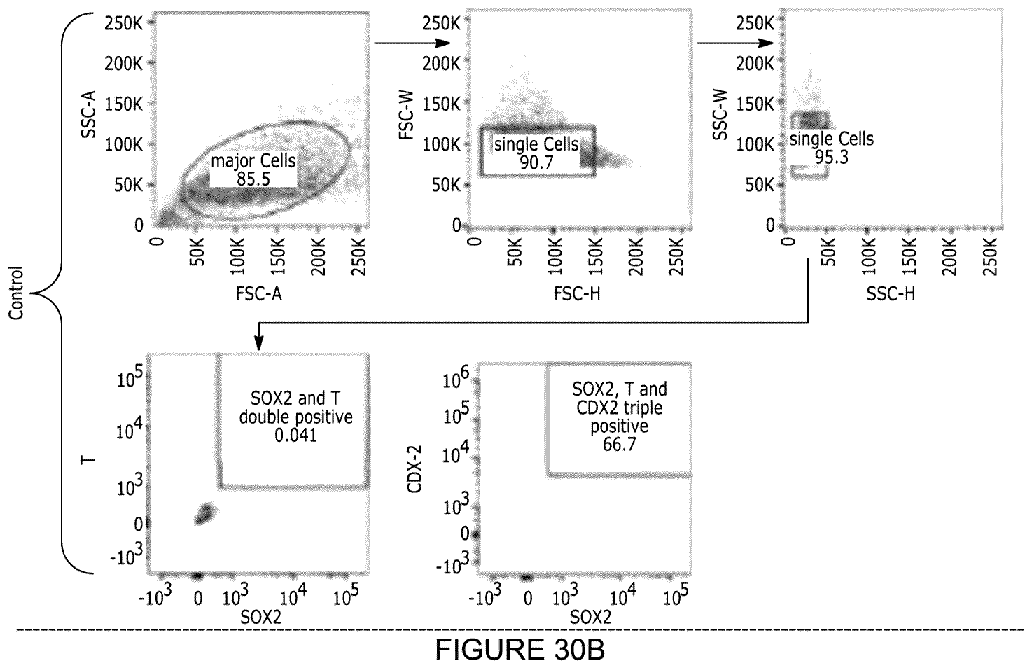

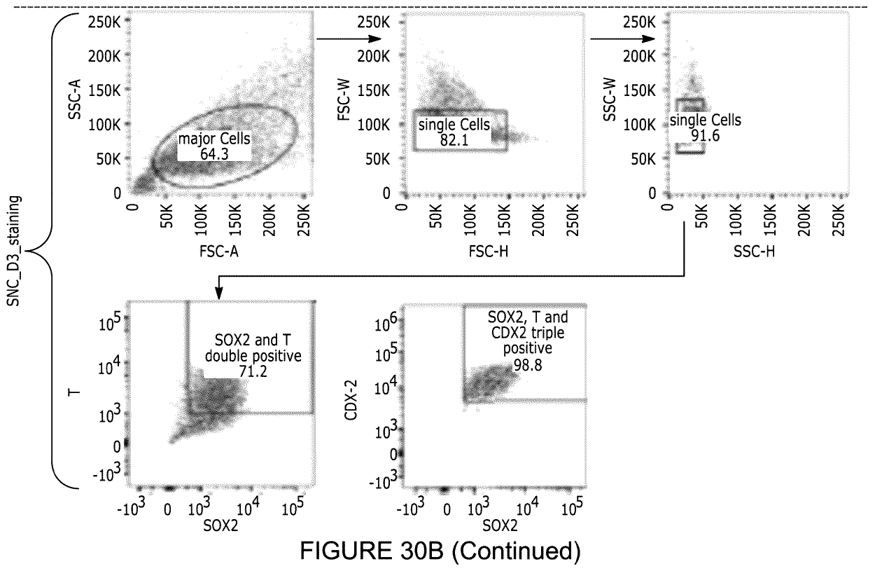

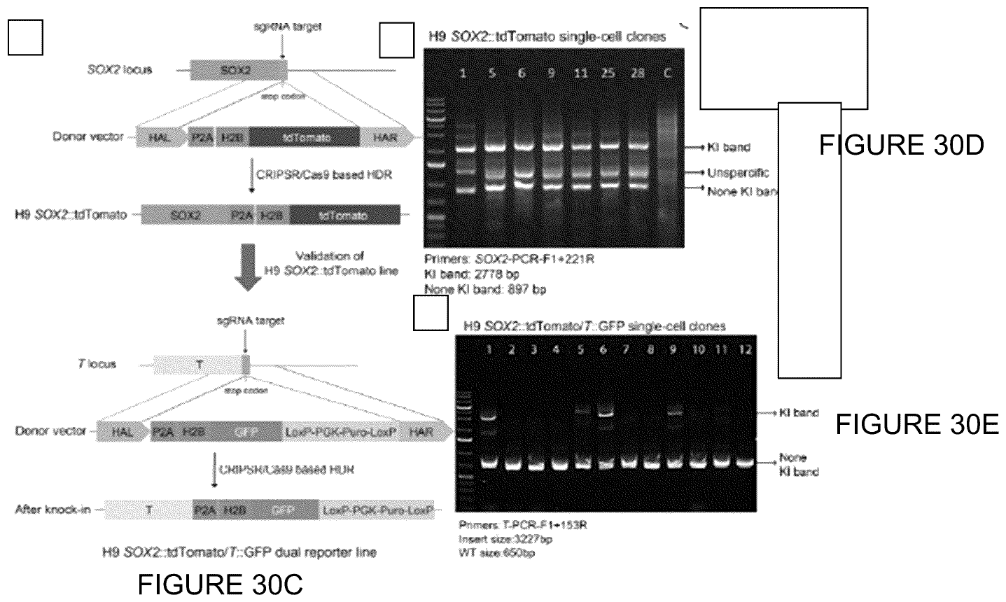

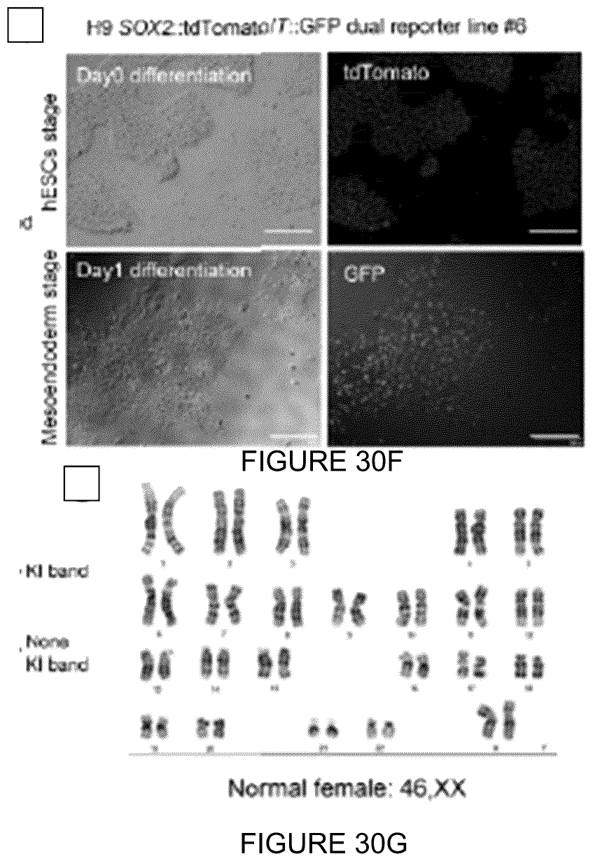

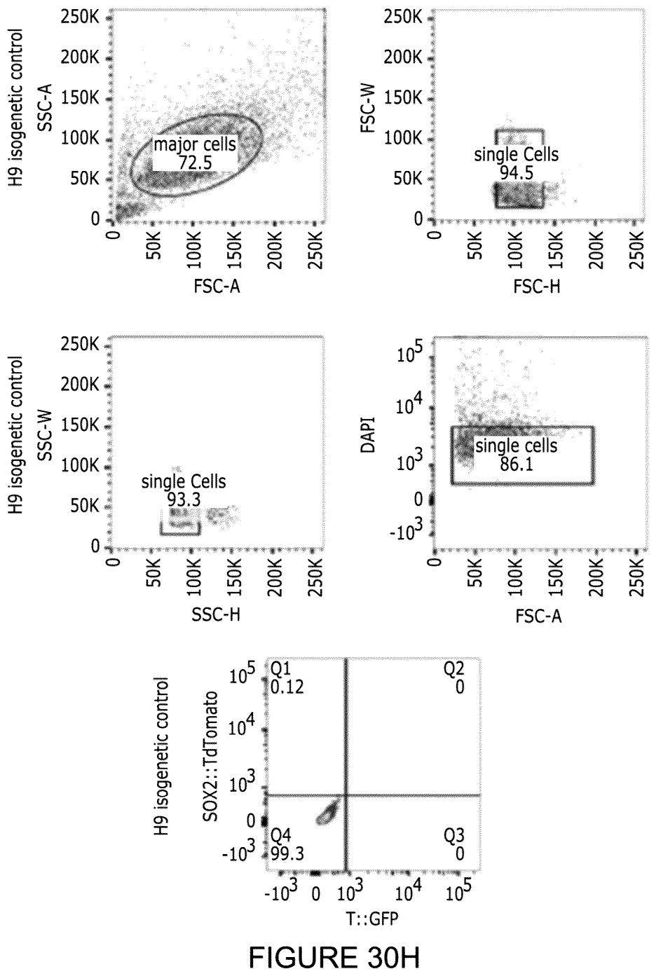

Sacral NC cells are derived from an NMP-like posterior precursor population. There is clear evidence that NC precursors (D6) can adopt vagal instead of cranial identity following RA exposure (Fattahi et al., Nature 531, 105-109, 2016). In contrast, the induction of trunk and sacral NC in the current study is achieved via activation of FGF, WNT and GDF11 signaling at much earlier time points of differentiation (DO-3) prior to the presence of any NC cells. Accordingly, the presently disclosed subject matter postulate that trunk and sacral NC are generated not by caudalizing anterior NC precursors, but by inducing a distinct early precursor state competent to drive posterior HOX gene expression. Indeed, at day D3 of differentiation, it was observed that both the trunk and sacral NC protocols induce a SOX2⁺ precursor population that co-expresses CDX2 and Brachyury (T) at day D3 of differentiation (FIG. 24A), markers characteristic of axial progenitors (Amin et al., Cell Rep 17, 3165-3177, 2016; Cambray and Wilson, Development 129, 4855-4866, 2002; Gont et al., Development 119, 991-1004, 1993; Tzouanacou et al., Developmental cell 17, 365-376, 2009). Gene expression analysis confirmed high levels of SOX2 expression at D3 (comparable to SOX2 levels in hESCs) and robust induction of Brachyury and CDX2 (FIGS. 24B and 30A). FACS analysis of D3 cells showed that around 70% of the cells are SOX2: Brachyury (T) double positive and among those, over 98% percent are triple positive for CDX2 (FIGS. 24C and 30B). RNA seq analysis of D3 cells, patterned using the trunk (BMP) versus sacral (BMP+GDF11) protocol, showed a strong overlap in gene expression and matched the expression reported for hPSC-derived axial progenitors (NMPs) previously (Frith et al., Elife 7.10.7554/eLife.35786, 2018) (FIGS. 24D-24F). These data indicate that both trunk and sacral NC induction protocols may involve a transient NMP-like progenitor stage followed by the progressive induction of NC markers such as SOX10 and SNAI2 from a CDX2′ precursor population by day 14 and day 20 of differentiation (FIG. 30A).

However, while many of the D3 cells are triple positive for SOX2, T, and CDX2, the data do not rule out the possibility that NC precursors are derived from a minor population of non-NMP-like cells. To address this concern, a dual reporter hPSC line for SOX2::H2B-tdTomato; T::H2B-GFP (FIGS. 30C-30G) was established to purify NMP-like cells from contaminating non-NMP populations at D3 followed by further differentiation towards NC lineage (FIG. 24G). FACS-purified NMP-like cells (SOX2⁺/T° cells) at D3 (FIGS. 24H, 30H, and 30I) yielded CD49D⁺ NC cells at very high efficiencies under sacral NC differentiation conditions and with robust co-expression of SOX10 and HOXD13 confirming their sacral NC identity (FIG. 24I). Therefore, these data demonstrate that hPSC-derived NMP-like cells efficiently contribute to sacral NC lineages and suggest that the induction of trunk and sacral NC involves a distinct precursor and differential patterning mechanisms from that of cranial and vagal NC (FIG. 24J).

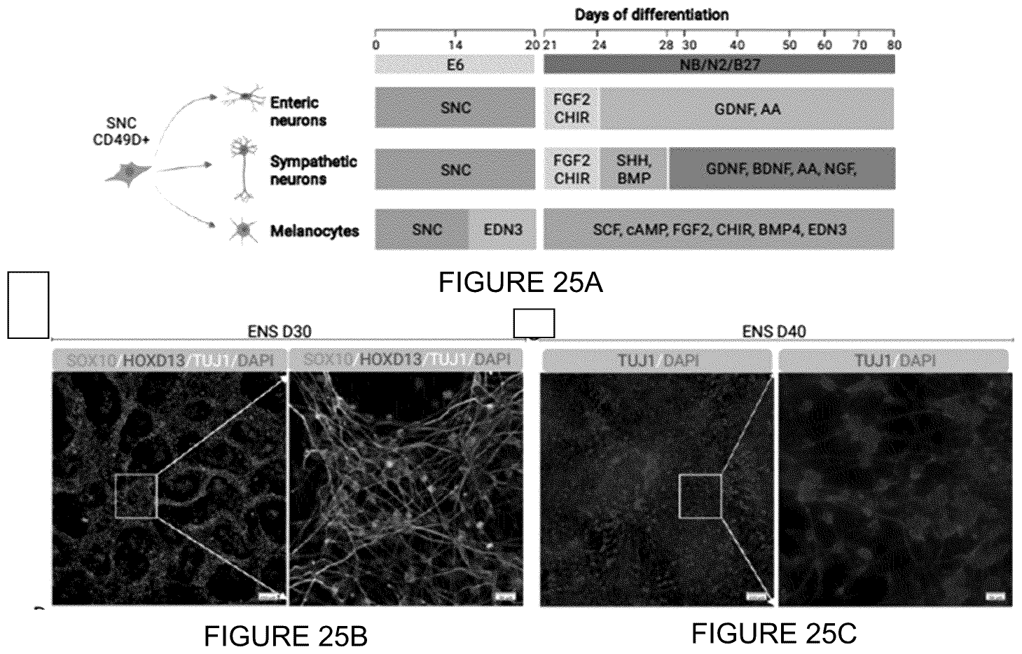

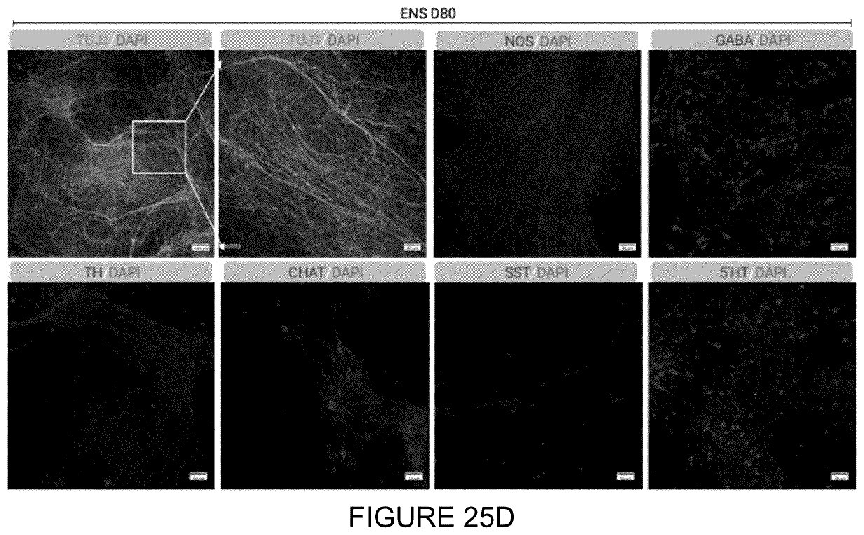

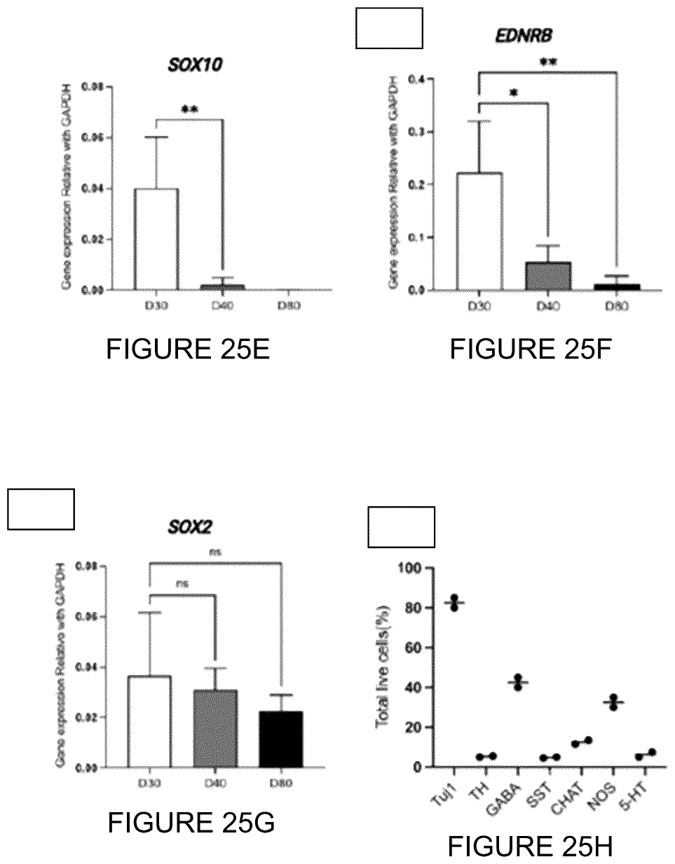

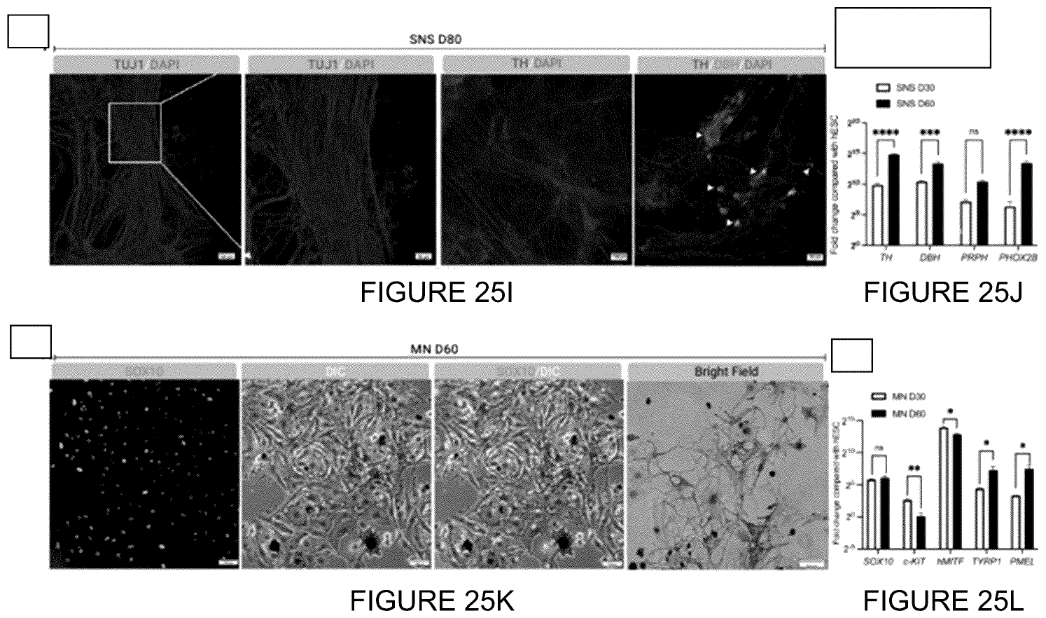

Sacral NC can be directed to diverse enteric and non-enteric NC fates. Developmental studies in the chick and mouse embryo suggest that sacral NC gives rise to the enteric nervous system as well to sympathetic and melanocytic lineages (Burns et al., Dev Biol 219, 30-43, 2000; Druckenbrod and Epstein, Dev Biol 287, 125-133, 2005: Le Douarin et al., The neural crest (Cambridge university press), 1999; Rothstein et al., Dev Biol 444 Suppl 1, S170-S180, 2018). To assess whether the sacral NC induction protocol presented here can capture differentiated lineages similar to those observed during in vivo development, conditions were established to direct hPSC-derived sacral NC towards enteric neuron, sympathetic neuron, and melanocyte lineages (FIG. 25A). For those studies, purified NC cells were isolated via sorting for CD49D, and those cells were differentiated further towards enteric neurons in the presence of glial-cell-line derived neurotrophic factor (GDNF) and Ascorbic Acid (AA). (FIG. 25A, upper row). By 7 days of differentiation (ENS D30), most sacral NC cells had lost SOX10 expression and adopted expression or early neuronal markers such as TUBB3 (immunoreactivity with TUJ-1) while maintaining HOXD13 expression. (FIG. 25B, ENS D30). By D40, most of the cells had differentiated into neurons while still retaining immature morphologies and neurite branching patterns (FIG. 25C, ENS D40). By D80, the sacral NC-derived neurons displayed more mature features with complex neuronal morphologies. The presently disclosed subject matter next characterized the various neuronal subtypes produced from sacral NC under enteric neuron differentiation conditions including Tyrosine hydroxylase (TH), GABA (γ-aminobutyric-acid-positive), nitric-oxide-synthase-positive (NOS)+, Choline acetyltransferase (ChAT), and serotonin-positive (5-hydroxytryptamine, 5-HT) neurons (FIG. 25D, ENS D80; FIG. 25H).

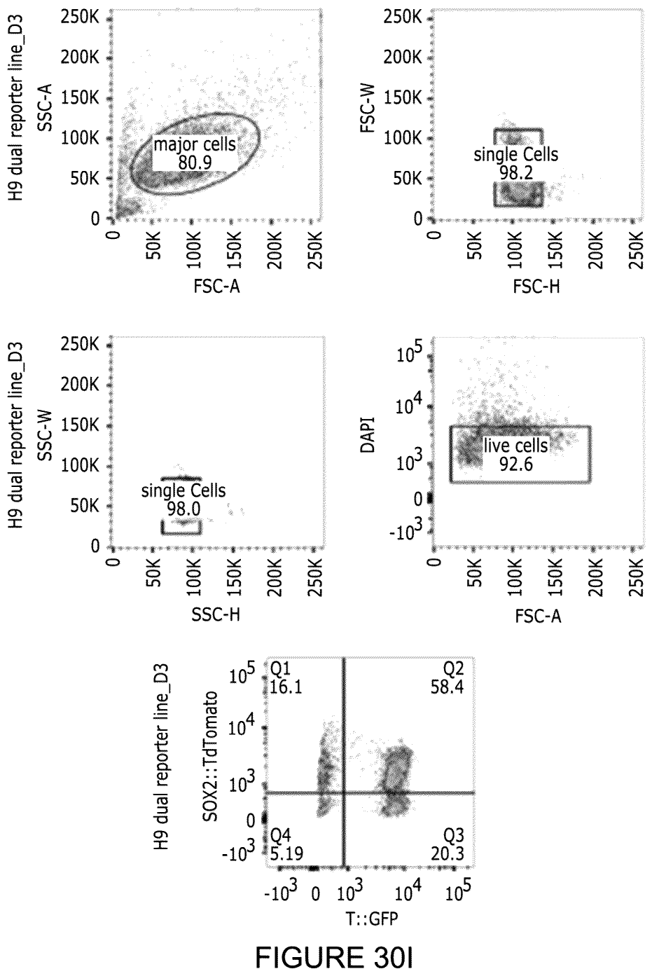

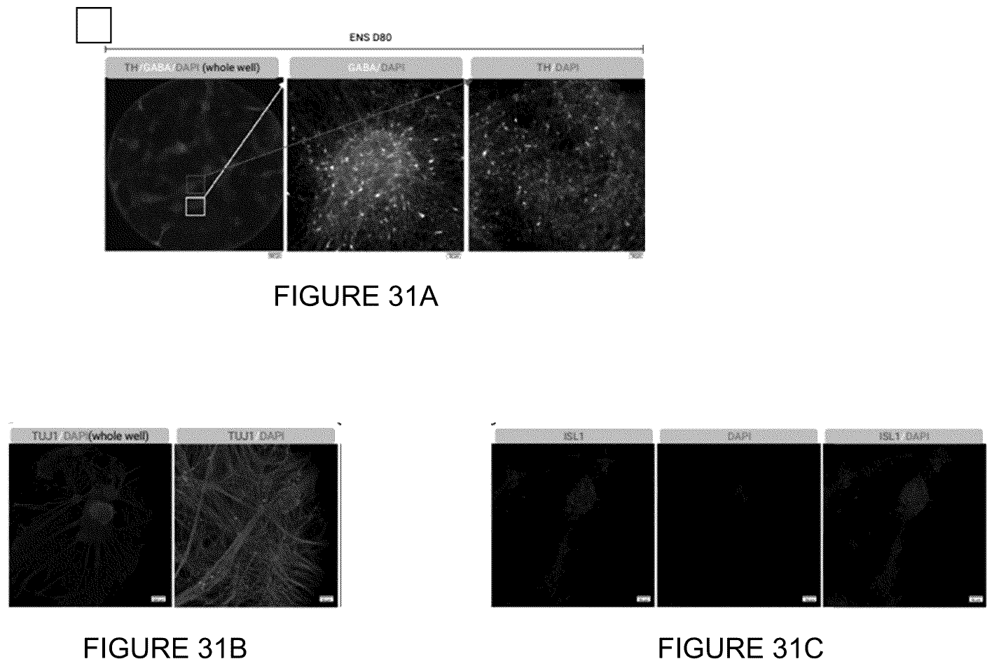

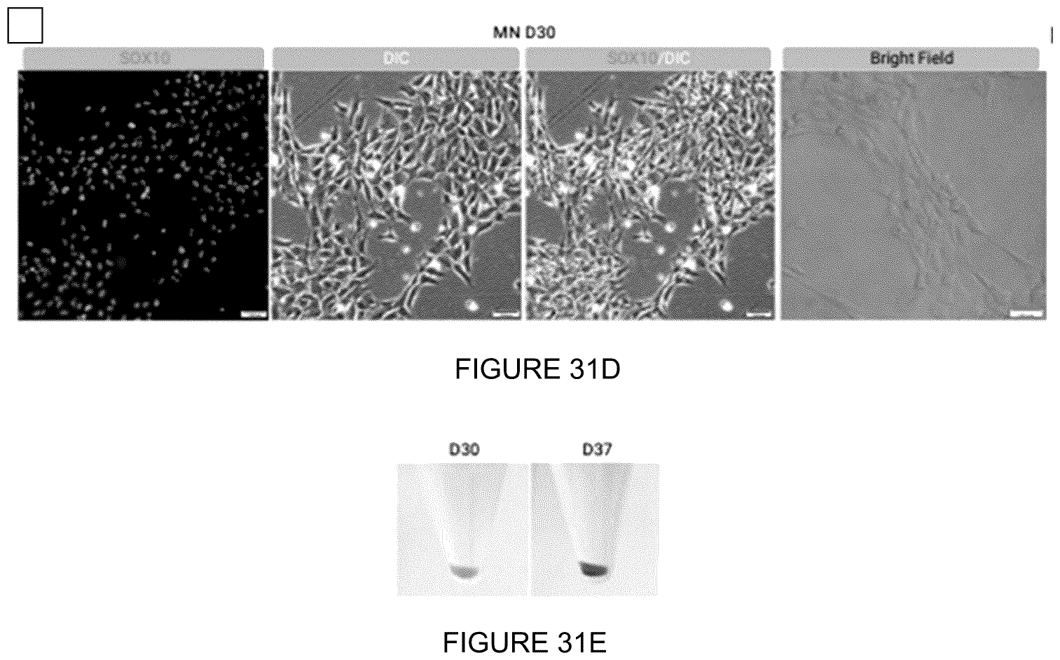

The data indicate that hPSC sacral NC cells can generate a broad diversity of subtypes characteristic of enteric neuron lineages. Gene expression data further confirmed progressive neuronal differentiation by a decrease in the expression of SOX10 (FIG. 25E) and the enteric precursor marker EDNRB (FIG. 25F). In contrast SOX2 expression was retained until D80 (FIG. 25G) suggesting the continued presence of neural or immature glial populations during enteric neuron differentiation. Interestingly, several neuron subtypes such as TH positive neurons were not randomly distributed in those cultures but showed clustering within distinct domains of the culture dish suggesting that those subtypes may be derived from temporally or spatially linked precursors populations (FIG. 31A). Such a result is compatible with Edu labeling studies in the mouse in vivo showing that distinct neuron subtypes exit the cycle at different ages (Bergner et al., J Comp Neurol 522, 514-527, 2014; Chalazonitis et al., J Comp Neurol 509, 474-492, 2008; Pham et al., Journal of comparative neurology 314, 789-798, 1991).

To direct differentiation of sacral lineage towards sympathetic neurons, CD49D⁺ purified sacral NC cells were treated with high doses of BMP4 and SHH to facilitate the differentiation into sympathetic precursors (Oh et al., Cell Stem Cell 19, 95-106, 2016). The resulting precursors were further differentiated into sympathetic neurons in the presence of GDNF, AA and Nerve growth factor (NGF) (FIG. 25A, middle row). Interestingly, sympathetic neurons displayed a distinctive morphology and formed extensive fiber bundles projecting radially (FIGS. 25I and 31B). Most neurons expressed TH and DBH compatible with sympathetic neuron identity (FIG. 25I). The presently disclosed subject matter also observed expression of ISL1 in this condition, especially in cells located along neurite bundles emanating from sympathetic neuron clusters (FIG. 31C). Sympathetic neuron identity was further confirmed by gene expression analyses of key markers (FIG. 25J).

To direct melanocytic differentiation, sacral NC cells were treated with EDN3 at the final stage of the sacral NC induction protocol to prime NC lineage towards melanoblast lineages and were purified for co-expression of p75NTR and c-Kit at D20. Further differentiation into melanocytes was achieved by using previously published differentiation protocol (Baggiolini et al., Science 373, eabc1048, 2021; Mica et al., Cell Rep 3, 1140-1152, 2013) (FIG. 25A, bottom row) which resulted in maintenance of SOX10 expression and progression via a melanoblast-like intermediate stage (FIG. 31D) to melanocytic differentiation characterized by pigment accumulation and acquisition of more mature melanocyte morphologies (FIGS. 25K and 31E). Further analysis by qRT-PCR confirmed expression of melanocytic makers including SOX10, hMITF, c-KIT and pigment related marker: TYPL1 and PMEL) (FIG. 25L). The successful derivation of sacral NC derived melanocytes may provide access to acral melanocytes, a population of melanocytes located at distal structures such as soles and palms suitable for modeling acral melanoma biology (Weiss et al., bioRxiv, 2020.2011.2014.383083, 2021).

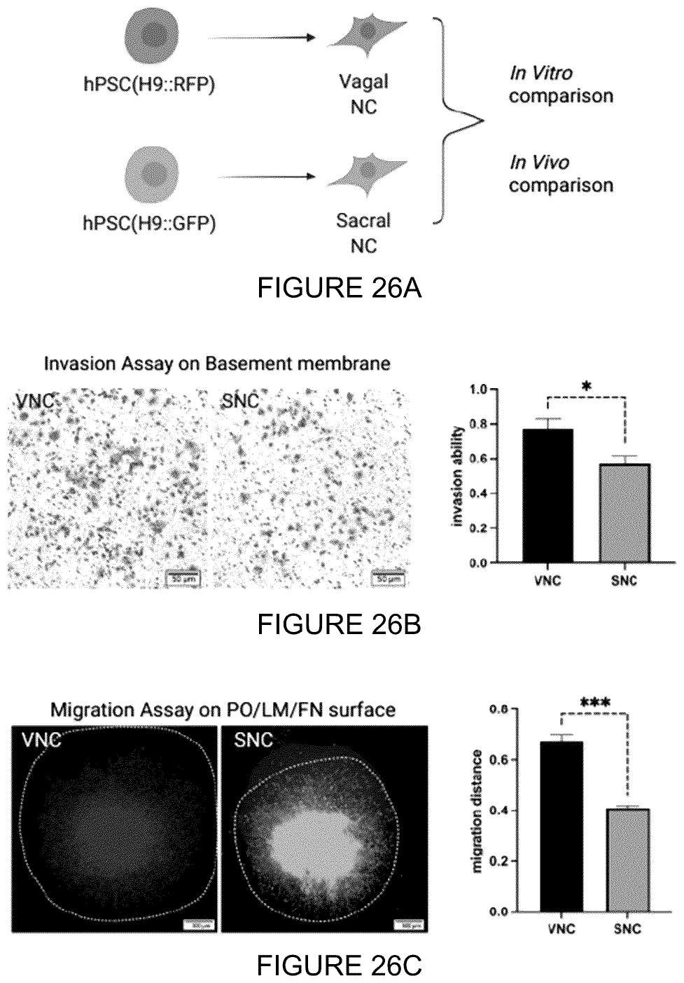

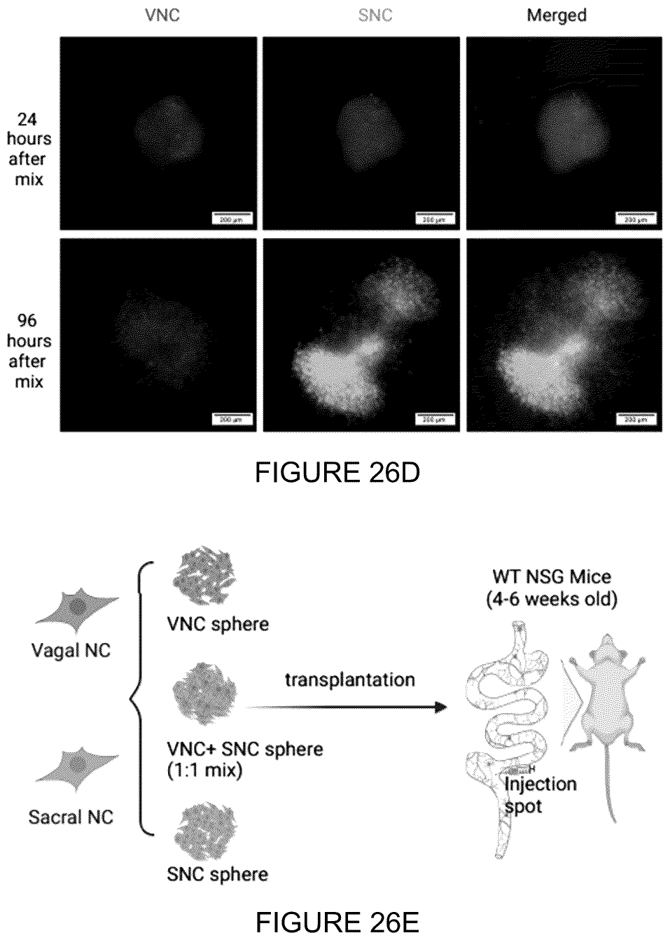

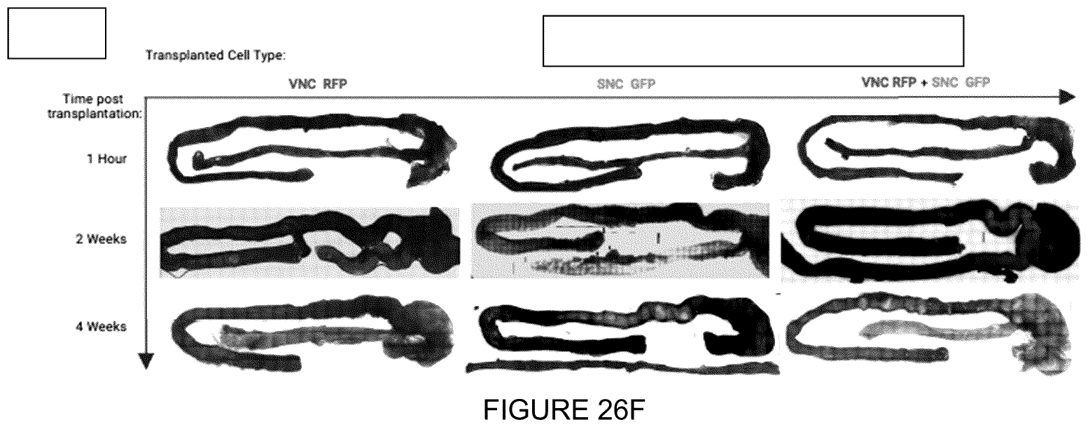

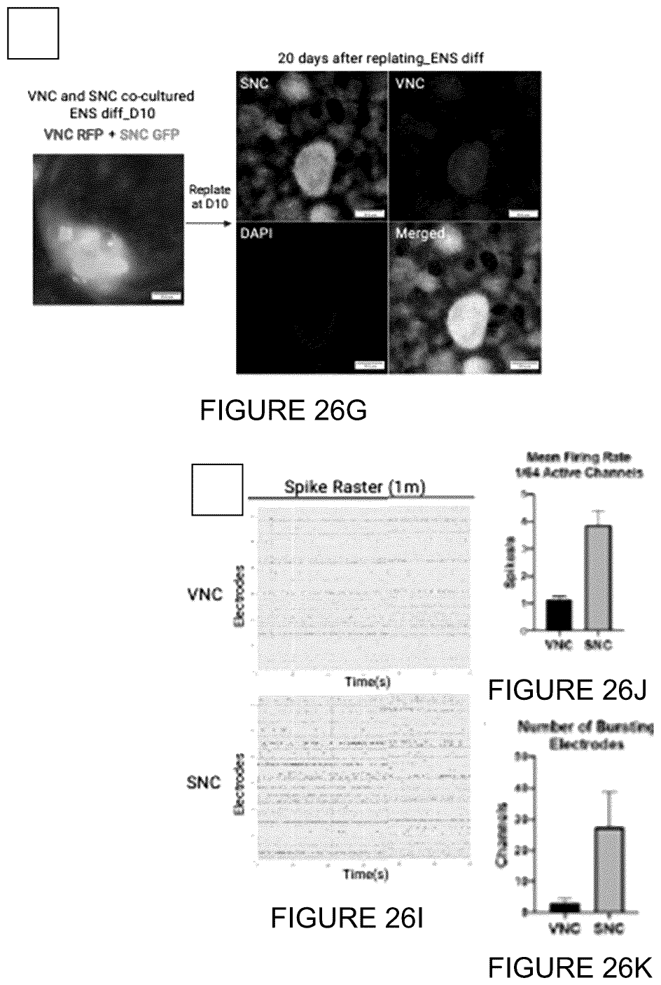

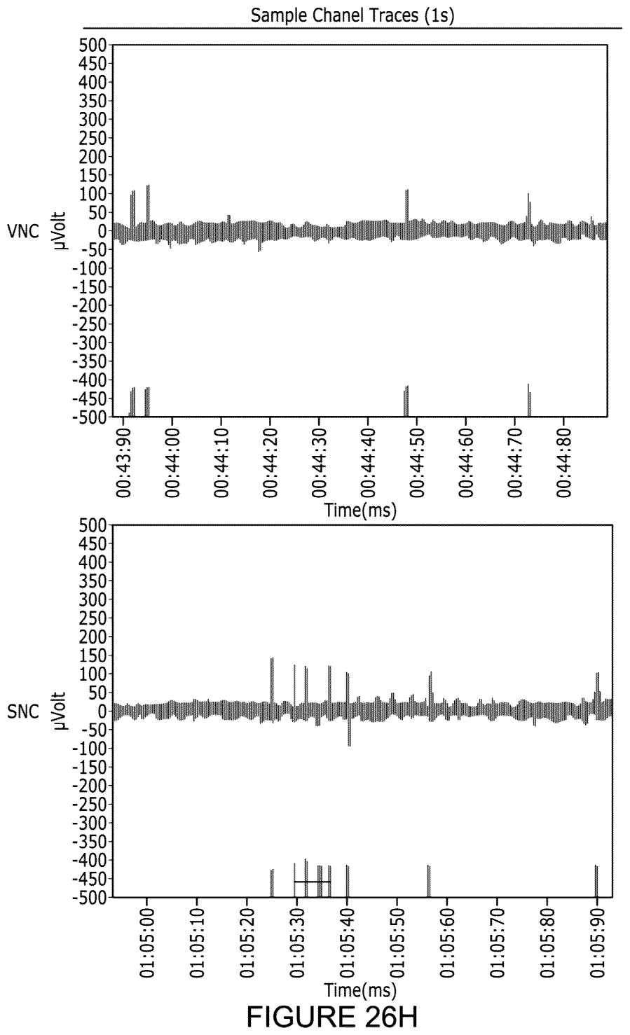

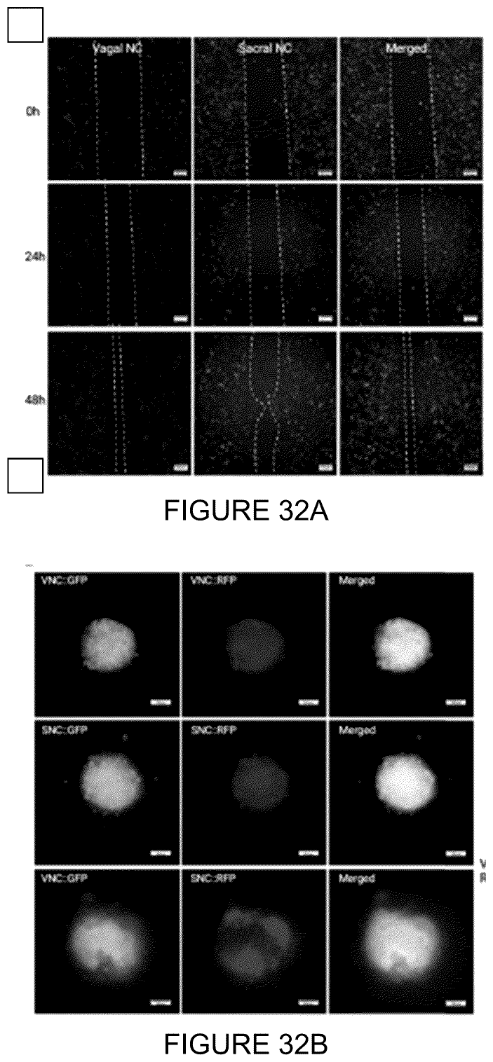

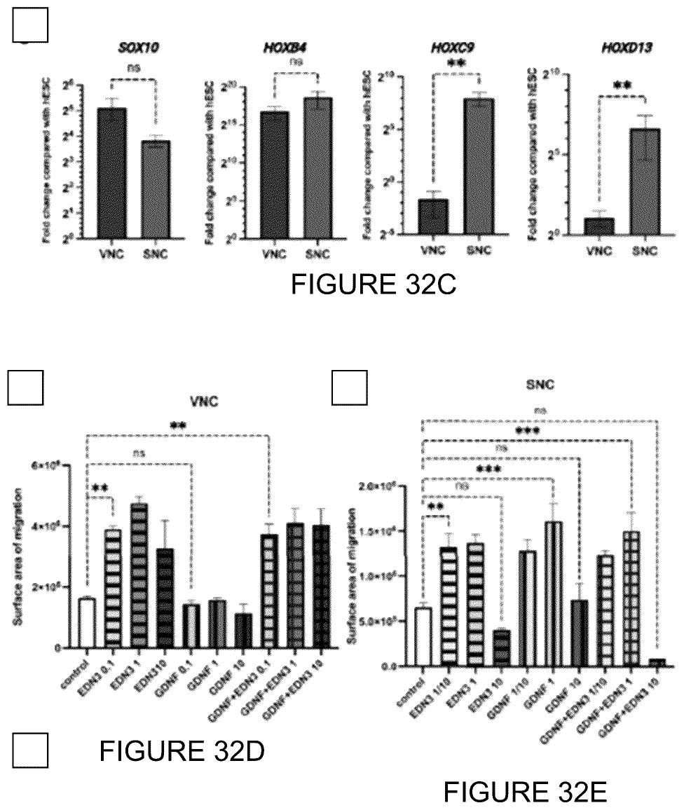

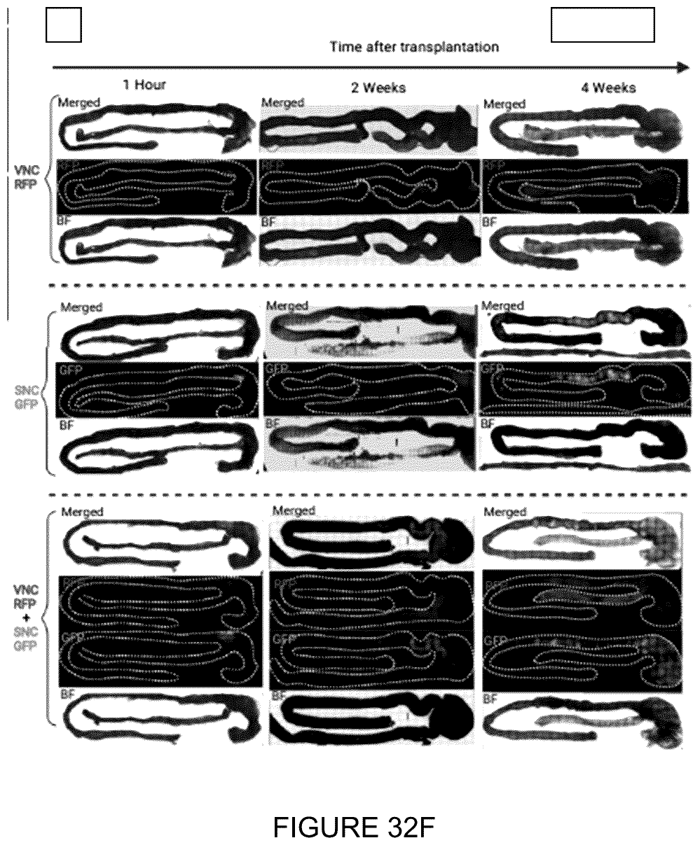

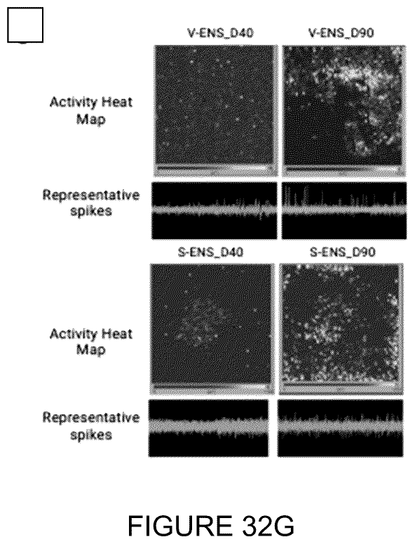

Vagal NC and Sacral NC exhibit distinct behavior both in vitro and in vivo. In quail-chick interspecies studies, it has been shown that vagal and sacral NC exhibit distinct migratory behaviors within the gut. Vagal NC cells invade the gut via the foregut and migrate in a rostrocaudal direction (Burns and Douarin. Development 125, 4335-4347, 1998; Le Douarin and Teillet, J Embryol Exp Morphol 30, 31-48, 1973). It remains unclear why vagal and sacral NC follow different migration and projection patterns. ENS lineages derived from vagal and sacral NC are closely intermingled within the gut. Therefore, it is difficult to isolate and study their specific properties in vivo, a challenge particularly pertinent for human studies. The ability to generate both sacral and vagal NC from hPSCs in vitro offers the opportunity to directly compare their behavior and other properties. To this end, vagal NC was generated from a hPSC lines with ubiquitous RFP expression and sacral NC was generated from a line expressing GFP, of which the AP identity is confirmed by expression of different NOX genes (FIG. 32C). Neural NC-derived spheroids composed of either vagal or sacral NC alone, or comprised of a 1:1 mixture of both lineages, were established followed by various functional assays in vitro and in vivo (FIGS. 26A and 26E). In trans-well invasion experiments, vagal NC showed a trend towards increased invasion compared with sacral NC (FIG. 26B). Vagal NC cells also showed an enhanced migratory capacity compared to sacral NC cells when spheroids were plated down onto PO/LM/FN (FIG. 26C), which was further confirmed in scratch assay indicating enhanced migration by 48-hour time point (FIG. 32A). An obvious co-culture phenotype was the pronounced self-sorting effect. While evenly mixed at the start of the experiment (FIG. 26D), it was noticed that vagal (Red) and sacral (Green) NC cells segregated under co-culture conditions within just a few days. The same phenotype was observed when switching cell lines to establish co-cultures of GFP⁺ vagal and RFP⁺ sacral NC. As a further control, vagal NC was generated from both GFP and RFP lines and mixed those at 1:1 ratio, which did not result in any self-sorting behavior (FIG. 32B). Those experiments point to intrinsic differences that drive the distinct behavior of vagal and sacral NC.

Even more striking was the differential in vivo behavior of vagal and sacral NC after injecting individual or mixed NC lineages into the cecum of adult NSG mice (FIG. 26E). While sacral NC migrated exclusively towards the distal colon, vagal NC spread both anterograde into the colon and retrograde into the small intestine, retrograde being the preferred route for vagal NC in the combined sacral/vagal injection studies (FIGS. 26F and 32F). Without being bound by theory, such migratory behavior may reflect a drive of the respective NC lineages to re-populate those regions of the gut targeted normally during early ENS development. Classic quail-chick grafting experiments reported that vagal NC transplanted posteriorly into the region typically colonized by the sacral NC, can migrate retrogradely in a posterior to anterior direction to populate anterior gut regions. The sacral NC grafted anteriorly into the region colonized by the vagal NC migrated anterograde into the region colonized normally by the sacral NC. Further studies will be required to determine the mechanism underlying the differential migration behavior. The presently disclosed results indicate that hPSC-derived sacral versus vagal NC show distinct responses to GDNF and EDN3 exposure, which are two of the most critical growth factors driving early NC migration during ENS development. Vagal NC migration is highly promoted by EDN3, but barely affected by GDNF (FIG. 32D). Sacral NC migration is promoted by low to medium level of EDN3 and by GDNF but suppressed at high EDN3 concentration (FIG. 32E).

Next, it was assessed whether the self-sorting behavior is dependent on cell stages. 1:1 mixed vagal and sacral NC sphere was plated down on PO/LM/FN plates, followed by ENS differentiation for 10 days. The vagal and sacral cells continued the repelling behavior (FIG. 26G, upper panel). D10 cells were dissociated and replated on PO/LM/FN again, continued with ENS differentiation for another 20 days. It was observed that the vagal and sacral derived cells were evenly mixed with no repelling behavior (FIG. 26G, lower panel), indicating the repelling effect is a NC stage specific feature. Also, it was noticed when cultured under identical, enteric neuron differentiation conditions (FIG. 25A) and maintained in direct co-culture both lineages gave rise to a diverse set of enteric neurons. However, there was a difference in the relative proportion for several of the neuronal subtypes were distinct. For example, the vagal NC cells generated a higher proportion of TH⁺ neurons and S1001B⁺ glial progenitors while sacral NC lineages were enriched in GABA and NOS neurons (data not shown). To compare the functional maturation of enteric neurons derived from either vagal versus sacral NC, recordings of electrical activity were performed using a high density multielectrode array (MEA) system containing 4096 electrodes/well. In both groups, electrical activity increased over time in concert with neuronal maturation (FIG. 32G). However, the enteric neurons derived from the sacral NC showed a significantly higher spike frequency and increased bursting events compared vagal-derived neurons at the same time point of differentiation (FIGS. 26H-26K).

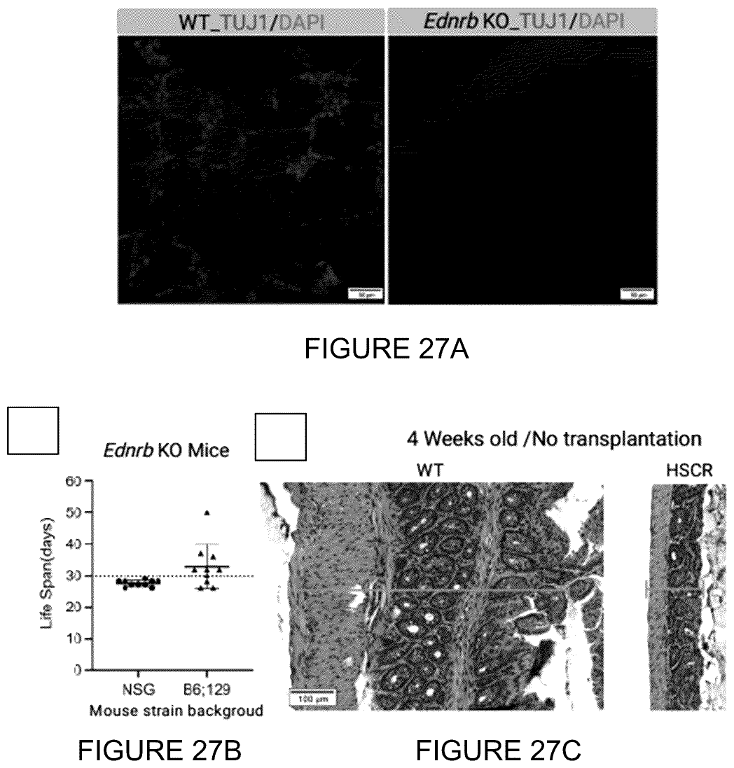

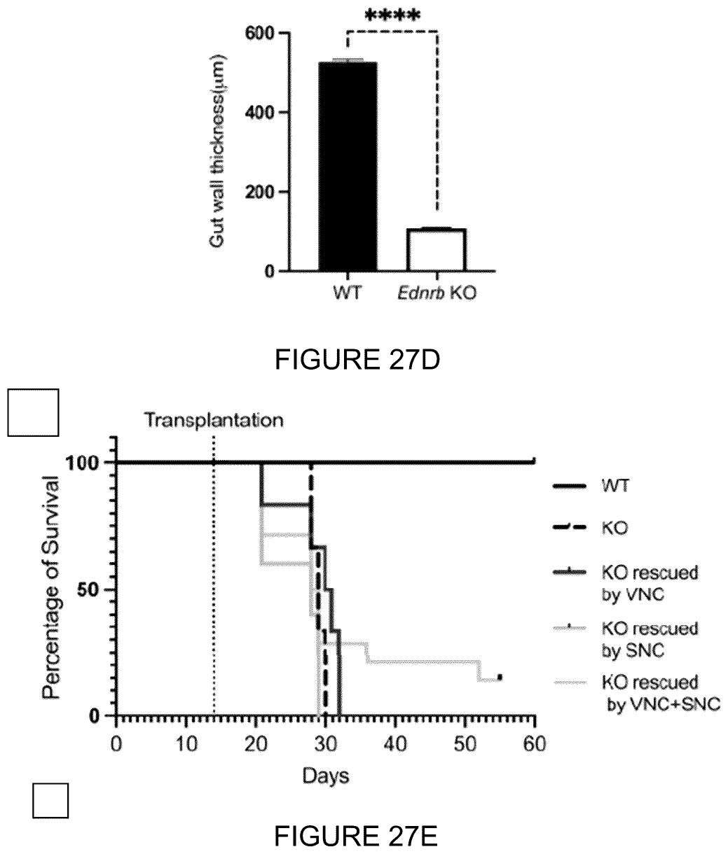

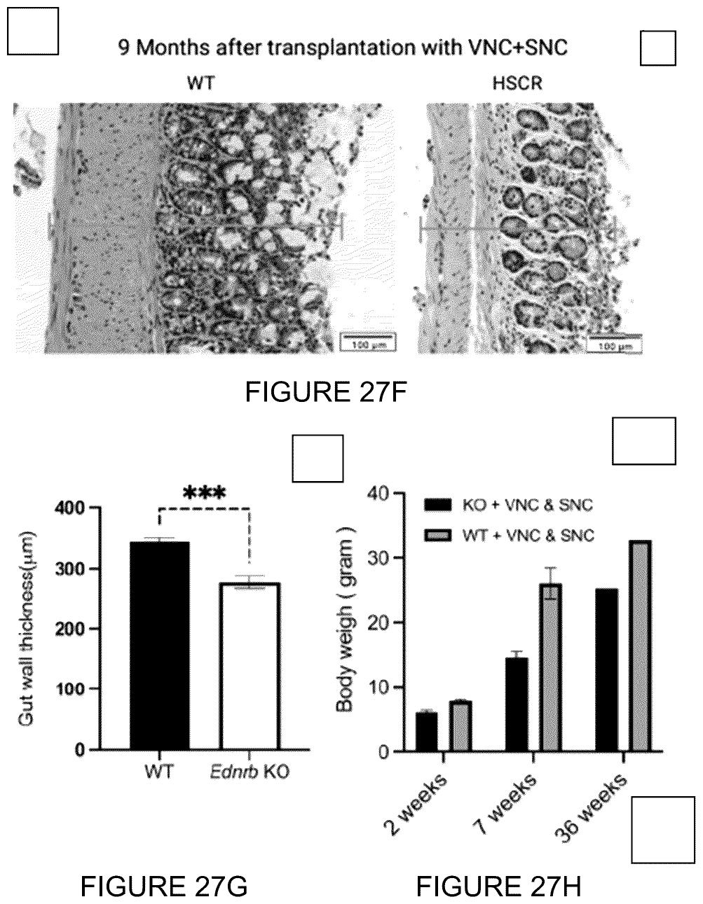

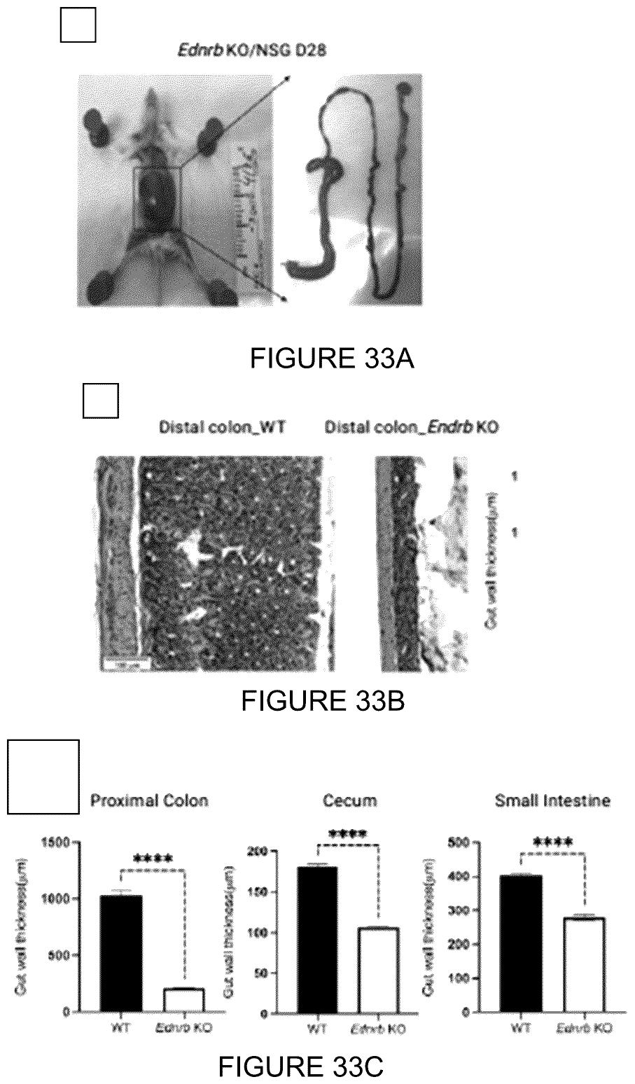

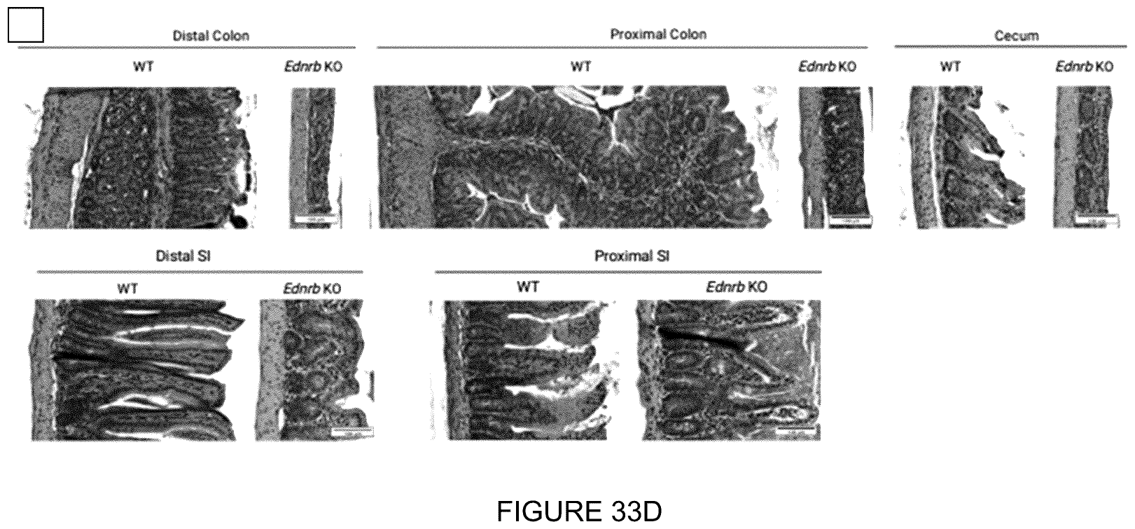

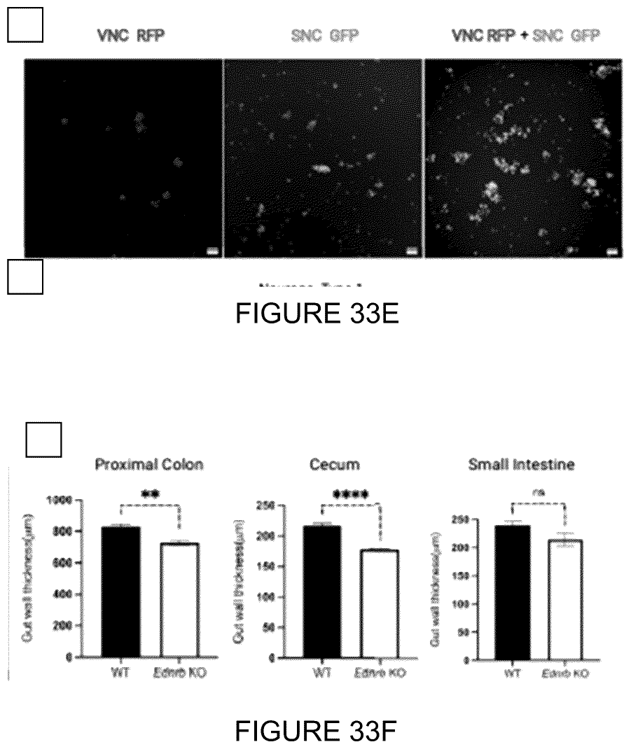

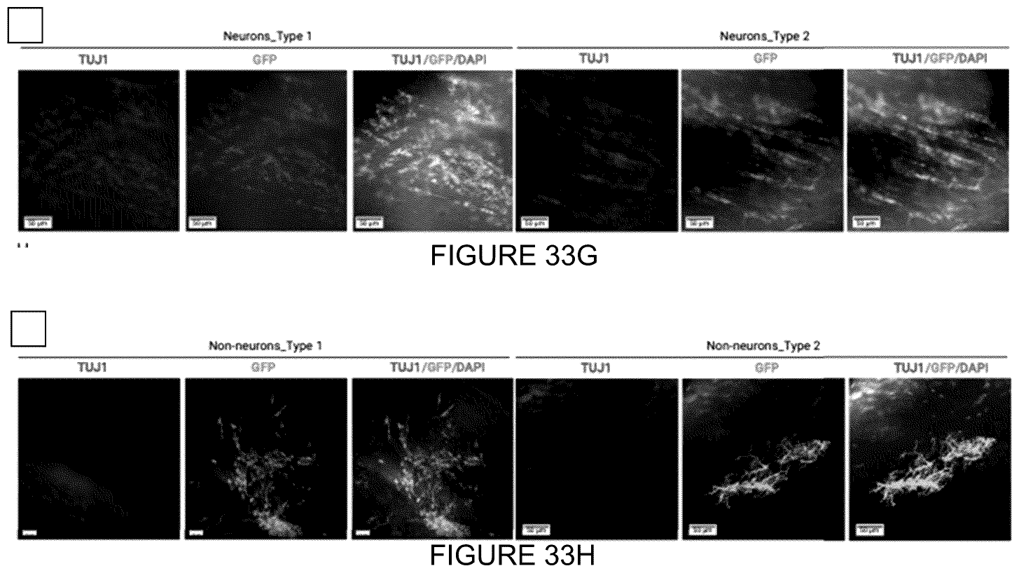

Combined vagal and sacral NC for treating HSCR mouse model of total aganglionosis. Hirschsprung disease (HSCR) is a fatal congenital disease caused by aganglionosis of the distal gut, and in some patients aganglionosis that can extend into portions of the small intestine (total aganglionosis). Mutations of the EDNRB gene are among the most common alterations causing HSCR, and mice carrying Ednrb loss-of-function mutations have been used to model HSCR disease. For the present example, the B6.129S7-E1^{um1Ywa}/FrykJ mouse was used (stock No: 021933, JAX) in which Exon 3 of the Ednrb gene is replaced by a neomycin resistance cassette. This mouse exhibits extensive aganglionosis and suffers from a megacolon phenotype. To allow for xenografting studies, the Ednrb KO strain was crossed with NSG mice (NOD.Cg-Prkdc^{scid} Il2rg^{tm1Wjl}/SzJ) for 10 generations. On the immunocompromised NSG background, Ednrb KO mice displayed a severe megacolon phenotype resulting in death of the animals by 1 months after birth (FIG. 33A). The lack of enteric neurons in KO mice was confirmed by staining with TUJ1 (FIG. 27A) and H&E staining (FIG. 33B). Interestingly, Ednrb KO mice on the NSG background showed a highly consistent disease phenotype and all the animals died between D28-D30. In contrast, Ednrb KO mice on the original 86.129S7 background showed a broader survival rate from D28-D50 (FIG. 27B). In the Ednrb KO/NSG mice, the aganglionosis extended beyond the colon all the way into the small intestine, which may explain the severity of the HSCR phenotype (FIG. 33D). Another phenotype of Ednrb KO/NSG mice is their showed much thinner gut wall and impaired villi structure compared with age matched WT NSG mice (FIGS. 27C, 27D, and 33C). Therefore, the Ednrb KO/NSG mice were used as a model of total aganglionosis and to test the potential of hPSC-derived NC lineages to rescue the most severe HSCR disease phenotypes, even more severe that those observed in the Ednrb^{sl/sl} model used in past studies (Fattahi et al., Nature 2016).

2-week-old Ednrb KO x NSG mice were injected of either vagal NC, sacral NC, a 1:1 mix of vagal and sacral NC cells or a Matrigel only control close to the cecum location (FIG. 33E). All control mice (Matrigel only) died within a month after birth and displayed a megacolon phenotype at autopsy. Mice injected with sacral NC alone failed to show any rescue effect. Among the mice injected with vagal NC, a small increase in life span was observed in some of the animals, but none of the mice survived past D40. In contrast, a subset of the mice injected with a 1:1 mixture of vagal and sacral NC showed clear improvements in survival time beyond one month with a maximal lifespan achieved of up to 9 months (FIG. 27E). While untreated HSCR mice displayed an extremely thin gut wall, this phenotype was partially rescued in the combined vagal+sacral NC grafted animals (FIGS. 27F, 27G, and 33F). Also, it was measured the body weight of the combined sacral and vagal NC injected Ednrb KO/NSG mice at around 2 weeks, 7 weeks and 36 weeks post injection. It was observed that treated mice had reduced body weight compared to their healthy siblings but continued to progressively gain weight (FIG. 27H). Finally, histological analysis of the transplanted cells in the rescued mice was performed. The presence of both vagal (RFP) and sacral (GFP) derived cells was confirmed in various sections of the gut. A higher proportion of vagal cells in the small intestine was observed (FIG. 27I, upper panel) in agreement with transplantation results in wild-type NSG mice (FIG. 26F). In contrast, sacral-derived NC cells were enriched in the distal colon (FIG. 27I, bottom panel).

The presently disclosed data demonstrate that combined vagal+sacral NC injections can rescue a very severe HSCR mouse model with aganglionosis in both the large and small intestine, a model which cannot be rescued by single spot transplantation of either vagal or sacral NC cells alone. It is possible that the self-sorting behavior of the two lineages observed in vitro (FIG. 26D) might facilitate a more rapid and widespread migration of each NC lineage in vivo. Given the major ENS defect in the small intestine, the robust re-population of the small intestine by vagal NC cells in combined grafts may mediate the improved survival of Ednrb KO/NSG mice by improving small intestine function such as food absorption or barrier function. In either case, the ability to rescue a mouse model of total aganglionosis indicates considerable therapeutic potential of combined vagal+sacral NC grafts in patients with severe HSCR.

### DISCUSSION

A human PSC-based platform for all major NC populations along AP axis. The presently disclosed subject matter offers access to the major human NC lineages along the AP axis of the body, including cranial, vagal, trunk and sacral NC. While there is evidence for considerable NC fate plasticity based on quail-chick chimera studies (Le Douarin and Kalcheim, The Neural Crest, 2 Edition (Cambridge University Press), 1999; Le Douarin et al, Development 131, 4637-4650, 2004; Le Douarin and Teillet, Developmental Biology 41, 162-184, 1974; Le Liévre and Le Douarin, J Embryol Exp Morphol 34, 125-154, 1975), previous work and the presently disclosed subject matter indicate that hPSC-derived NC cells with apparently distinct axial identities are biased in their lineage potential. The presently disclosed subject matter has established an efficient protocol to generate sacral NC. Furthermore, their differentiation potential into sacral-derived enteric neurons, sympathetic neurons and melanocytes has been demonstrated. The derivation of some NC lineages such as sympathetic or enteric neurons is restricted to specific NC domains such as trunk and sacral NC while other cell types such as melanocytes can be derived from NC cells at all AP levels (Srinivasan and Toh, Front Mol Neurosci 12, 39, 2019).

HOX gene expression and the sequential generation of NC cells. The presently disclosed subject matter reports that FGF and WNT signaling can promote the expression of posterior HOX genes without impacting NC differentiation potential (FIG. 22C). However, with increasing of FGF and WNT signaling, it was found that NC cell induction (marked by SOX10 expression) is delayed (FIG. 28I), which is reminiscent of the delay observed in the migration of sacral NC cells in vivo (Wiese et al., Developmental Biology 429, 356-369, 2017). Given the role of WNT and FGF signaling in promoting precursor cell proliferation and maintenance of stem cell-like states, prolonged periods of precursor cell identity may facilitate the sequential progression of HOX gene expression from anterior 3′ to posterior 5′ hox genes. Upon NC induction, cells inherit the respective HOX code and NC domain-specific behavior. A similar effect of FGF and WNT signaling on AP identity has been previously reported for the development of limb (ten Berge et al., Development 135, 3247-3257, 2008) and the generation of presomitic mesoderm (PSM) (Henrique et al., Development 142, 2864-2875, 2015).

Axial progenitors and the role of GDF11 in sacral level HOX gene induction. One step in deriving sacral NC was the identification of GDF11 as a factor driving the transition from trunk to sacral identity. The presently disclosed subject matter reports decreased expression of GRHL transcription factors and a sustained, negative regulation of RA signaling by transient GDF11 treatment, and the presently disclosed subject matter could also mimic, at least to a partial extent, the effect of GDF11 by inhibiting RA signaling. Studies in the mouse indicate that axial progenitor proliferation is dependent on LIN28A signaling while GDF11 controls the balance between proliferation and differentiation via regulating HOX3 genes (Aires et al., Developmental Cell 48, 383-395.e388, 2019). Another study shows that LIN28A/let-7 pathway regulates HOX code expression via regulation of polycomb-related genes (Sato et al., Elife 9, e53608, 2020). Both studies reported an inhibitory relationship between LRN28A and HOX13 expression similar to that observed in our current data set (data not shown).

The data of the present example indicate that SOX2/Brachyury(T) double positive NMPs can give rise to posterior NC lineages including trunk and sacral NC. NMPs have been shown to contribute to the spinal cord and paraxial mesoderm in vitro (Denham et al., Stem cells 33, 1759-1770, 2015; Gouti et al., PLoS biology 12, 2014; Lippmann et al., Stem Cell Reports 4, 632-644, 2015; Tsakiridis and Wilson, F1000 Research 4, 2015) and in vivo (Brown and Storey, Current Biology 10, 869-872, 2000; Cambray and Wilson. Two distinct sources for a population of maturing axial progenitors, 2007; Gouti et al., PLoS biology 12, 2014; Henrique et al., Development 142, 2864-2875, 2015; Iimura and Pourquié, Nature 442, 568-571, 2006; Olivera-Martinez et al., PLoS biology 10, e1001415, 2012; Tzouanacou et al., Developmental cell 17, 365-376, 2009; von Kölliker, Die embryonalen Keimblätter und die Gewebe (Wilhelm Engelmann), 1884). The contribution of hPSC-derived NMPs to NC development has been previously reported for the trunk NC lineage (Frith et al., Elife 7.10.7554/eLife.35786, 2018). In fact, trunk NC-derived lineages, such as sympathetic neurons, emerge readily in protocols that involve an NMP intermediate as compared to studies attempting to induce trunk NC via RA and BMP mediated patterning of cranial NC lineages (Huang et al., Sci Rep 6, 19727, 2016; Oh et al., Cell Stem Cell 19, 95-106, 2016). A key unresolved question is whether these in vitro data reflect the in vivo requirement of an NMP-intermediate during trunk NC development. A recent study in the zebrafish integrated single cell epigenomics and transcriptomics data of NMPs and NC cells. Those data indicate that at least a portion of posterior NC in the developing zebrafish embryos is derived from NMPs (Martyna Lukoseviciute, Biorxiv. https://doi.org/10.1101/2021.02.10.430513, 2021).

Difference between vagal and sacral NC-derived enteric neural lineages. The direct comparison of vagal and sacral NC lineages revealed differences in migratory behavior, relative proportion of neuronal and glial subtypes and in neuronal activity. Access to defined neuronal subtypes will be relevant for applications in disease modeling and regenerative medicine beyond HSCR, such as the use of NOS⁺ neurons for the potential treatment of diabetic neuropathy. The presently disclosed subject matter observed a striking repellent action and cell sorting behavior between sacral and vagal NC, in addition to their distinct responses to GDNF and EDN3. There is evidence for a specific role of cadherins, such as CDH19, in sacral NC migration (Huang et al., Gastroenterology 162, 179-192.e111, 2022), and differences in cadherins or integrins may mediate some of the repellent interactions. Interestingly, the differences were only observed at the NC precursor stage while vagal and sacral NC-derived neurons, mixed together at the neuronal differentiation stage, didn't show similar effect (FIG. 26G).

A role for sacral NC in stem cell-based cell therapy for HSCR. Over the last several decades, there has been considerable interest in developing cell-based therapies for the treatment of HSCR and related ENS disorders (Burns et al., Dev Biol 417, 229-251, 2016). Previous work (Fattahi et al., Nature 531, 105-109, 2016) has provided proof of concept for the use of human PSC-derived NC in preclinical models of the disease. One advantage of hPSC-derived versus primary ENS precursors (or other alternative cell sources) is the scalability and access to the earliest stages of ENS development, when cells are capable of extensive in vivo migration (Zhang et al., Dev Biol 446, 34-42, 2019). There have been doubts about whether transplanted NC cells can differentiate properly in vivo and manage to rescue the missing neuronal function. The presently disclosed subject matter has data to show that transplanted sacral NC can spontaneously differentiate into neuronal cells, which can project towards the villi (FIG. 33G, left panel), or along the longitude muscle lever (FIG. 33G, right panel), and non-neuronal cells (FIG. 33H), of which some are perhaps glia-like cells based on the morphology.

In the present example, it is shown that the combination of vagal and sacral NC can rescue a severe model of HSCR affecting both the large and small intestine. The consistency of the disease phenotype in the Ednrb KO/NSG mice enabled to confidently detect graft-mediated effects on animal survival. On the other hand, the severity of the phenotype made the surgery challenging with only a very short time window for the grafted cells to achieve a rescue. Why the combined grafting strategy is required to rescue such a severe mouse model, while single vagal NC transplantation was sufficient to rescue the less severe Ednrb^{sl/sl} model (Fattahi et al., Nature 531, 105-109, 2016), can be the topic of future studies. One possibility is that the aganglionosis in the small intestine is responsible for the highly consistent survival phenotype by preventing absorption of nutrients and other factors. This hypothesis is supported by the dramatic difference in weight between Ednrb KO/NSG versus WT NSG animals and it would imply that the rescue of small intestine function via enhanced vagal NC migration in combined grafts is critical to achieve survival. At the level of tissue organization, the presently disclosed subject matter observed a partial rescue of thickness of the gut epithelium in animals injected with combined grafts which in turn could contribute to improved food absorption and barrier function in addition to any potential impact on gut motility. The increased number of vagal NC-derived cells within the small intestine upon the combined grafting strategy may be related to their mutually repellent interactions at transplantation to enhance migration of vagal cells into the small intestine. If the main driver for rescue of animals in the combined grafting group is their enhanced migration and repopulation of the small intestine, it is conceivable that this limitation could be overcome in humans by performing multiple injections along the small intestine, a strategy not applicable to the much smaller mouse gut. On the other hand, if there are unique differences in the functional properties of the differentiated cell types derived from sacral versus vagal NC, it will be important to include both populations in future translational efforts and to administer a combined cell product in HSCR patients with total aganglionosis.

### Methods

Experimental Model and Subject Details. Experiments were largely carried out using the human embryonic stem cell (hESC) line H9 (WA-09) or reporter lines derived from H9 (WA-09). The human embryonic stem cell (hESC) lines H1 (WA-01), HUES6 and MEL1 were also used to repeat the experimental results generated with H9. Human induced pluripotent stem cell (iPSCs) lines 348.7, 706.3 and 864.3 (previously published in Cederquist et al., Nature biotechnology 37, 436-444, 2019; Cornacchia et al., Cell stem cell 25, 120-136, 2019) were also used to repeat critical experiments. Reporter lines used in this study are as follows. The H9-derived SOX10::GFP reporter line was generated as reported previously (Chambers et al., Nature biotechnology 30, 715, 2012). The H9-derived GFP and mCherry lines were generated by lentiviral infection. The H9 and H1-derived SOX2::Tomato/T::GFP dual reporter lines were generated through a CRISPR knock-in method, which was validated by PCR and sequencing. All cell lines used were karyotypically normal as assessed by G-banded chromosomal analysis. All modified cell lines used were generated by the MSKCC Stem Cell Core as described in the methods section below. Mouse strains were NSG (NOD.Cg-Prkdcscid Il2rgtm1Wjl/SzJ) mice and an NSG derived disease model line carrying the Ednrb^{tm1Ywa}/FrykJ mutation (Frykman et al., 2015; Hosoda et al., 1994).

Culture of hESC and iPSC in Essential (E8) medium. hESC or iPSC were plated on 10 cm dishes coated with Vitronectin (1:100 diluted in DPBS and coated in cold room overnight) and maintained in E8 medium (Thermo Fisher, A1517001). The E8 medium was changed every day and the cells were passaged every 3-5 days at 70-85% confluence. The cells were passaged using EDTA dissociation (0.5 mM EDTA+1.8 g/l (30.8 mM) NaCl (Sigma-Aldrich) in DPBS) at 1:15 ratio as described previously.

NC differentiation. The basic NC differentiation protocols used are based on previous works (Fattahi et al., Nature 531, 105-109, 2016; Tchieu et al., Cell Stem Cell 21, 399-410 e397, 2017). Briefly, matrigel (Thermo Fisher, A1413201) was diluted at 1:100 ratio in DMEMF-12 (Thermo Fisher, 21331020) and plates were coated with the diluted matrigel overnight at 4° C. For inducing differentiation, hESCs or iPSCs were dissociated at 70%-80% confluence using EDTA dissociation buffer and replated as a single cell suspension on matrigel-coated 24-well plates at a density of 100K cells/cm² in E6 medium containing 10 μM ROCK-inhibitor (Y-27632; R&D, 1254). The cells were kept in E6 medium (Thermo Fisher, A1516401)+Y-27632 overnight. The next day, the medium was switched to NC induction medium. For cranial differentiation, the cells were kept in E6 medium containing 10 μM SB431542 (R&D, 1614), 1 ng/BMP4 (R&D, 314-BP), and 0.6 μM CHIR (R&D, 4423) for the first 2 days and then switched to E6 medium containing 10 μM SB431542 and 1.5 μM CHIR for 10 days. For vagal NC the same conditions were used with an additional 1 μM RA (Sigma, R2625) added to the medium starting from D6. To establish the sacral NC protocol, the cranial NC protocol was modified with varying concentrations of FGF2 (R&D, 233-FB/CF), CHIR and GDF11 (Peprotech, 120-11), added at different time points as detailed in the results section. For the generation of melanocytes, 100 nM EDN3 (American Peptide company, 88-5-10B) was added to the sacral NC protocol from day 14 to day 20. In experiments testing the RA hypothesis, the sacral NC protocol was modified using either 1 μM RA (Sigma, R2625) or 100 nM AGN (Tocris, 5758) as detailed in the experimental design section.

RNA extraction and RT-qPCR. RNA was prepared from samples collected with the Zymo Direct-zol Kit and extracted using the Direct-zol RNA MiniPrep kit. cDNA was generated using the iScript Reverse Transcription Supermix for RT-qPCR. For qPCR analysis, primers were obtained from QIAGEN or IDT and the reactions were performed following manufacturers' instructions using SsoFast EvaGreen® Supermix. The Assays were run on BioRad CFX384 Real-Time PCR machine. Results were normalized to GAPDH housekeeping genes.

Immunostaining for cultured cells. Cultured cells were fixed with 4% PFA for 10-20 minutes, then blocked and permeabilized in IF buffer (PBS+1% BSA+0.3% Triton X-100). Primary antibodies were diluted according to the manufacturer's recommendation in IF buffer+5% normal donkey serum or 5% normal goat serum. The fixed cells were incubated with primary antibody overnight at 4° C. Primary antibody was washed with PBS+0.01% Tween-20 (PBS-T) for 10 minutes, repeated 3 times. Cells were then incubated in secondary antibody conjugated with Alexa Fluor 488-555-, or 647-diluted at 1:500 in IF buffer+5% normal donkey serum or 5% normal goat serum for 1 hour at room temperature. The secondary antibody was also washed with PBS-T 3 times, 10 minutes each time. Between the second wash and third wash, cells were stained with 4′,6-diamidino-2-phenylindole (DAP1) for 10 minutes.

Flow cytometry. Cells were disassociated with Accutase for 20-40 minutes depending on cell types at 37° C. After neutralizing with DMEMF-12 containing 2% FBS and washing once with DMEMF-12, cells were filtered with 40 μm filter. If the cells were derived with reporter lines, the cells were resuspended in DMEMF-12 containing 2% FBS and 10 μM DAPI and sorted or analyzed directly by BD LSRFortessa™ or BD FACSAria™. If the cells needed to stain for cell surface markers, the cells were resuspended in DMEMF-12 containing 2% FBS and diluted primary antibodies following manufacturers' instruction and incubated on ice for 30 minutes. The cells were washed with DMEMF-12 twice and then resuspended in DMEMF-12 containing 2% FBS and 10 μM DAPI for sorting or analysis. If the cells needed to be stained for intracellular markers, the cells were resuspended 1×PBS with 2 μg propidium iodide to determine the live or dead population. Then the cells were washed with PBS, then fixed and permeabilized using BD Cytofix/Cytoperm (BD Bioscience, 554722) on ice for 30 min. Fixed cells were then permeabilized and stained using 1×BD Perm/Wash Buffer (BD Bioscience, 554723) following the manufacturer's instructions. Results were analyzed using FlowJo.

RNA sequencing (RNA-seq). Cells were disassociated with Accutase for 20 minutes at 37° C. The cells were collected into 15 mL falcon tubes and DMEMF-12 containing 2% FBS was added to neutralize the enzyme. The cells were spun down and washed with PBS twice. The cells were spun down again in 1.5 mL Eppendorf tube and resuspended in 500 μL TRIzol (ThermoFisher catalog #15596018) and stored at −80° C. When all data points were collected, the samples underwent RNA extraction and RNA-seq as performed by the IGO core at MSKCC. Briefly, phase separation in cells lysed in 1 mL TRIzol Reagent was induced with 200 μL chloroform and RNA was extracted from the aqueous phase using the miRNeasy Mini Kit (Qiagen catalog #217004) on the QIAcube Connect (Qiagen) according to the manufacturer's protocol with 350 μL input. Samples were eluted in 15 μL RNase-free water. After RiboGreen quantification and quality control by Agilent BioAnalyzer, 500 ng of total RNA with RIN values of 9.6-10 underwent polyA selection and TruSeq library preparation according to instructions provided by Illumina (TruSeq Stranded mRNA LT Kit, catalog #RS-122-2102), with 8 cycles of PCR. Samples were barcoded and run on a HiSeq 4000 in a PE100 run, using the HiSeq 3000/4000 SBS Kit (Illumina). An average of 53 million paired reads was generated per sample and the percent of mRNA bases averaged 85%.

RNA-seq bioinformatics. STAR aligner (Dobin et al., Bioinformatics 29, 15-21, 2013) was used to map reads to the human genome (GRCh37). The 2-pass mapping method (Engström et al., Nature methods 10, 1185-1191, 2013) was used, in which the reads are mapped twice. SAM files were processed and converted to BAM format using PICARD tools and then HTseq was used to compute the expression count matrix from the mapped reads. DESeq2 (Love et al., Genome Biology 15, 550, 2014) was used to normalize the raw counts (Median of Ratios method) and perform differential gene expression analysis. Data are presented on a gene-by-gene basis as normalized counts or after carrying out a variance-stabilizing transformation followed by PCA analysis.

ATAC sequencing (ATAC-seq). Cells were disassociated with Accutase for 20 minutes at 37° C. The cells were collected into 15 mL falcon tubes and DMEMF-12 containing 2% FBS was added to neutralize the enzyme. The cells were spun down and washed with PBS twice. The cells were spun again in a 1.5 mL Eppendorf tube and resuspended in 500 μL stem cell banker cell freezing buffer and stored at liquid nitrogen tank. When all data points were collected, the samples were sent to IGO core. ATAC-seq was performed by IGO core at MSKCC. Profiling of chromatin was performed by ATAC-Seq as described in Buenrostro et al., Nat Methods 10, 1213-1218, 2013. Briefly, 50,000 viably frozen neural crest cells were washed in cold PBS and lysed. The transposition reaction was carried out using TDEI Tagment DNA Enzyme (Illumina catalog #20034198) incubated at 37° C. for 30 minutes. The DNA was cleaned with the MinElute PCR Purification Kit (QIAGEN catalog #28004) and material was amplified for 5 cycles using NEBNext High-Fidelity 2×PCR Master Mix (New England Biolabs catalog #M0541L). After evaluation by real-time PCR, 7-10 additional PCR cycles were done. The final product was cleaned by aMPure XP beads (Beckman Coulter catalog #A63882) at a 1× ratio, and size selection was performed at a 0.5× ratio. Libraries were sequenced on a HiSeq 4000 in a PE100 run, using the HiSeq 3000/4000 SBS Kit (Illumina). An average of 42 million paired reads were generated per sample. Transposition of native chromatin for fast and sensitive epigenomic profiling of open chromatin, DNA-binding proteins, and nucleosome position.

ATAC-seq bioinformatics. ATAC-seq data was processed following the recommendations of the ENCODE consortium (The ENCODE Consortium ATAC-seq Data Standards and Prototype Processing Pipeline https://www.encodeproject.org/atac-seq/). Reads were aligned to the human reference genome (GRCh37) with BWA-backtrack (Li and Durbin, Bioinformatics 25, 1754-1760, 2009). Post-alignment filtering was done with samtools (Li et al., Bioinformatics (Oxford, England) 25, 2078-2079, 2009) and Picard tools (Institute) to remove unmapped reads, improperly paired reads, non-unique reads, and duplicates. To identify regions of open chromatin represented by enrichments of reads, peaks were called with MACS2 (Liu, Methods Mol Biol 1150, 81-95, 2014). Peaks with an adjusted P<0.01 were merged, quantified, and normalized using DiffBind v3.2.1 (Brown, DiffBind: Differential binding analysis of ChIPSeq peak data, 2022). ATAC-seq signal profiles were created with bamCoverage from the deepTools suite (Ramirez et al., Nucleic Acids Res 44, W160-165, 2016) using the following parameters: —bs 10—normalizeUsing RPGC—effectiveGenomeSize 2776919808—blackListFileName hg19—blacklist.v2.bed—ignoreForNormalization chrX chrY—ignoreDuplicates—minFragmentLength 40. Blacklisted regions (Amemiya et al., Sci Rep 9, 9354-9354, 2019) were retrieved from https://sites.google.com/site/anshulkundaje/projects/blacklists.

Generation and validation of H9 SOX2::tdTomato/T::GFP dual reporter line. H9 SOX2::tdTomato/T::GFP dual reporter lines were generated using CRISPR-Cas9 based HDR method (Zhong et al., Protocol for the Generation of Human Pluripotent Reporter Cell Lines Using CRISPR/Cas9. STAR Protoc 1, 2020). Briefly, the sgRNA were designed to target a sequence close to the stop codon of the SOX2 or T gene. Each target sequence was cloned into the pX330-U6-Chimeric_BB-CBh-hSpCas9 vector (Addgene plasmid #42230) to make the gene targeting constructs. For SOX2 targeting, a donor plasmid containing a 400 bp left homology arm, followed by a P2A-H2B-tdTomato cassette, and a 400 bp right homology arm was used as the template for HDR. The sgRNA and the donor plasmid were electroporated into H9 cells using a Lonza 4D-Nucleofector instrument with Solution “Primary Cell P3”, and Pulse Code “CB-150”. 4 days after electroporation, tdTomato⁺ cells were sorted out and expanded. Single-cell clones were then isolated, and the following PCR and Sanger sequencing were used to verify knock-in. The T::GFP reporter cells were generated on top of the validated SOX2::tdTomato cells. For T targeting, a donor plasmid containing a 400 bp left homology arm, followed by a P2A-12B-GFP cassette, a floxed puromycin selection cassette (loxP-PGK-puro-loxP) and a 400 bp right homology arm was used as the template for HDR. The sgRNA and the donor plasmid were electroporated in to H9 SOX2::tdTomato cells. 0.5 μg/mL Puromycin was added to the 3 days post-electroporation for 4 days. Single-cell clones were generated, PCR and Sanger-sequencing were used to identify correctly knock-in clones. The dual reporter cells expressed SOX2-tdTamato at the hESC stage, and expressed T::GFP in hESC-derived mesendoderm stage, which performed using a 1-day mesendoderm differentiation protocol (Zhong et al., Protocol for the Generation of Human Pluripotent Reporter Cell Lines Using CRISPR-Cas9, STAR Protoc 1, 2020). The dual reporter showed a normal karyotyping (G-banding).

The sg RNA and primers used were as followings:

| | |
|---|---|
| SOX2-sgRNA target | CCTCTCACACATGTGAGGGC |
| | (SEQ ID NO: 1) |
| T-sgRNA target | ACCTTCCATGTGAAGCAGCA |
| | (SEQ ID NO: 2) |
| SOX2-PCR-F1 | TACCTCTTCCTCCCACTCCA |
| | (SEQ ID NO: 3) |
| STCLM0221R | CCACAGAGATGGTTCGCCAGT |
| | (SEQ ID NO: 4) |
| T-PCR-F1 | CTACACACCCCTCACCCATC |
| | (SEQ ID NO: 5) |
| 153R | CTAAAACCTGCTGTCCTCAACTATG |
| | (SEQ ID NO: 6) |

Generation of lentivirus based H9::mCherry and H9::GFP cyto-reporter line. The Lentiviral vectors: PLVX-EF1a-mcherry and PLVX-EF1a-GFP were purchased from Takara. The Lenti-virus were made in HEK293T cells with packaging plasmid: psPAX (Addgene: 12260), and envelope plasmid pMD2.G (Addgene 12259). H9 cells were infected with the lentivirus. The mCherry or GFP expression cells were sorted at day 4 post-infection and expanded. Fluorescent images showed the H9::mCherry or H9::GFP constitutively express mCherry or GFP, respectively. The cells maintained as normal karyotype.

Enteric neuron differentiation. In vitro differentiation of NC to enteric neurons was carried out as previously described in (Fattahi et al., Nature 531, 105-109, 2016). Briefly, the vagal NC or sacral NC cells were purified by FACS by the cell surface marker CD49D. The purified NC were then cultured in neural spheroid medium for 4 days in ultra-low attachment plates. Neural spheroid medium is comprised of neurobasal (NB) medium supplemented with 1-glutamine (Gibco, 25030-164), N2 (Stem Cell Technologies, 07156), B27 (Life Technologies, 17504044) and NEAA, CHIR99021 (3 μM, Tocris Bioscience, 4423) and FGF2 (10 nM, R&D Systems, 233-FB-001MG/CF). The aggregated NC spheroids were plated on poly-ornithine/laminin/fibronectin (PO/LM/FN)-coated plates. The method PO/LM/FN coated plates preparation has be previously described in Zeltner el al. (2014). Then the cells were switched to enteric neuron differentiation medium, containing neurobasal (NB) medium supplemented with 1-glutamine (Gibco, 25030-164). N2 (Stem Cell Technologies, 07156), B27 (Life Technologies, 17504044) and NEAA, containing GDNF (25 ng/mL−1, Peprotech, 450-10) and ascorbic acid (200 μM, Sigma, 4034-100 g). The cells were fixed for immunostaining or collected for gene expression analysis at different days of differentiation.

Sympathetic neuron dilferentiation. In vitro differentiation of NC to sympathetic neurons was carried out as described in (Frith et al., Elife 7.10.7554/eLife.35786, 2018). Briefly, the sacral NC cells were purified using FACS based on expression of the cell surface marker CD49D. The purified NC were then cultured in neural spheroid medium for 4 days in ultra-low attachment plates. The aggregated NC spheroids were plated on poly-omithine/laminin/fibronectin (PO/LM/FN)-coated plates. The cells were then cultured in a medium containing high BMP4 and SHH for 4 days. This medium consisted of neurobasal (NB) medium supplemented with 1-glutamine (Gibco, 25030-164), N2 (Stem Cell Technologies, 07156), B27 (Life Technologies, 17504044), NEAA, BMP4 (50 ng/mL, R&D, 314-BP) and recombinant SHH (C25II) (50 ng/mL, R&D, 464-SH). Following this, the cells were switched to sympathetic neuron differentiation medium, containing neurobasal (NB) medium supplemented with 1-glutamine (Gibco, 25030-164), N2 (Stem Cell Technologies, 07156), B27 (Life Technologies, 17504044). NEAA, ascorbic acid (200 μM, Sigma, 4034-100 g), NGF (10 ng/mL, Peprotech, 450-01), BDNF (10 ng/mL, R&D, 248-BDB) and GDNF (10 ng/mL, Peprotech, 450-10). The cells were fixed for immunostaining or collected for gene expression analysis at different days of differentiation.

Melanocyte differentiation. EDN3 (100 nM, American Peptide company, 88-5-10B) and BMP4 (5 ng/ml, R&D, 314-BP) was added from day 14 to day 20 on top of the sacral neural crest differentiation. At D20, the cells were purified by FACS by P75NTR and cKIT cell surface marker, and double positive population were sorted out. The sorted melanoblasts were plated onto dried PO/LM/FN dishes as droplets. After 30 minutes, melanocyte medium was slowly added to the plate. The melanocyte medium contains neurobasal (NB) medium supplemented with 1-glutamine (Gibco, 25030-164), N2 (Stem Cell Technologies, 07156), B27 (Life Technologies, 17504044) and NEAA, SCF (50 ng/mL, R&D, 255-SC-MTO), cAMP (500 μM, Sigma, D0627), FGF2 (10 ng/mL, R&D, 233-FB/CF), CHIR (3 μM, R&D, 4423), BMP (25 ng/mL, R&D, 314-BP), EDN3 (100 nM, American Peptide company, 88-5-10B). The cells are fed every 2-3 days and passaged when the cells reach 70-80% of confluency, using Accutase for 20 min at 37° C. for cell detachment. The cells were fixed for immunostaining or collected for gene expression analysis at different days of differentiation.

Immunostaining of tissue sections. The gut tissue collected from mouse was cleaned with cold PBS using a syringe with a pipette tip stuck to the end. The gut tubes were then opened using scissors and flatted as a sheet. The flatted gut tubes were rolled up (“Swiss Roll” technique) and were fixed with 4% PFA overnight at 4° C. They were then washed 3 times with PBS and transferred to 70% ethanol for Paraffin embedding. The paraffin embedding, slide preparation and Hematoxylin and Eosin (H&E) staining were performed by the Sloan Kettering Institute, Molecular Cytology Core. For immunofluorescent staining, the slides were brought to room temperature for 1 hour before staining. We used the Trilogy kit (Cat #920P-07) from Cell Marque and followed the protocols from the manufacturer for antigen retrieval. In brief, the slides were placed in Trilogy buffer and subjected to high pressure and temperature using an Electric Pressure Cooker set to “high” for 15 minutes. The slides were rinsed in clean hot Trilogy buffer for 5 minutes and washed 3 times in PBS. The slides were then processed following the normal immunostaining protocol as described above for cultured cells before being sealed with anti-fade medium and cover glass prior to imaging.

Migration and co-differentiation assays. Transwell assay: to test capacity of NC cells for invasion, we used the CytoSelect 24-Well Cell Invasion Assay, Basement (Cell Biolabs). We plated 200K cells per chamber in neural spheroids medium and added 500 μL of neural spheroid medium containing 10% fetal bovine serum to the lower well of the invasion plate and incubated the plate for 48 h at 37° C. in 5% CO2 atmosphere. Cells that crossed through the invasion chamber were stained with the Cell Stain Solution provided with the kit (Cell Biolabs) and examined under the microscope. The stained cells were then lysed and measured by plate reader for quantification. Migration assay: vagal or sacral neural spheroids were generated as described in enteric neuron differentiation section. Those spheroids were plated down on a 2D PO/LM/FN coated plated for assessing surface migration or embedded in 31) Matrigel for assessing migration within a Matrigel pellet. Pictures were taken at sequential time points post plating to trace the migration process. Scratch assay: vagal or sacral NC cells were plated on PO/LM/FN coated 24 well plates at density of 100×10³ cells per cm². After 24 h, the culture lawn is scratched manually using a pipette tip. Live images are taken at different time points after the scratch was made to trace the migration.

Transplantation of NC in adult colon. The procedure for colon transplantation has been previously described in (Fattahi et al., Nature 531, 105-109, 2016). All mouse procedures were performed following NIH guidelines and were approved by the local Institutional Animal Care and Use Committee (IACOCCA), the Institutional Biosafety Committee (IBC) as well as the Embryonic Stem Cell Research Committee (ESCRO). 4-6 weeks old male NSG (NOD.Cg-Prkdcscid Il2rgtm1Wjl/SzJ) mice or 2-3 weeks old Ednrb KO/NSG homozygous mice were used for these studies. Animal numbers were based on availability of homozygous hosts and on sufficient statistical power to detect large effects between treatment versus control as well as for demonstrating robustness of migration behavior (NSG). Animals were randomly selected for the various treatment models but assuring for equal distribution of male/female ratio in each group. All in vivo experiments were performed in a blinded manner. Animals were anaesthetized with isoflurane (1%) throughout the procedure. A small abdominal incision was made, abdominal wall musculature lifted, and the caecum is exposed and exteriorized. Warm saline was used to keep the caecum moist. Then 20 μL of cell suspension (2-4 million GFP+ or RFP+ CD49D-purified human ES-cell-derived NC cells) in 70% Matrigel (BD Biosciences, 354234)/PBS or 20 μL of 70% Matrigel in PBS only (control-grafted animals) was slowly injected into the caecum (targeting the muscle layer) using a 27-gauge needle. Use of 70% matrigel as carrier for cell injection assured that the cells stayed in place after the injection and prevented backflow into the peritoneum. After injection that needle was withdrawn, and a Q-tip was placed over the injection site for 30 s to prevent bleeding. The caecum was returned to the abdominal cavity and the abdominal wall was closed using 4-0 vicryl and a taper needle in an interrupted suture pattern and the skin was closed using sterile wound clips. After wound closure animals were put on paper on top of their bedding and attended until conscious and preferably eating and drinking. The tissue was collected at different time points (ranging from two weeks to 9 months) after transplantation for histological analysis.

Multi-electrode array recording (MEA assay). hPSC-derived vagal NC or sacral NC were seeded onto poly-1-lysine-coated complementary metal oxide semiconductor multi-electrode arrays (CMOS-MEA) probes (3Brain). A 100-μL droplet of medium containing 150K cells was placed on the recording area. After 1 h incubation, 1.5 of ENS differentiation medium were added to the probe and replaced every 3-5 days. Recordings were performed at different time points. 1 minute of spontaneous activity was sampled from 4096 electrodes using the BioCAM system and analyzed using BrainWave 4 software. Spikes were detected using a sliding window algorithm on the raw channel traces applying a threshold for detection of 9 standard deviations. Bursts were defined as a minimum of 5 spikes occurring within a 100 ms window in a given channel.

Statistical analysis. Data are presented as Mean±SD and were derived from at least three independent experiments. Data on replicates (n) is given in figure legends. Statistical analysis was performed using the Unpaired t-test, also known as Student's t-test (comparing two groups) or ANOVA with Dunnett test (comparing multiple groups against control). Distribution of the raw data approximated normal distribution (t test) or Gaussian distribution (ANOVA test).

Although the present disclosure and certain of its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the present disclosure, processes, machines, manufacture, compositions of matter, or methods, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. An in vitro method for inducing differentiation of stem cells, comprising activation of wingless (Wnt) signaling, activation of fibroblast growth factor (FGF) signaling, and sacral neural crest patterning in the stem cells to obtain a population of differentiated cells expressing at least one marker indicating a sacral neural crest lineage; or comprising contacting the stem cells with at least one activator of Wnt signaling, at least one activator of FGF signaling, and at least one molecule that induces sacral neural crest patterning.

2. The method of claim 1, wherein the cells are contacted with the at least one molecule that induces sacral neural crest patterning for at least about 1 days, and/or the sacral neural crest patterning is induced for at least about 1 days.

3. The method of claim 1, wherein the cells are contacted with the at least one activator of FGF signaling for at least about 1 days, and/or the activation of FGF signaling is induced for at least about 1 days.

4. The method of claim 1, wherein the cells are contacted with the at least one activator of Wnt signaling for at least about 6 days, and/or the activation of Wnt signaling is induced for at least about 6 days.

5. The method of claim 1, wherein the at least one molecule that induces sacral neural crest patterning is selected from the group consisting of a member of transforming growth factor β (TGFβ) family, GDF11, GDF8, and combinations thereof.

6. The method of claim 1, wherein the at least one activator of FGF signaling is selected from the group consisting of FGF1, FGF2, FGF4, FGF6, FGF7, FGF8, FGF17, FGF18, and combinations thereof.

7. The method of claim 1, wherein the at least one activator of Wnt signaling is selected from the group consisting of an inhibitor of glycogen synthase kinase 3β (GSK3β) signaling, CHIR99021, CHIR98014, AMBMP hydrochloride, LP 922056, Lithium, BIO, SB-216763, Wnt3A, Wnt1, Wnt5a, derivatives thereof, and combinations thereof.

8. The method of claim 1, wherein the cells are further contacted with at least one inhibitor of Small Mothers Against Decapentaplegic (SMAD) signaling, and/or the method further comprises inducing inhibition of SMAD signaling.

9. The method of claim 8, wherein the at least one inhibitor of SMAD signaling comprises an inhibitor of TGFβ/Activin-Nodal signaling, and/or the inhibition of SMAD signaling comprises inhibition of TGFβ/Activin-Nodal signaling.

10. The method of claim 1, wherein the cells are contacted with at least one bone morphogenetic protein (BMP), and/or the method further comprises inducing activation of BMP signaling.

11. The method of claim 10, wherein the at least one BMP is selected from the group consisting of BMP1, BMP2, BMP3, BMP4, BMP5, BMP6, BMP7, BMP8a, BMP8b, BMP10, BMP11, BMP15, and combinations thereof.

12. The method of claim 1, wherein at least about 70% of the cells express the at least one sacral neural crest lineage marker at least about 20 days from the initial contact of the stem cells with the at least one activator of Wnt signaling, and/or from the initiation of the induction of activation of Wnt signaling.

13. The method of claim 1, wherein the at least one sacral neural crest lineage marker is selected from the group consisting of Hox10, Hox11, Hox12, and Hox13, and combinations thereof.

14. The method of claim 1, wherein the stem cells are selected from the group consisting of pluripotent stem cells, human stem cells, embryonic stem cells, induced pluripotent stem cells, parthenogenetic stem cells, primordial germ cell-like pluripotent stem cells, epiblast stem cells, and F-class pluripotent stem cells, enhanced pluripotent stem cells, naive stage pluripotent stem cells, and combinations thereof.

15. The method of claim 1, further comprising subjecting the differentiated cells to conditions favoring maturation of sacral neural crest lineage cells to cells that express at least one enteric neuron marker or at least one enteric glia cell marker.

16. The method of claim 15, wherein the conditions comprise contacting the differentiated cells with at least one growth factor, at least one Wnt activator, or a combination thereof.

17. The method of claim 15, wherein the at least one enteric neuron marker is selected from the group consisting of Tuj1, MAP2, PHOX2A, PHOX2B, TRKC, ASCL1, HAND2, EDNRB, 5HT, GABA, NOS, SST, TH, CH AT, DBH, Substance P, VIP, NPY, GnRH, CGRP, and combinations thereof; and/or wherein the at least one enteric glia cell marker is selected from the group consisting of GFAP, S100b, vimentin, conexin-43, SOX10, and combinations thereof.

18. A cell population of in vitro differentiated cells expressing at least one sacral neural crest lineage marker obtained by a method of claim 1.

19. A kit for inducing differentiation of stem cells, comprising:
(a) at least one activator of Wnt signaling;
(b) at least one activator of FGF signaling;
(c) at least one molecule that induces sacral neural crest patterning; and
(d) instructions for inducing differentiation of the stem cells into cells expressing at least one sacral neural crest lineage marker; or
comprising:
(a) at least one activator of Wnt signaling;
(b) at least one activator of FGF signaling;
(c) at least one molecule that induces sacral neural crest patterning;
(d) at least one growth factor,
(e) at least one Wnt activator; and
(f) instructions for inducing differentiation of the stem cells into cells expressing at least one enteric neuron marker.

20. A method of preventing and/or treating an enteric nervous system disorder in a subject in need thereof, comprising administering to the subject an effective amount of the cell population of claim 18.


SEQUENCE LISTING (6 sequences)

SEQ ID NO: 1 - DNA, 20 bp, synthetic construct
  misc_feature 1..20: note = Synthetic
  source 1..20: mol_type = other DNA
cctctcacacatgtgagggc

SEQ ID NO: 2 - DNA, 20 bp, synthetic construct
  misc_feature 1..20: note = Synthetic
  source 1..20: mol_type = other DNA
accttccatgtgaagcagca

SEQ ID NO: 3 - DNA, 20 bp, synthetic construct
  misc_feature 1..20: note = Synthetic
  source 1..20: mol_type = other DNA
tacctcttcctcccactcca

SEQ ID NO: 4 - DNA, 21 bp, synthetic construct
  misc_feature 1..21: note = Synthetic
  source 1..21: mol_type = other DNA
ccacagagatggttcgccagt

SEQ ID NO: 5 - DNA, 20 bp, synthetic construct
  misc_feature 1..20: note = Synthetic
  source 1..20: mol_type = other DNA
ctacacacccctcacccatc

SEQ ID NO: 6 - DNA, 25 bp, synthetic construct
  misc_feature 1..25: note = Synthetic
  source 1..25: mol_type = other DNA
ctaaaacctgctgtcctcaactatg